(19) United States
(12) Patent Application Publication
(10) Pub. No.: US 2025/0099491 A1
(43) Pub. Date: Mar. 27, 2025

(54) PREVENTION OR TREATMENT OF CARDIOVASCULAR DISEASES WITH HIGH PENETRATION PRODRUGS OF ASPIRIN AND OTHER NSAIDS

(71) Applicants: Techfields Inc., Peinceton, NJ (US); TECHFIELD PHARMA CO., LTD., Suzhou, Jiangsu (CN)

(72) Inventors: Chongxi YU, Princeton, NJ (US); Lina XU, Suzhou, Jiangsu (CN)

(73) Assignee: Techfields Inc., Princeton (JP)

(21) Appl. No.: 18/729,497

(22) PCT Filed: Jan. 12, 2023
(86) PCT No.: PCT/CN2023/071973

§ 371 Date: Jul. 16, 2024

(30) Foreign Application Priority Data
Jan. 17, 2022 (WO) PCT/CN2022/072270

(51) Int. Cl. A61K 31/606; A61K 9/00; A61K 31/616; A61K 31/625; A61P 9/10

(52) U.S. Cl. CPC A61K 31/606; A61K 9/0019; A61K 31/616; A61K 31/625; A61P 9/10

(57) ABSTRACT

The present disclosure provides 2-(diethylamino)ethyl acetoxybenzoate hydrochloride, other high penetration prodrugs of aspirin and other NSAIDs, and pharmaceutically acceptable salts thereof for use and methods thereof in the prevention or treatment of cardiovascular diseases and conditions. Pharmaceutical compositions, treatment kits and devices including 2-(diethylamino)ethyl acetoxybenzoate hydrochloride, other high penetration prodrugs of aspirin and other NSAIDs, and pharmaceutically acceptable salts thereof, as well as dosage forms, dosages, and methods of use thereof through topical administration are described.

### TECHNICAL FIELD OF THE DISCLOSURE

The present disclosure relates to the field of medical application, in particular the use of high penetration prodrugs of aspirin and other non-steroidal anti-inflammatory drugs (NSAIDs), and pharmaceutically acceptable salts thereof, for the prevention or treatment of cardiovascular diseases or conditions, especially by topical or transdermal administration.

### BACKGROUND OF THE DISCLOSURE

Aspirin has been used in humans and animal for more than one hundred years. Aspirin and other known NSAIDs with analgesic, antiphlogistic, antipyretic anti-inflammatory, or anti-platelet properties are used widely for treatment of symptoms in relation with various diseases, for example, cardiovascular diseases, inflammatory diseases and pains, such as rheumatic diseases, headaches, migraines, toothaches, back aches, muscle pain, post-operative pain, and the like.

Aspirin and other NSAIDs are usually administered through oral administration to reach the action site of a condition or disease. Unfortunately, the use of aspirin and other NSAIDs is accompanied by increased risks of significant gastrointestinal (GI) toxicity, including gastroduodenal bleeding, gastric ulcerations, gastritis, and GI perforation (see, e.g., Cohn S M, et al., J. Clin. Invest. 1997; 99 (6): 1367-1379; Tarnawski A S and Ahluwalia A., Curr. Med. Chem. 2012; 19 (1): 16-27; Fries J F, J. Rheumatol Suppl. 1991; 28:6-10; Garcia Rodriguez L A, et al., Arch. Intern Med. 1998; 158 (1): 33-9; and Richardson C, Emery P., Drug Saf. 1996; 15(4):249-60). Moreover, oral administration has limited efficacy in the prevention or treatment of certain diseases.

Therefore, there is still a need to develop methods that can fully realize the efficacy of aspirin and other NSAIDs, such as new alternative delivery routes, while avoiding GI toxicity.

### SUMMARY OF THE DISCLOSURE

The present disclosure meets the aforementioned need by providing high penetration prodrugs (HPPs) of aspirin and/or other NSAIDs, high penetration compositions (HPCs) thereof, use of the HPPs or HPCs, kits, therapeutic systems, dosage forms, devices using or comprising the HPPs or HPCs, and methods for treatment of various cardiovascular diseases and conditions.

In one aspect, the present disclosure provides a compound of Formula (I) as an HPP of aspirin or analogs thereof, which comprises a functional unit covalently linked to a transportational unit through a linker:

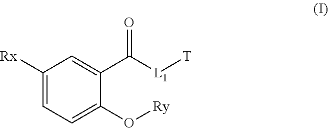

and stereoisomers and pharmaceutically acceptable salts thereof, wherein:
L₁ is a linker selected from O, S, NH, O—CH(L₂), O—(CH₂)ₙ, O—CH(L₂)—O—C(═O), O—CH(L₂)-O, S—CH(L₂)-O, and —O—C(═O)—, wherein n is an integer selected from 1 to 6;
L₂ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino;
T is a transportational unit comprising a protonatable amine group, for example, substituted or unsubstituted primary amine group, substituted or unsubstituted secondary amine group, substituted or unsubstituted tertiary amine group, or heterocyclyl group containing a protonatable nitrogen;
Rx is selected from hydrogen (H), 2,4-difluorophenyl, substituted and unsubstituted alkyl, substituted and unsubstituted cycloalkyl, substituted and unsubstituted heterocyclyl, substituted and unsubstituted alkoxy, substituted and unsubstituted alkenyl, substituted and unsubstituted alkynyl, substituted and unsubstituted aryl, and substituted and unsubstituted heteroaryl; and
Ry is selected from hydrogen (H), substituted and unsubstituted alkylcarbonyl, substituted and unsubstituted alkoxycarbonyl, substituted and unsubstituted benzoyl, substituted and unsubstituted alkyl, substituted and unsubstituted cycloalkyl, substituted and unsubstituted heterocyclyl, substituted and unsubstituted alkoxy, substituted and unsubstituted alkenyl, substituted and unsubstituted alkynyl, substituted and unsubstituted aryl, and substituted and unsubstituted heteroaryl.

The compound of Formula (I) can form a pharmaceutically acceptable salt with an acid through the protonatable transportational unit T, which salt is capable of penetrating one or more biological barriers.

In one aspect, the present disclosure provides a pharmaceutical composition comprising an HPP of aspirin or analog characterized by Formula (I), and a pharmaceutically acceptable carrier. Such a pharmaceutical composition constitutes a high penetration composition (HPC).

In one aspect, the present disclosure provides use of the HPPs or HPCs for prevention or treatment of various cardiovascular diseases or conditions through convenient topical administrations, which can overcome the GI toxicity altogether, and unexpectedly improve the efficacy of prevention or treatment, among numerous other advantages as compared with the traditional oral administration.

In one aspect, the present disclosure provides use of HPPs in the manufacture of a medicament for prevention or treatment of various cardiovascular diseases or conditions, such as strokes, angina, myocardial infarction, heart failure, coronary artery diseases, rheumatic heart disease, hypertensive heart disease, atrial fibrillation, congenital heart disease, endocarditis, aortic aneurysms, and peripheral artery disease.

In one aspect, the present disclosure provides a method of preventing or treating a disease or condition, the method comprising administering to a subject a therapeutically effective amount of an HPP or HPC disclosed herein. In some embodiments, the disease or condition is selected from strokes, angina, myocardial infarction, heart failure, coronary artery diseases, rheumatic heart disease, hypertensive heart disease, atrial fibrillation, congenital heart disease, endocarditis, aortic aneurysms, peripheral artery disease, and other cardiovascular diseases.

In one aspect, the present disclosure provides a kit including an HPP of aspirin or other NSAIDs, such as 2-(diethylamino)ethyl 2-acetoxybenzoate or 2-(diethylamino)ethyl acetoxybenzoate hydrochloride.

In one aspect, the present disclosure provides a therapeutic system including a composition comprising an HPP of aspirin or other NSAIDs, such as 2-(diethylamino)ethyl 2-acetoxybenzoate and/or a related high penetration prodrug of aspirin or 2-(diethylamino)ethyl acetoxybenzoate hydrochloride.

In one aspect, the present disclosure provides a dosage form comprising a certain concentration of HPP of aspirin or other NSAIDs, such as 2-(diethylamino)ethyl 2-acetoxybenzoate or 2-(diethylamino)ethyl acetoxybenzoate hydrochloride for the treatments of cardiovascular diseases.

In one aspect, the present disclosure provides a device capable of administering a certain unit dose of HPP of aspirin or other NSAIDs, such as 2-(diethylamino)ethyl 2-acetoxybenzoate or 2-(diethylamino)ethyl acetoxybenzoate hydrochloride to a subject having a cardiovascular disease or condition.

The HPPs or HPCs disclosed herein are capable of crossing one or more biological barriers and can be administered locally (e.g., topically or transdermally) to reach a location where a condition occurs but the existing drugs cannot reach significantly, thus avoiding drug-related gastrointestinal disorders or upper GI tract ulcer complications, such as bleeding episode, perforation, or gastric outlet obstruction, and unexpectedly improve the efficacy of prevention or treatment compared with oral administration. These and other advantages of the present disclosure will be better appreciated in view of the following detailed description, drawings, examples, and claims.

### BRIEF DESCRIPTION OF THE DRAWINGS

FIG. 1 illustrates the effect of 2-(diethylamino)ethyl acetoxybenzoate hydrochloride on brain infarct volume at Day 14 after Middle Cerebral Artery Occlusion (MCAO) (Mean±SEM, evaluated by TTC staining).

FIG. 2 illustrates the effect of 2-(diethylamino)ethyl acetoxybenzoate hydrochloride on the body weight change of rats after MCAO.

FIG. 3 illustrates the effect of 2-(diethylamino)ethyl acetoxybenzoate hydrochloride on neurological deficit scores of rats after MCAO by daily assessment in comparison with the vehicle group.

FIG. 4 illustrates the effect of 2-(diethylamino)ethyl hydroxybenzoate hydrochloride on brain infarct volume at Day 14 after MCAO (Mean±SEM, evaluated by TTC staining).

FIG. 5 illustrates the effect of 2-(diethylamino)ethyl hydroxybenzoate hydrochloride on the body weight change of rats after MCAO.

FIG. 6 illustrates the effect of 2-(diethylamino)ethyl hydroxybenzoate hydrochloride on neurological deficit scores of rats after MCAO by daily assessment in comparison with the vehicle group.

FIG. 7 illustrates the effect of (pyrrolidin-2-yl)methyl acetoxybenzoate hydrochloride on brain infarct volume at Day 14 after MCAO (Mean±SEM, evaluated by TTC staining).

FIG. 8 illustrates the effect of (pyrrolidin-2-yl)methyl acetoxybenzoate hydrochloride on the body weight change of rats after MCAO.

FIG. 9 illustrates the effect of (pyrrolidin-2-yl)methyl acetoxybenzoate hydrochloride on neurological deficit scores of rats after MCAO by daily assessment in comparison with the vehicle group.

FIG. 10 illustrates the effect of (pyrrolidin-2-yl)methyl 2′,4′-difluoro-4-acetoxy-[1,1′-biphenyl]-3-carboxylate hydrochloride on brain infarct volume at Day 14 after MCAO (Mean±SEM, evaluated by TTC staining).

FIG. 11 illustrates the effect of (pyrrolidin-2-yl)methyl 2′,4′-difluoro-4-acetoxy-[1,1′-biphenyl]-3-carboxylate hydrochloride on the body weight change of rats after MCAO.

FIG. 12 illustrates the effect of (pyrrolidin-2-yl)methyl 2′,4′-difluoro-4-acetoxy-[1,1′-biphenyl]-3-carboxylate hydrochloride on neurological deficit scores of rats after MCAO by daily assessment in comparison with the vehicle group.

FIG. 13 illustrates the effect of (pyrrolidin-2-yl)methyl 2-(2-acetoxybenzoyl)oxybenzoate hydrochloride on brain infarct volume at Day 14 day after MCAO (Mean±SEM, evaluated by TTC staining).

FIG. 14 illustrates the effect of (pyrrolidin-2-yl)methyl 2-(2-acetoxybenzoyl)oxybenzoate hydrochloride on body weight change of rats after MCAO.

FIG. 15 illustrates the effect of (pyrrolidin-2-yl)methyl 2-(2-acetoxybenzoyl)oxybenzoate hydrochloride on neurological deficit scores of rats after MCAO by daily assessment in comparison with the vehicle group.

FIG. 16 illustrates the effect of 2-(diethylamino)ethyl acetoxybenzoate hydrochloride on neurological deficit scores of monkeys in comparison with the vehicle and aspirin groups.

FIG. 17 illustrates the effect of 2-(diethylamino)ethyl acetoxybenzoate hydrochloride on the infarcted volume of monkeys in comparison with the vehicle and aspirin groups.

FIG. 18 illustrates the effect of 2-(diethylamino)ethyl acetoxybenzoate hydrochloride on the infarcted volume of minipigs in comparison with the vehicle and aspirin groups.

### DETAILED DESCRIPTION OF THE DISCLOSURE

In one aspect, the present disclosure provides HPPs of aspirin or analogs thereof, which comprise a functional unit covalently linked to a transportational unit through a linker, characterized by Formula (I):

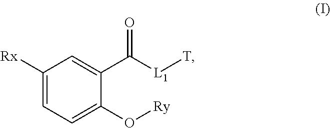

and stereoisomers and pharmaceutically acceptable salts thereof, wherein:
Rx is selected from hydrogen (H), 2,4-difluorophenyl, substituted and unsubstituted alkyl, substituted and unsubstituted cycloalkyl, substituted and unsubstituted heterocyclyl, substituted and unsubstituted alkoxy, substituted and unsubstituted alkenyl, substituted and unsubstituted alkynyl, substituted and unsubstituted aryl, and substituted and unsubstituted heteroaryl;
Ry is selected from hydrogen (H), substituted and unsubstituted alkylcarbonyl, substituted and unsubstituted alkoxycarbonyl, substituted and unsubstituted benzoyl, substituted and unsubstituted alkyl, substituted and unsubstituted cycloalkyl, substituted and unsubstituted heterocyclyl, substituted and unsubstituted alkoxy, substituted and unsubstituted alkenyl, substituted and unsubstituted alkynyl, substituted and unsubstituted aryl, and substituted and unsubstituted heteroaryl; preferably, Ry is 2-acetoxybenzoyl or 2-hydroxybenzoyl;
L₁ is a linker selected from O, S, NH, O—CH(L₂), O—(CH₂)ₙ, O—CH(L₂)—O—C(═O), O—CH(L₂)-O, S—CH(L₂)-O, and —O—C(═O)—, wherein n is an integer selected from 1 to 6;
L₂ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino;
T is a transportational unit comprising a protonatable amine group, for example, substituted or unsubstituted primary amine group, substituted or unsubstituted secondary amine group, substituted or unsubstituted tertiary amine group, or substituted or unsubstituted heterocyclyl containing a protonatable nitrogen; T can be selected from Structure W-1, Structure W-2, Structure W-3, Structure W-4, Structure W-5, and Structure W-6:

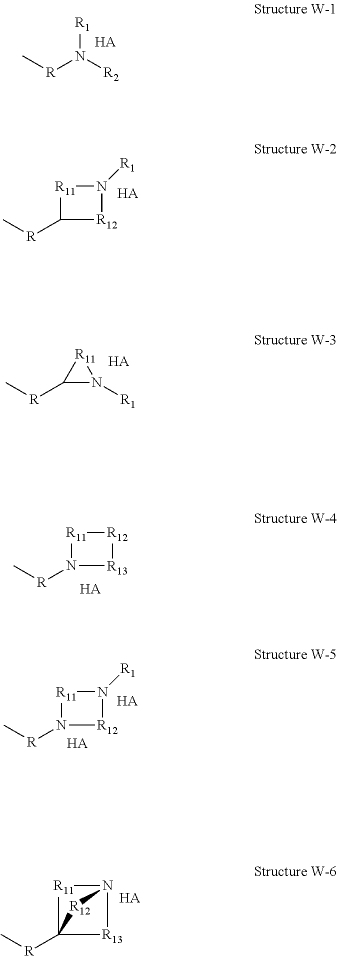

R at each occurrence is independently selected from a bond, substituted and unsubstituted alkylene, substituted and unsubstituted cycloalkylene, substituted and unsubstituted heterocyclylene, substituted and unsubstituted alkenylene, substituted and unsubstituted alkynylene, substituted and unsubstituted arylene, and substituted and unsubstituted heteroarylene, wherein any CH₂ in R may be optionally further replaced with O, S, or NR₃, wherein R₃ is hydrogen, C₁-C₆ alkyl, C₃-C₆ cycloalkyl, or C₆-C₁₀ aryl; preferably, R at each occurrence is-CH₂- or —CH₂—CH₂—;
R₁ and R₂ are independently selected from H, substituted and unsubstituted alkyl, substituted and unsubstituted cycloalkyl, substituted and unsubstituted heterocyclyl, substituted and unsubstituted alkyloxy, substituted and unsubstituted alkenyl, substituted and unsubstituted alkynyl, substituted and unsubstituted aryl, and substituted and unsubstituted heteroaryl; or alternatively R₁ and R₂ together with the nitrogen atom to which they are attached form an optionally substituted heterocyclyl, which optionally further comprises one or two additional heteroatom(s) independently selected from O, S, and N;
R₁₁, R₁₂, and R₁₃ are each independently a bond, an optionally substituted C₁-C₄ alkylene, or an optionally substituted C₂-C₄ alkenylene, wherein the alkylene and alkenylene optionally has one CH₂ group replaced by O, S, or NR₃; preferably, R₁₁, R₁₂, and R₁₃ are each independently-CH₂- or —CH₂—CH₂—;
wherein any of the R₁ in Strucure W-2, Structure W-3 or Structure W-5 and the adjacent R₁₁ together with the nitrogen atom to which they are attached may form an optionally substituted heterocyclic ring, which may optionally further comprise one or two additional heteroatom(s) independently selected from O, S, and N; and
wherein the R₁₁ and R₁₂ or R₁₁ and R₁₃ in Strucure W-2, Structure W-4, Structure W-5, or Structure W-6 may optionally be connected by an alkylene bridge, which is optionally substituted; and
wherein HA is selected from nothing and pharmaceutically acceptable acids, e.g., hydrochloride, hydrobromide, hydroiodide, nitric acid, sulfic acid, bisulfic acid, phosphoric acid, phosphorous acid, phosphonic acid, isonicotinic acid, acetic acid, lactic acid, salicylic acid, citric acid, tartaric acid, pantothenic acid, bitartaric acid, ascorbic acid, succinic acid, maleic acid, gentisinic acid, fumaric acid, gluconic acid, glucaronic acid, saccharic acid, formic acid, benzoic acid, glutamic acid, methanesulfonic acid, ethanesulfonic acid, benzenesulfonic acid, p-toluenesulfonic acid and pamoic acid;
provided, however, that the structure forms a stable compound with no covalent bonding principles being violated.

In some embodiments, when HA is nothing, the compound of Formula (I) is a free base.

In some embodiments, in the compound of Formula (I), Rx is hydrogen, halogen, hydroxyl, thiol, nitro, cyano, C₁-C₆ alkyl, C₁-C₆ alkoxy, C₁-C₆ alkylthio, or NR^{a}R^{b}, wherein R^{a} and R^{b} are same or different and are independently hydrogen or C₁-C₆ alkyl.

In some embodiments, in the compound of Formula (I), Ry is selected from hydrogen (H), CH₃CO—, CH₃CH₂CO—, 2-hydroxybenzoyl, and 2-acetoxybenzoyl, wherein the phenyl moiety of 2-hydroxybenzoyl or 2-acetoxybenzoyl is further optionally substituted by one to three substituents independently selected from halogen, hydroxyl, thiol, nitro, cyano, C₁-C₆ alkyl, C₁-C₆ alkoxy, C₁-C₆ alkylthio, phenyl, and NR^{a}R^{b}, wherein R^{a} and R^{b} are same or different and are independently hydrogen or C₁-C₆ alkyl, and wherein the phenyl is optionally substituted by one to five, sometimes preferably one to three, substituents independently selected from halogen, hydroxyl, thiol, nitro, cyano, C₁-C₆ alkyl, C₁-C₆ alkoxy, C₁-C₆ alkylthio, and NR^{a}R^{b}.

In some embodiments, in the compound of Formula (I), L₁ is O, S, NH, or O(CH₂)ₙ(n is 1, 2, 3 or 4).

In some embodiments, in the compound of Formula (I), R is a bond or C₁-C₆ alkylene.

In some embodiments, in the compound of Formula (I), T is Structure W-1, wherein R₁ and R₂ are each hydrogen or C₁-C₆ alkyl.

In some embodiments, in the compound of Formula (I), T is Structure W-2, Structure W-3, Structure W-4, Structure W-5, or Structure W-6, wherein R is a bond or C₁-C₄ alkylene; R₁ is hydrogen or C₁-C₆ alkyl; R₁₁ is a C₁-C₄ alkylene; R₁₂ and R₁₃ are independently a bond, CH₂, or CH₂CH₂.

In some embodiments, in the compound of Formula (I), T is a heterocyclyl selected from pyrrolidinyl, piperidinyl, piperazinyl, and morpholinyl.

In some embodiments, R₁₁, R₁₂, and R₁₃ are each independently selected from CH₂, CH₂CH₂, CH═CH, CH₂CH₂CH₂, CH═CHCH₂, CH₂CH₂CH₂CH₂, CH₂CH—CH—CH₂, CH₂CH₂CH₂CH₂CH₂, CH₂CH₂CH₂CH₂CH₂CH₂, each of which is optionally substituted.

The present disclosure encompasses any plausible combinations of the embodiments disclosed herein with regards to the structure of Formula (I). For example, in some embodiments, in the compound of Formula (I):

Rx is hydrogen, halogen, hydroxyl, thiol, nitro, cyano, C₁-C₆ alkyl, C₁-C₆ alkoxy, C₁-C₆ alkylthio, or NR^{a}R^{b}, wherein R^{a} and R^{b} are same or different and are independently hydrogen or C₁-C₆ alkyl;

Ry is selected from hydrogen (H), CH₃CO—, CH₃CH₂CO—, 2-hydroxybenzoyl, and 2-acetoxybenzoyl, wherein the phenyl moiety of 2-hydroxybenzoyl or 2-acetoxybenzoyl is further optionally substituted by one to three substituents independently selected from halogen, hydroxyl, thiol, nitro, cyano, C₁-C₆ alkyl, C₁-C₆ alkoxy, C₁-C₆ alkylthio, phenyl, and NR^{a}R^{b}, wherein R^{a} and R^{b} are same or different and are independently hydrogen or C₁-C₆ alkyl, and wherein the phenyl is optionally substituted by one to three substituents independently selected from halogen, hydroxyl, thiol, nitro, cyano, C₁-C₆ alkyl, C₁-C₆ alkoxy, C₁-C₆ alkylthio, and NR^{a}R^{b};

L₁ is O, S, NH, or O(CH₂)ₙ(n is 1 or 2); and

T is Structure W-1, wherein R is a bond or C₁-C₄ alkylene; and R₁ and R₂ are each hydrogen or C₁-C₆ alkyl; or

T is Structure W-2, Structure W-3, Structure W-4, Structure W-5, or Structure W-6, wherein R is a bond or C₁-C₄ alkylene; R₁ is hydrogen or C₁-C₄ alkyl; R₁₁ is a C₁-C₄ alkylene; R₁₂ and R₁₃ are independently a bond, CH₂, or CH₂CH₂.

In some embodiments, the disclosure provides an HPP compound selected from:

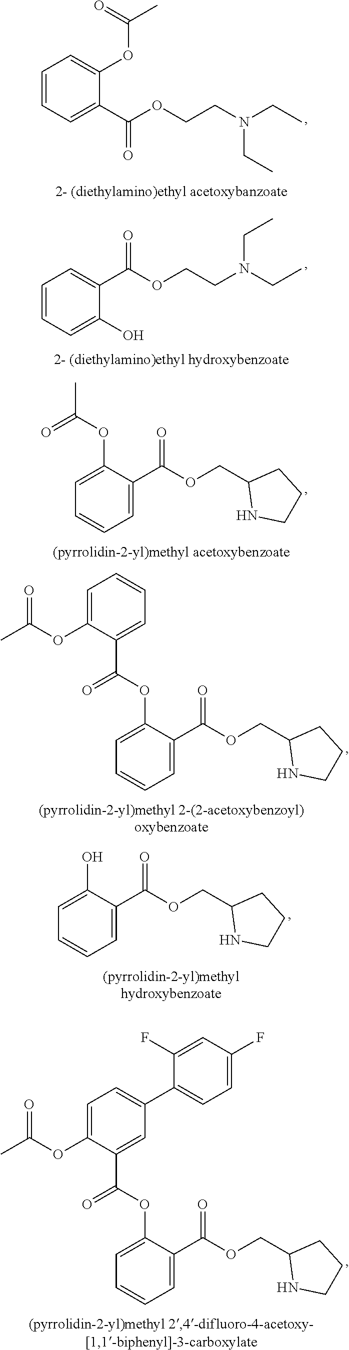

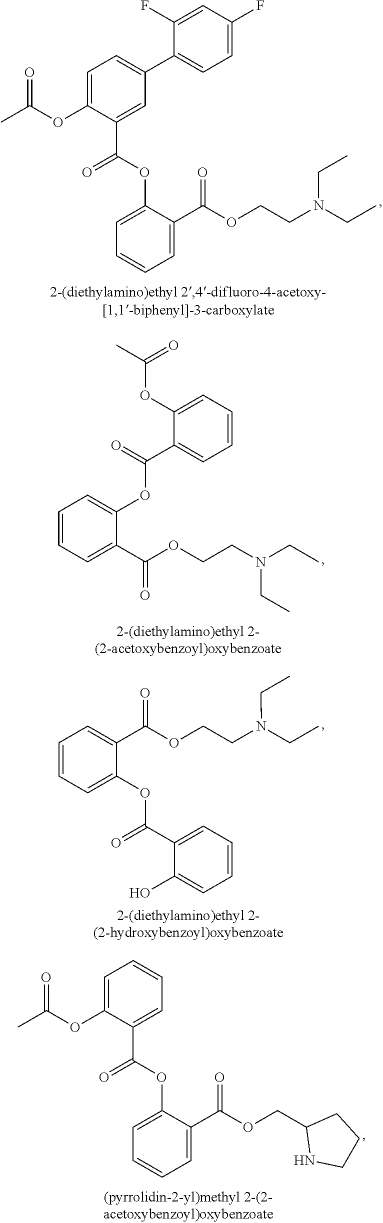

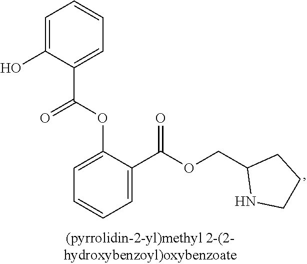 [SMILES: O=C(Oc1ccccc1C(=O)OCC1CCCN1)c1ccccc1O]

or a pharmaceutically acceptable salt thereof.

In some embodiments, the above pharmaceutically acceptable salt is formed with an acid selected from hydrochloride, hydrobromide, hydroiodide, nitric acid, sulfic acid, bisulfic acid, phosphoric acid, phosphorous acid, phosphonic acid, isonicotinic acid, acetic acid, lactic acid, salicylic acid, citric acid, tartaric acid, pantothenic acid, bitartaric acid, ascorbic acid, succinic acid, maleic acid, gentisinic acid, fumaric acid, gluconic acid, glucaronic acid, saccharic acid, formic acid, benzoic acid, glutamic acid, methanesulfonic acid, ethanesulfonic acid, benzenesulfonic acid, p-toluenesulfonic acid and pamoic acid. In a preferred embodiment, the acid is hydrochloride.

In some embodiments, the functional unit in the compound of Formula (I) (salicylic acid moiety of aspirin) can be replaced by a moiety of another NSAID, such as diflunil, acetyl diflunil, salsalate, and acetylsalsalate, or the like, with the hydroxyl on the carboxyl group of the NSAIDs being replaced.

In one aspect, the present disclosure provides use of the HPPs or HPCs for prevention or treatment of various cardiovascular diseases or conditions through convenient topical administrations, which can overcome the GI toxicity altogether, among numerous other advantages as compared with the traditional oral administration.

In some embodiments, the present disclosure provides use of 2-(diethylamino)ethyl acetoxybenzoate hydrochloride, (pyrrolidin-2-yl)methyl acetoxybenzoate hydrochloride, 2-(diethylamino)ethyl hydroxybenzoate hydrochloride, 2-(pyrrolidin-2-yl)methyl hydroxybenzoate hydrochloride, 2-(diethylamino)ethyl 2′,4′-difluoro-4-acetoxy-[1,1′-biphenyl]-3-carboxylate hydrochloride, (pyrrolidin-2-yl)methyl 2′,4′-difluoro-4-acetoxy-[1,1′-biphenyl]-3-carboxylate hydrochloride, 2-(diethylamino)ethyl 2′,4′-difluoro-4-hydroxyl-[1,1′-biphenyl]-3-carboxylate hydrochloride, (pyrrolidin-2-yl)methyl 2′,4′-difluoro-4-hydroxyl-[1,1′-biphenyl]-3-carboxylate hydrochloride, 2-(diethylamino)ethyl 2-(2-acetoxybenzoyl)oxybenzoate hydrochloride, (pyrrolidin-2-yl)methyl 2-(2-acetoxybenzoyl)oxybenzoate hydrochloride, 2-(diethylamino)ethyl 2-(2-hydroxybenzoyl)oxybenzoate hydrochloride, (pyrrolidin-2-yl)methyl 2-(2-hydroxybenzoyl)oxybenzoate hydroxybenzoyl)oxybenzoate hydrochloride, or other HPPs or HPCs of aspirin and other NSAIDs for prevention or treatment of strokes, angina, myocardial infarction, heart failure, coronary artery diseases, rheumatic heart disease, hypertensive heart disease, atrial fibrillation, congenital heart disease, endocarditis, aortic aneurysms, peripheral artery disease, and other cardiovascular diseases.

In one aspect, the present disclosure provides use of HPPs in the manufacture of a medicament for prevention or treatment of various cardiovascular diseases or conditions, such as strokes, angina, myocardial infarction, heart failure, coronary artery diseases, rheumatic heart disease, hypertensive heart disease, atrial fibrillation, congenital heart disease, endocarditis, aortic aneurysms, and peripheral artery disease.

In one aspect, the present disclosure relates to use of 2-(diethylamino)ethyl 2-acetoxybenzoate and/or related high penetration prodrugs of aspirin and/or other NSAIDs or pharmaceutically acceptable salts thereof in the manufacture of a medicament.

In some embodiments, the present disclosure provides use of 2-(diethylamino)ethyl acetoxybenzoate hydrochloride, (pyrrolidin-2-yl)methyl acetoxybenzoate hydrochloride, 2-(diethylamino)ethyl hydroxybenzoate hydrochloride, 2 (pyrrolidin-2-yl)methyl hydroxybenzoate hydrochloride, 2-(diethylamino)ethyl 2′,4′-difluoro-4-acetoxy-[1,1′-biphenyl]-3-carboxylate hydrochloride, (pyrrolidin-2-yl)methyl 2′,4′-difluoro-4-acetoxy-[1,1′-biphenyl]-3-carboxylate hydrochloride, 2-(diethylamino)ethyl 2′,4′-difluoro-4-hydroxy-[1,1′-biphenyl]-3-carboxylate hydrochloride, (pyrrolidin-2-yl)methyl 2′,4′-difluoro-4-hydroxy-[1,1′-biphenyl]-3-carboxylate hydrochloride, 2-(diethylamino)ethyl 2-(2-acetoxybenzoyl)oxybenzoate hydrochloride, (pyrrolidin-2-yl)methyl 2-(2-acetoxybenzoyl)oxybenzoate hydrochloride, 2-(diethylamino)ethyl 2-(2-hydroxybenzoyl)oxybenzoate hydrochloride, (pyrrolidin-2-yl)methyl 2-(2-hydroxybenzoyl)oxybenzoate hydrochloride, and other HPPs or HPCs of aspirin and other NSAIDs in the manufacture of a medicament for prevention or treatment of strokes, angina, myocardial infarction, heart failure, coronary artery diseases, rheumatic heart disease, hypertensive heart disease, atrial fibrillation, congenital heart disease, endocarditis, aortic aneurysms, peripheral artery disease, and other cardiovascular diseases.

In some embodiments, the present disclosure provides use of 2-(diethylamino)ethyl 2-acetoxybenzoate, 2-(diethylamino)ethyl acetoxybenzoate hydrochloride, or other HPPs of aspirin in the manufacture of a medicament for prevention or treatment of strokes, angina, myocardial infarction, heart failure, coronary artery diseases, rheumatic heart disease, hypertensive heart disease, atrial fibrillation, congenital heart disease, endocarditis, aortic aneurysms, peripheral artery disease, and other cardiovascular diseases.

In one aspect, the present disclosure provides a method of prevention or treatment of strokes, angina, myocardial infarction, heart failure, coronary artery diseases, rheumatic heart disease, hypertensive heart disease, atrial fibrillation, congenital heart disease, endocarditis, aortic aneurysms, peripheral artery disease, and other cardiovascular diseases through topical administration of HPPs or HPCs of aspirin and/or other NSAIDs.

In one aspect, the present disclosure provides a kit including an HPP of aspirin or other NSAIDs, such as 2-(diethylamino)ethyl 2-acetoxybenzoate or 2-(diethylamino)ethyl acetoxybenzoate hydrochloride.

In one aspect, the present disclosure provides a therapeutic system including a composition comprising an HPP of aspirin or other NSAIDs, such as 2-(diethylamino)ethyl 2-acetoxybenzoate and/or a related high penetration prodrug of aspirin or 2-(diethylamino)ethyl acetoxybenzoate hydrochloride.

In one aspect, the present disclosure provides a dosage form comprising a certain concentration of HPP of aspirin or other NSAIDs, such as 2-(diethylamino)ethyl 2-acetoxybenzoate or 2-(diethylamino)ethyl acetoxybenzoate hydrochloride for the treatments of cardiovascular diseases.

In one aspect, the present disclosure provides a device capable of administering a certain unit dose of HPP of aspirin or other NSAIDs, such as 2-(diethylamino)ethyl 2-acetoxybenzoate or 2-(diethylamino)ethyl acetoxybenzoate hydrochloride to a subject having a cardiovascular disease or condition.

In some embodiments, the present disclosure provides a method of prevention or treatment of a cardiovascular disease or condition in a subject, including topical administration of an HPP of aspirin or other NSAIDs, such as 2-(diethylamino)ethyl 2-acetoxybenzoate or 2-(diethylamino)ethyl acetoxybenzoate hydrochloride to the subject, in particular to one or more sites of the subject, in an amount of about 1 mg to about 1000 mg per time, in particular 3 mg to 200 mg per time.

In one aspect, the present disclosure provides an HPP of aspirin or other NSAIDs, such as 2-(diethylamino)ethyl 2-acetoxybenzoate, or 2-(diethylamino)ethyl acetoxybenzoate hydrochloride, for use in prevention or treatment of a cardiovascular disease or condition in a subject, wherein the HPP are topically administered to the subject, in particular to one or more sites of the subject, in an amount of about 1 mg to about 1000 mg per time, in particular 3 mg to 200 mg per time.

In some embodiments, the present disclosure provides use of an HPP of aspirin or other NSAIDs, such as 2-(diethylamino)ethyl 2-acetoxybenzoate or 2-(diethylamino)ethyl acetoxybenzoate hydrochloride in the manufacture of a medicament for topical administration for prevention or treatment of a cardiovascular disease or condition, wherein the HPP is topically administered to a subject, in particular to one or more sites of a subject, in an amount of about 1 mg to about 1000 mg per time, in particular 3 mg to 200 mg per time.

In some embodiments, the present disclosure provides a kit for prevention or treatment of a subject having a cardiovascular disease or condition, the kit comprising an HPP of aspirin or other NSAIDs, such as 2-(diethylamino)ethyl 2-acetoxybenzoate or 2-(diethylamino)ethyl acetoxybenzoate hydrochloride for topical administration to the subject, in particular to one or more sites of the subject, in an amount of about 1 mg to about 1000 mg per time, in particular 3 mg to 200 mg per time.

In some embodiments, the present disclosure provides a therapeutic system for prevention or treatment of a subject having a cardiovascular disease or condition, including a composition comprising an HPP of aspirin or other NSAIDs, such as 2-(diethylamino)ethyl 2-acetoxybenzoate or 2-(diethylamino)ethyl acetoxybenzoate hydrochloride, wherein the HPP presents as a free base or as a pharmaceutically acceptable salt, wherein in the system, the HPP is topically administered to the subject, in particular to one or more sites of the subject, in an amount of about 1 mg to about 1000 mg per time, in particular 3 mg to 200 mg per time.

In some embodiments, the present disclosure provides a method of prevention or treatment of a subject, including topical administrating an HPP of aspirin or other NSAIDs, such as 2-(diethylamino)ethyl 2-acetoxybenzoate or 2-(diethylamino)ethyl acetoxybenzoate hydrochloride to the subject, in particular to one or more sites of the subject, in an amount of about 5 μg/cm² to about 3 mg/cm² skin, in particular 5 μg/cm² to 3 mg/cm² skin, per time, for one time per day, two times per day, three times per day, or four times per day.

In some embodiments, the present disclosure provides an HPP of aspirin or other NSAIDs, such as 2-(diethylamino)ethyl 2-acetoxybenzoate, or 2-(diethylamino)ethyl acetoxybenzoate hydrochloride for use in prevention or treatment of a subject, wherein the HPP is topically administered to the subject, in particular to one or more sites of the subject, in an amount of about 5 μg/cm² to about 3 mg/cm² skin, in particular 5 μg/cm² to 3 mg/cm² skin, per time, for one time per day, two times per day, three times per day, or four times per day.

In some embodiments, the drug applied skin area is about 5 cm² to 15000 cm², in particular 25 cm² to 5000 cm², especially 100 cm² to 2500 cm².

In some embodiments, the present disclosure provides use of an HPP of aspirin or other NSAIDs, such as 2-(diethylamino)ethyl 2-acetoxybenzoate or 2-(diethylamino)ethyl acetoxybenzoate hydrochloride, in the manufacture of a medicament, wherein the HPP in the medicament is topically administered to a subject, in particular to one or more sites of a subject, in an amount of about 5 μg/cm² to about 3 mg/cm² skin, in particular 5 μg/cm² to 3 mg/cm² skin, per time.

In some embodiments, the present disclosure provides a kit for prevention or treatment of a subject, including an HPP of aspirin or other NSAIDs, such as 2-(diethylamino)ethyl 2-acetoxybenzoate or 2-(diethylamino)ethyl acetoxybenzoate hydrochloride, for being topically administered to the subject, in particular to one or more sites of the subject, in an amount of about 5 μg/cm² to about 3 mg/cm² skin, in particular 5 μg/cm² to 3 mg/cm² skin, per time.

In some embodiments, the present disclosure provides a therapeutic system for prevention or treatment of a subject, including a composition of which an HPP of aspirin or other NSAIDs, such as 2-(diethylamino)ethyl 2-acetoxybenzoate or 2-(diethylamino)ethyl acetoxybenzoate hydrochloride, is an active ingredient, the HPP is present as a free base or as a pharmaceutically acceptable salt, and wherein in the system, the HPP is topically administered to the subject, in particular to one or more sites of the subject, in an amount of about 5 μg/cm² to about 3 mg/cm² skin, in particular 5 μg/cm² to 3 mg/cm² skin, per time.

In some embodiments, the present disclosure provides a dosage form, wherein a concentration of an HPP of aspirin or other NSAIDs, such as 2-(diethylamino)ethyl 2-acetoxybenzoate or 2-(diethylamino)ethyl acetoxybenzoate hydrochloride, in the dosage form is about 3 mg/mL to about 200 mg/mL, in particular 10 mg/mL to 100 mg/mL, or about 3 mg/g to about 200 mg/g, in particular 10 mg/g to 100 mg/g.

In some embodiments, the present disclosure provides a device capable of administering a unit dose of about 0.1 mg to about 30 mg, in particular 0.1 mg to 30 mg, of an HPP of aspirin or other NSAIDs, such as 2-(diethylamino)ethyl 2-acetoxybenzoate or 2-(diethylamino)ethyl acetoxybenzoate hydrochloride, to a subject.

In some embodiments, the present disclosure provides a spray capable of spraying a unit dose of about 0.1 mg to about 30 mg, in particular 0.1 mg to 30 mg, of an HPP of aspirin or other NSAIDs, such as 2-(diethylamino)ethyl 2-acetoxybenzoate or 2-(diethylamino)ethyl acetoxybenzoate hydrochloride.

In one aspect, the present disclosure is intended to assess the efficacy and safety of an HPP of aspirin or other NSAIDs, such as 2-(diethylamino)ethyl 2-acetoxybenzoate or 2-(diethylamino)ethyl acetoxybenzoate hydrochloride, when administered to the skin as a topical spray in subjects with cardiovascular diseases.

In some embodiments, the present disclosure provides a pharmaceutical composition capable of penetrating biological barriers and methods of using the pharmaceutical composition for preventing or treating cardiovascular diseases, especially strokes, myocardial infarction, heart failure, angina, rheumatic heart disease, hypertensive heart disease, atrial fibrillation, congenital heart disease, endocarditis, aortic aneurysms, peripheral artery disease of humans and animals.

Unless defined otherwise, all technical and scientific terms used herein have the same meaning as commonly understood by one of ordinary skill in the art to which this invention belongs.

The term “cardiovascular diseases” refers to a group of disorders of the heart and blood vessels, including:
coronary heart disease—a disease of the blood vessels supplying the heart muscle;
cerebrovascular disease—a disease of the blood vessels supplying the brain;
peripheral arterial disease—a disease of blood vessels supplying the arms and legs;
rheumatic heart disease—damage to the heart muscle and heart valves from rheumatic fever, caused by streptococcal bacteria;
congenital heart disease—birth defects that affect the normal development and functioning of the heart caused by malformations of the heart structure from birth; and
deep vein thrombosis and pulmonary embolism—blood clots in the leg veins, which can dislodge and move to the heart and lungs.

The term “alkyl” refers to a branched or unbranched monovalent aliphatic hydrocarbon radical derived from an alkane by removal of one hydrogen atom. In certain embodiments, an alkyl group contains 1 to 8 carbons. In certain embodiments, sometimes preferably, an alkyl group contains 1 to 6 carbons, and in certain embodiments, sometimes more preferably, an alkyl group contains 1 to 4 carbons. Examples of alkyl include, but are not limited to, methyl, ethyl, propyl, isopropyl, butyl, isobutyl, t-butyl, pentyl, hexyl, heptyl, octyl, nonyl, decyl, undecyl, and dodecyl, or the like. In certain embodiments, an alkyl group contains 1 to 12 carbons. The alkyl group can be substituted or unsubstituted.

The term “alkenyl” refers to any univalent aliphatic hydrocarbon radical derived from an alkene by removal of one hydrogen atom. In certain embodiments, an alkenyl group contains 2 to 12 carbons. In certain embodiments, an alkenyl group contains 2 to 8 carbons. In certain embodiments, sometimes preferably, an alkenyl group contains 2 to 6 carbons, and in certain embodiments, sometimes more preferably, an alkenyl group contains 2 to 4 carbons. Examples of alkenyl include, but are not limited to, ethenyl, propenyl, butenyl, isobutenyl, pentenyl, hexenyl, heptenyl, octenyl, nonenyl, decenyl, undecenyl, dodecenyl, or the like. The alkenyl group can be substituted or unsubstituted.

The term “alkynyl” refers to a univalent aliphatic hydrocarbon radical derived from an alkyne by removal of one hydrogen atom. In certain embodiments, an alkynyl group contains 2 to 12 carbons. In certain embodiments, an alkynyl group contains 2 to 8 carbons. In certain embodiments, sometimes preferably, an alkynyl group contains 2 to 6 carbons, and in certain embodiments, sometimes more preferably, an alkynyl group contains 2 to 4 carbons. Examples of alkynyl include, but are not limited to, ethynyl, propynyl, butynyl, isobutynyl, pentynyl, hexynyl, heptynyl, octynyl, nonynyl, decynyl, undecynyl, dodecynyl, or the like. The alkynyl group can be substituted or unsubstituted.

The term “cycloalkyl” refers to any univalent radical formed by removal of one hydrogen atom from a cycloalkane. In certain embodiments, cycloalkyl group contains 3 to 10 carbons. In certain embodiments, cycloalkyl group contains 3 to 8 carbons. In certain embodiments, sometimes preferably, cycloalkyl group contains 3 to 6 carbons. Examples of cycloalkyl include, but are not limited to, cyclopropyl, cyclobutyl, cyclopentyl, cyclohexyl, cycloheptyl, cyclooctyl, cyclononyl, cyclodecyl, cycloundecyl and cyclododecyl. The cycloalkyl is optionally substituted or unsubstituted.

The term “heterocyclyl” refers to a cycloalkyl wherein at least one ring atom is a non-carbon atom. Examples of the non-carbon ring atom include, but are not limited to, S, O and N. Representative examples of monocyclic heterocyclyls include, but are not limited to, pyrrolidyl, piperidyl, piperazinyl, morpholinyl, sulfo-morpholinyl, homopiperazinyl, and so on.

The term “alkylene” refers to a saturated linear or branched divalent aliphatic hydrocarbon group, derived by removing two hydrogen atoms the parent alkane. The straight or branched chain group containing 1 to 12 carbon atom(s) (such as 1, 2, 3, 4, 5, 6, 7, 8, 9, 10, 11 and 12 carbon atom(s)), preferably has 1 to 8 carbon atom(s), more preferably 1 to 6 carbon atom(s), and sometimes more preferably 1 to 4 carbon atom(s). Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—), etc. The alkylene group can be substituted or unsubstituted.

The term “alkenylene” refers to an alkylene defined as above that has at least two carbon atoms and at least one carbon-carbon double bond, preferably C₂₋₁₂ alkenylene, more preferably C₂₋₈ alkenylene, sometimes more preferably C₂₋₆ alkenylene, and sometimes even more preferably C₂₋₄ alkenylene. Non-limiting examples of alkenylene groups include, but are not limited to, —CH═CH—, —CH═CHCH₂—, —CH═CHCH₂CH₂—, —CH₂CH═CHCH₂-etc. The alkenylene group can be substituted or unsubstituted.

The term “aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl group can be substituted or unsubstituted.

The term “heteroaryl” refers to an 5 to 14 membered aryl system having 1 to 4 heteroatom(s) selected from O, S and N as ring atoms. Preferably a heteroaryl is 5- to 10-membered (such as 5, 6, 7, 8, 9 and 10 membered), more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl can be fused with the ring of an aryl, heterocyclyl or cycloalkyl, wherein the ring bound to parent structure is heteroaryl. The heteroaryl group can be substituted or unsubstituted.

The term “alkoxy” refers to both an —O-(alkyl), for example, methoxy, ethoxy, propoxy, butoxy, and the like.

The term “cycloalkoxy” refers to —O-(cycloalkyl), for example, cyclopropyloxy, cyclobutyloxy, cyclopentyloxy, cyclohexyloxy, and the like.

The term “bond” refers to a covalent bond using a sign of “—”.

The term “hydroxyl” refers to an —OH group.

The term “halogen” refers to fluoro, chloro, bromo or iodo atoms.

The term “amino” refers to a —NH₂ group.

The term “alkylthio” refers to alkyl-S—.

The term “alkylamino” refers to “alkyl-NH—”, or sometimes dialkyl amino (—NR^{a}R^{b}), where the two alkyl groups (R^{a} and R^{b}) can be the same or different. Sometimes preferably, the alkyl group is a C₁-C₆ alkyl, and sometimes more preferably, the alkyl is a C₁-C₄ alkyl. Examples of alkylamino include, but are not limited to, CH₃—NH—, —N(CH₃)₂, —N(CH₂CH₃)₂, —NHCH₂CH₃, —N(CH₃) (CH₂CH₃), —NH-Bu^{t}, —N(CH₃) (Bu^{t}), or the like.

The term “cyano” refers to a —CN group.

The term “haloalkyl” means an alkyl group substituted by one or more halogen atoms, wherein the halogen atoms can be the same or different.

The term “nitro” refers to a —NO₂ group.

The term “oxo group” refers to a ═O group.

The term “carboxyl” refers to a —C(O)OH group.

The term “alkoxycarbonyl” refers to a —C(O)O(alkyl) group.

The term “alkylcarbonyl” refers to a —C(O)-alkyl group.

The term “optional” or “optionally” means that the event or circumstance described subsequently can, but need not, occur, and the description includes the instances in which the event or circumstance may or may not occur. For example, “the heterocyclyl group optionally substituted by an alkyl” means that an alkyl group can be, but need not be, present, and the description includes the case of the heterocyclyl group being substituted with an alkyl and the heterocyclyl group being not substituted with an alkyl.

The term “substituted” refers to one or more hydrogen atoms in the group, preferably up to 5, more preferably 1 to 3 hydrogen atom(s), independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

The term “covalent bonding principle”, as used herein, refers to those basic rules and principles in formation of covalent bonds in an organic compound, as generally understood by a person of ordinary skill in the art. For example, a carbon atom is tetravalent and can form only four covalent bonds (e.g., four single bonds, or a double bonds plus two single bonds, etc.), an oxygen is divalent and can only form two covalent bonds (two single bond in-O—, or a double bond in ═O).

The term “prodrug” refers to a compound that can be transformed in vivo to yield the active parent compound under physiological conditions, such as through hydrolysis in blood. Common examples include, but are not limited to, ester and amide forms of a compound having an active form bearing a carboxylic acid moiety. In particular, the present disclosure provides a unique class of prodrugs, namely “high penetration prodrugs”, as defined in the disclosure.

When any group in any HPP structure is indicated to be either “substituted” and/or “unsubstituted”, it means that the group can be optionally substituted by one or more, preferably one to five, and sometimes more preferably one to three, substituents independently selected from halogen, cyano, nitro, amino, alkyl, haloalkyl, alkoxy, haloalkoxy, aryl, alkylthio, alkylamino, alkylsulfonyl (alkylsulfone), alkylsulfoxyl (alkylsulfoxide), acyloxy, carboxylic acid, carboxylic ester, and carboxamide groups, or the like. The alkyl groups can be 1-10 carbon atoms, sometimes preferably 1-6 carbon atoms, sometimes more preferably 1-4 carbon atoms. The esters can be the esters of C₁ to C₁₀ alcohols, sometimes preferably C₁ to C₆ alcohols, sometimes more preferably C₁ to C₄ alcohols.

In some embodiments, when an alkyl, alkenyl, alkynyl, cycloalkyl, heterocyclyl, aryl, heteroaryl group, or the like, or a moiety thereof, is substituted, the substituent group(s) can be substituted at any available connection point(s), and the substituents can be one or more, sometimes preferably 1 to 5, and sometimes more preferably 1 to 3, group(s) independently selected from C₁-C₆ alkyl, halogen, C₁-C₆ alkoxy, C₁-C₆ alkenyl, C₁-C₆ alkynyl, C₁-C₆ alkylthio, C₁-C₆ alkylamino, di-(C₁-C₆ alkyl)amino, thiol, hydroxyl, nitro, cyano, amino, C₃-C₆ cycloalkyl, 5- to 10-membered heterocyclyl, C₆-C₁₀ aryl, 5- to 10-membered heteroaryl, C₃-C₆ cycloalkoxy, C₁-C₆ cycloalkylthio, 5- to 10-membered heterocyclylthio and oxo group. In some embodiments, sometimes preferably, the substituents are independently selected from C₁-C₆ alkyl, halogen, C₁-C₆ alkoxy, C₁-C₆ alkylthio, C₁-C₆ alkylamino, di-(C₁-C₆ alkyl)amino, thiol, hydroxyl, nitro, cyano, amino, and oxo group. In some embodiments, sometimes more preferably, the substituents are independently selected from C₁-C₄ alkyl, halogen, C₁-C₄ alkoxy, C₁-C₄ alkylthio, C₁-C₄ alkylamino, di-(C₁-C₄ alkyl)amino, thiol, hydroxyl, nitro, cyano, and amino. As a person of ordinary skill in the art would understand, an oxo (═O) group cannot be a substituent of an aryl or heteroaryl group, or at an unsaturated carbon in any other group.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “a” and “an” and “the” and similar references in the context of describing the invention (especially in the context of the following claims) are to be construed to cover both the singular and the plural, unless otherwise indicated herein or clearly contradicted by context. The use of the word “a” or “an” when used in conjunction with the term “comprising” in the claims and/or the specification may mean “one”, but it is also consistent with the meaning of “one or more”, “at least one”, and “one or more than one”.

Where the plural form is used for compounds, salts, and the like, this is taken to mean also a single compound, salt, or the like.

The term “and/or” as used herein refers to and encompasses any and all possible combinations of one or more of the associated listed items. When used in a list of two or more items, the term “and/or” means that any one of the listed items can be employed by itself, or any combination of two or more of the listed items can be employed. For example, if a composition, a combination, a constitution, a juxtaposition, or a group is described as including (or comprising) components A, B, C, and/or D, the composition can contain A alone; B alone; C alone; D alone; A and B in combination; A and C in combination; A and D in combination; B and C in combination; B and D in combination; C and D in combination; A, B, and C in combination; A, B, and D in combination; A, C, and D in combination; B, C, and D in combination; or A, B, C, and D in combination.

Throughout this application, the term “about” or “approximately” is used to indicate that a value includes the inherent variation of error for the device, the method being employed to determine the value, or the variation that exists among the study subjects. In one aspect, the terms “about” or “approximately” usually mean within 10%, in particular within 9%, in particular within 8%, in particular within 7%, in particular within 6%, in particular within 5%, in particular within 4%, in particular within 3%, in particular within 2%, in particular within 1%, in particular within 0.5% of a given value or range.

The term “treat”, “treating” or “treatment” as used herein comprises treatment or therapeutic regimen relieving, reducing or alleviating at least one symptom in a subject or effecting a delay of progression of a proliferative disorder. For example, treatment can be the diminishment of one or several symptoms of a disorder or complete eradication of a disorder, such as stroke, myocardial infarction, and/or cardiovascular diseases. Within the meaning of the present disclosure, the term “treat” also denotes to arrest, delay the onset (i.e., the period prior to clinical manifestation of a disorder) and/or reduce the risk of developing or worsening a disorder.

In some embodiments, the term “dose”, as used herein, means the amount of a drug or active component taken each time by an individual subject, in particular the total amount of a drug or active component taken each time by an individual subject, for one site.

In some embodiments, the term “dosage form”, as used herein, means a unit of administration of an active agent. Examples of dosage forms include tablets, capsules, injections, suspensions, liquids, emulsions, creams, ointments, suppositories, inhalable forms, transdermal forms, and the like.

In some embodiments, the term “unit dose” or “dosage unit” refers to a dosage form that is configured to deliver a specified quantity or dosage of composition or component thereof. Examples of dosage forms for topical administration include, but are not limited to, transdermal patch, cream, foam, gel, lotion, ointment, paste, powder, shake lotion, solid, sponge, tape, tincture, vapor, injection, drops, rinces, spray, and solution. A “unit dose” or “dosage unit” may be configured to provide a full unit dose or fraction thereof (e.g., ½, ⅓, or ¼ of a dose). A predetermined quantity in each unit dose can depend on factors that include, but are not limited to, the unique characteristics of the active compound and the particular therapeutic effect to be achieved, and the limitations inherent in the art of creating and administering such unit doses. For instance, a unit dose may be, a transdermal patch, a spray, i.e., once spray in the spray application, a droplet of the dropping application, a certain length of the tape, rice-sized or bean-sized ointment, or a scoop or a spoon of ointment. Unit dose measuring devices, such as a cup, scoop, syringe, dropper, spoon, or colonic irrigation device, may hold the dosage form, for instance cream, foam, gel, lotion, ointment, paste, powder, shake lotion and solid, a measured quantity of composition equaling a full unit dose or fraction thereof (e.g., ½, ⅓, or ¼ of a dose). There may be a single unit dose, or multiple unit doses, in a single dose of administration. The kit may include instructions regarding the size of the unit dose, or fraction thereof.

The term “pharmaceutically acceptable” is defined herein to refer to those compounds, materials, compositions and/or dosage forms, which are, within the scope of sound medical judgment, suitable for contact with the tissues of a subject without excessive toxicity, irritation allergic response and other problem complications commensurate with a reasonable benefit/risk ratio.

The term “pharmaceutical composition” is defined herein to refer to a substance or a mixture or solution containing at least one therapeutic agent to be administered to a subject, in order to prevent or treat, in particular to treat a particular disease or condition affecting the subject.

The term “other NSAIDs” as used herein refers to any NSAIDs other than aspirin, in particular salicylic acid, diflunil, acetyl diflunil, salsalate, and acetylsalsalate.

The term “pharmaceutically acceptable salt” refers to those salts of compounds of the invention that are safe for administration to a subject. For a review on pharmaceutically acceptable salts see Berge, et al., J. Pharm. Sci., 1977, 66, 1-19, which is incorporated herein by reference.

It is understood that the therapeutic agent may be administered each day in a single unit dose or multiple unit doses and/or administered each day in a single dose (once per day, q.d.) or divided doses (more than once per day, e.g., twice per day, b.i.d.).

The term “day” as used herein refers to either one calendar day in any time zone or one 24-hour period.

The terms “patient” or “subject” is intended to include animals, including warm-blooded animals. Examples of patients include mammals, e.g., humans, dogs, cows, horses, pigs, sheep, goats, cats, mice, rabbits, rats, and transgenic non-human animals. In some embodiments, the patient is a human, e.g., a human suffering from, at risk of suffering from, or potentially capable of suffering from a disease, for instance suffering from stroke, myocardial infarction, and/or cardiovascular diseases.

In some embodiments, the term “transdermal administration” means administration of a transdermal dose, unit dose or dosage form; the term “transdermally administering” means administering a transdermal dose, unit dose or dosage form; and the term “transdermally administered” means administered by a transdermal dose, unit dose or dosage form. That a patient and/or subject is “transdermally administered” is equivalent to that a patient and/or subject is subjected to a “transdermal administration”. “Transdermally administering” to a patient and/or subject is equivalent to subjecting a “transdermal administration” to a patient and/or subject.

In some embodiments, the term “site” (of the subject) is nearby skin and/or body surface of a human organ or body where a disease was found, for example, heart, brain, lung, head, neck, chest, arm, leg, and/or back, etc., itself having a disease, in particular poor blood flow, blood clotting, tissue inflammation, and/or cell death, etc., particularly the blood clotting, blood vessel inflammation, and/or tissue inflammation of brain, heart, lung, other organs and/or other tissues, etc., more particularly stroke, myocardial infarction, and/or other cardiovascular diseases.

In some embodiments, correspondingly, the term “administrating to the site” (of the subject) means administrating to: (a) the place on the nearby skin and/or body surface which is in correspondence with, or, close to the “site”; and/or (b) the place on the nearby skin and/or body surface that provides an accessible route to the “site”.

For instance, the site could be the nearby skin suffering from, at risk of suffering from, or potentially capable of suffering from the symptom of disease of brain, heart, lung and/or other organs, etc., more particularly stroke, myocardial infarction, and/or other cardiovascular diseases, while administrating to the site could be administrating to the skin and/or body surface near, in particular within about 1 cm to about 20 cm, in particular about 5 cm to about 15 cm, in particular a distance selected from about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm from the brain, heart, lung and/or other organs etc., and/or an environment about 1 cm to about 100 cm, in particular about 5 cm to about 50 cm, in particular a distance selected from about 5 cm, about 10 cm, about 15 cm, about 20 cm, about 25 cm, about 30 cm, about 35 cm, about 40 cm, about 45 cm, about 50, around all directions of the brain, heart, lung and/or other organ, etc.

In some embodiments, the term “close” or “close to” means within about 1 cm to about 100 cm, in particular about 10 cm to about 50 cm, in particular a distance selected from about 10 cm, about 15 cm, about 20 cm, about 30 cm, about 35 cm, about 40 cm, about 45 cm, about 50 cm, from the center of the site, i.e. brain, heart, lung, and/or other organs, etc.

In some embodiments, the term “symptom” refers to any symptoms such as diseases, inflammation, chest pain, palpitations, malaise, fever, shortness of breath, excessive tiredness, angina, painful leg and/or arm, edema, fatigue, syncope, headaches, weakness in the arm and/or leg, weakness of the muscles of the face, problems speaking, loss of vision, coordination problems, dizziness, loss of consciousness, et. al. In particular, the symptoms could include chest pain, shortness of breath, headache, dizziness, weakness in arm and/or leg, loss of consciousness related to blood clotting, in particular stroke, myocardial infarction, and/or other cardiovascular diseases.

In some embodiments of the previously mentioned embodiments, an HPP of aspirin or other NSAIDs, such as 2-(diethylamino)ethyl 2-acetoxybenzoate, or 2-(diethylamino)ethyl acetoxybenzoate hydrochloride is administered in an amount of about 1 mg to about 1000 mg, in particular about 3 mg to about 210 mg, especially about 35 mg to about 140 mg, per time, for once, twice, three times or four times per day, in particular, sometimes preferably, two times per day.

For example, it can be administered in an amount of 3.5 mg, 7 mg, 10.5 mg, 14 mg, 17.5 mg, 21 mg, 24.5 mg, 28 mg, 31.5 mg, 35 mg, 38.5 mg, 42 mg. 45.5 mg, 49 mg, 52.5 mg, 56 mg, 59.5 mg, 63 mg, 66.5 mg, 70 mg, 73.5 mg, 77 mg, 80.5 mg, 84 mg, 87.5 mg, 91 mg, 94.5 mg, 98 mg, 101.5 mg, 105 mg, 108.5 mg, 112 mg, 115.5 mg, 119 mg, 122.5 mg, 126 mg, 129.5 mg, 133 mg, 136.5 mg, 140 mg, 143.5 mg, 147 mg, 150.5 mg, 154 mg, 157.5 mg, 161 mg, 164.5 mg, 168 mg, 171.5 mg, 175 mg, 178.5 mg, 182 mg, 185.5 mg, 189 mg, 192.5 mg, 196 mg, 199.5 mg, 203 mg, 206.5 mg, and 210 mg per time, for once or twice per day. In some embodiments of the previously mentioned embodiments, an HPP of aspirin or other NSAIDs, such as 2-(diethylamino)ethyl 2-acetoxybenzoate, or 2-(diethylamino)ethyl acetoxybenzoate hydrochloride is administered in an amount of about 5 μg/cm² to about 15 mg/cm² skin, in particular about 10 μg/cm² to about 7 mg/cm² skin, about 30 μg/cm² to about 2 mg/cm² skin, about 50 μg/cm² to about 1.5 mg/cm² skin, about 70 μg/cm² to about 1 mg/cm² skin, sometimes preferably about 100 μg/cm² to about 700 μg/cm² skin, sometimes more preferably about 150 μg/cm² to about 500 μg/cm² skin, per time.

For example, it can be administered in an amount of 17.5 μg/cm², 35 μg/cm², 70 μg/cm², 140 μg/cm², 280 μg/cm², 560 μg/cm², 700 μg/cm², 1 mg/cm², 2 mg/cm², 3.5 mg/cm², or 7 mg/cm² skin, per dose.

In some embodiments of the previously mentioned embodiments, the subject is a warm-blooded animal. In some embodiments of the previously mentioned embodiments, the subject is a mammal. In some embodiments of the previously mentioned embodiments, the subject is a primate. In some embodiments of the previously mentioned embodiments, the subject is a human. In some embodiments of the previously mentioned embodiments, the subject is a minor. In some embodiments of the previously mentioned embodiments, the subject is a minor, the age of the minor is less than 16. In some embodiments of the previously mentioned embodiments, the subject is a human adult. In some embodiments of the previously mentioned embodiments, the age of the adult is more than or equal to 16.

In some embodiments of the previously mentioned embodiments, the subject is, and/or the medicament is for, a subject suffering from, at risk of suffering from, or potentially capable of suffering from symptoms. In some embodiments of the previously mentioned embodiments, the subject is, and/or the medicament is for, a subject suffering from, at risk of suffering from, or potentially capable of suffering from symptoms, in particular a subject suffering from, at risk of suffering from, or potentially capable of suffering from cardiovascular diseases or conditions, such as strokes, angina, myocardial infarction, heart failure, rheumatic heart disease, hypertensive heart disease, atrial fibrillation, congenital heart disease, endocarditis, aortic aneurysms, peripheral artery disease, and the like.

In some embodiments of the previously mentioned embodiments, the subject is, and/or the medicament is for, a subject suffering from, at risk of suffering from, or potentially capable of suffering from blood clotting (coagulation).

In some embodiments of the previously mentioned embodiments, the subject is, and/or the medicament is for, subject suffering from, at risk of suffering from, or potentially capable of suffering from diseases, inflammation, chest pain, palpitations, malaise, fever, shortness of breath, excessive tiredness, angina, painful leg and/or arm, edema, fatigue, syncope, headaches, weakness in the arm and/or leg, weakness of the muscles of the face, problems speaking, loss of vision, coordination problems, dizziness, loss of consciousness.

In some embodiments of the previously mentioned embodiments, a site of the subject includes one or more surfaces. In some embodiments of the previously mentioned embodiments, a site of the subject includes the neck surface, the chest surface, the back surface, the low back surface, the head surface, the chick surface, the shoulder surface, the arm surface, the hand surface, the leg surface, and/or the abdomen surface thereof.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is topically administered to one or more surfaces of the neck surface, the chest surface, the back surface, the low back surface, the head surface, the chick surface, the shoulder surface, the arm surface, the hand surface, the leg surface, and/or the abdomen surface thereof.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is topically administered by a transdermal administration. In some embodiments of the previously mentioned embodiments, the HPP is topically administered by a dosage form selected from one or more of transdermal patch, cream, foam, gel, lotion, ointment, paste, powder, shake lotion, solid, sponge, tape, tincture, vapor, injection, drops, rinces, spray, and solution. In some embodiments of the previously mentioned embodiments, the HPP is topically administered by a dosage form selected from one or more of transdermal drops, rinces and spray. In some embodiments of the previously mentioned embodiments, the HPP is topically administered by a spray. In some embodiments of the previously mentioned embodiments, the HPP is topically administered by a spray for subjects suffering from stroke, myocardial infarction, and/or cardiovascular diseases. In some embodiments of the previously mentioned embodiments, the HPP is topically administered by a drop. In some embodiments of the previously mentioned embodiments, the HPP is topically administered by a drop for subjects suffering from stroke, myocardial infarction, and/or cardiovascular diseases.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is topically administered by a dosage form including one or more unit doses. In some embodiments of the previously mentioned embodiments, the dosage from is selected from one or more of transdermal patch, cream, foam, gel, lotion, ointment, paste, powder, shake lotion, solid, sponge, tape, tincture, vapor, injection, drops, rinces, spray, and solution, and the dosage form including one or more unit doses.

In some embodiments of the previously mentioned embodiments, the dosage form is spray application. In some embodiments of the previously mentioned embodiments, the dosage form is a spray for subjects suffering from stroke, myocardial infarction, and/or cardiovascular diseases. In some embodiments of the previously mentioned embodiments, the dosage form is a plurality times of sprays, and the each one of the unit doses is once spray in the plurality of sprays. In some embodiments of the previously mentioned embodiments, the dosage form is a plurality of patches, and the each one of the unit doses is a patch in the plurality of patches. In some embodiments of the previously mentioned embodiments, the dosage form is drop application. In some embodiments of the previously mentioned embodiments, the dosage form is a drop for subjects suffering from stroke, myocardial infarction, and/or cardiovascular diseases. In some embodiments of the previously mentioned embodiments, the dosage form is a plurality times of drops, and the each one of the unit doses is once drop in the plurality of drops.

In some embodiments of the previously mentioned embodiments, a composition comprising an HPP of aspirin or other NSAIDs, such as 2-(diethylamino)ethyl 2-acetoxybenzoate or 2-(diethylamino)ethyl acetoxybenzoate hydrochloride, is topically administered to the subject. In some embodiments of the previously mentioned embodiments, the unit dose comprising a composition comprising 2-(diethylamino)ethyl acetoxybenzoate hydrochloride is topically administered to the subject.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is topically administered as dissolving in a solution. In some embodiments of the previously mentioned embodiments, the composition is a solution. In some embodiments of the previously mentioned embodiments, the composition is an alcohol solution. In some embodiments of the previously mentioned embodiments, the composition is an acetone solution. In some embodiments of the previously mentioned embodiments, the composition is a dimethyl sulfoxide solution. In some embodiments of the previously mentioned embodiments, the composition is an alcohol water solution. In some embodiments of the previously mentioned embodiments, the composition is an acetone water solution. In some embodiments of the previously mentioned embodiments, the composition is a dimethyl sulfoxide water solution. In some embodiments of the previously mentioned embodiments, the composition is a solution including water and alcohol, wherein said alcohol is at least one, two or more selected from a group consisting of methanol, ethanol, propanol, isopropanol, n-butanol, isobutanol, tert-butanol, n-amyl alcohol, isoamyl alcohol, active amyl alcohol, tert-amyl alcohol, neopentyl alcohol, methyl n-propyl carbinol, methyl isopropyl carbinol and 3-pentanol. In some embodiments of the previously mentioned embodiments, the composition is a solution including water, and ethanol and/or isopropanol.

In some embodiments of the previously mentioned embodiments, the composition is an ethanol water solution. In some embodiments of the previously mentioned embodiments, the composition is 0% to 75% (v/v) ethanol water solution. In some embodiments of the previously mentioned embodiments, the composition is 10%, 15%, 20%, 25%, 30%, 35%, 40%, 45%, 50%, 55%, 60%, 65%, 70%, or 75% (v/v) ethanol water solution. In some embodiments of the previously mentioned embodiments, the composition is 15% (v/v) ethanol water solution.

In some embodiments of the previously mentioned embodiments, in the composition, the concentration of an HPP of aspirin or other NSAIDs, such as 2-(diethylamino)ethyl 2-acetoxybenzoate or 2-(diethylamino)ethyl acetoxybenzoate hydrochloride, is about 5 mg/mL to about 500 mg/mL, in particular 5 mg/mL to 500 mg/mL. In some embodiments of the previously mentioned embodiments, in the composition, the concentration of the HPP is about 30 mg/mL to about 150 mg/mL, in particular 30 mg/mL to 150 mg/mL. In some embodiments of the previously mentioned embodiments, in the composition, the concentration of the HPP is about 50 mg/mL to about 100 mg/mL, in particular 50 mg/mL to 100 mg/mL. In some embodiments of the previously mentioned embodiments, in the composition, the concentration of the HPP is about 60 mg/mL to about 90 mg/mL, in particular 60 mg/mL to 90 mg/mL. In some embodiments of the previously mentioned embodiments, in the composition, the concentration of the HPP is about 75 mg/mL to about 85 mg/mL, in particular 75 mg/mL to 85 mg/mL. In some embodiments of the previously mentioned embodiments, in the composition, the concentration of the HPP is about 79 mg/mL, in particular 79 mg/mL.

In some embodiments of the previously mentioned embodiments, the volume of the composition in the unit dose is about 0.01 mL to about 1 mL, in particular 0.01 mL to 1 mL. In some embodiments of the previously mentioned embodiments, the volume of the composition in the unit dose is about 0.03 mL to about 0.3 mL, in particular 0.03 mL to 0.3 mL. In some embodiments of the previously mentioned embodiments, the volume of the composition in the unit dose is about 0.05 mL to about 0.2 mL, in particular 0.05 mL to 0.2 mL. In some embodiments of the previously mentioned embodiments, the volume of the composition in the unit dose is about 0.05 mL to about 0.15 mL, in particular 0.05 mL to 0.15 mL. In some embodiments of the previously mentioned embodiments, the volume of the composition in the unit dose is about 0.1 mL, in particular 0.1 mL.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is administered in an amount of about 0.1 mg to about 20 mg, in particular 0.1 mg to 20 mg per unit dose. In some embodiments of the previously mentioned embodiments, the HPP is administered in an amount of about 1 mg to about 18 mg, in particular 1 mg to 18 mg per unit dose. In some embodiments of the previously mentioned embodiments, the HPP is administered in an amount of about 3 mg to about 16 mg, in particular 3 mg to 16 mg per unit dose. In some embodiments of the previously mentioned embodiments, the HPP is administered in an amount of about 5 mg to about 15 mg, in particular 5 mg to 15 mg per unit dose. In some embodiments of the previously mentioned embodiments, the HPP is administered in an amount of about 6 mg to about 12 mg, in particular 6 mg to 12 mg per unit dose. In some embodiments of the previously mentioned embodiments, the HPP is administered in an amount of about 7 mg to about 10 mg, in particular 7 mg to 10 mg per unit dose. In some embodiments of the previously mentioned embodiments, the HPP is administered in an amount of about 7.5 mg to about 9 mg, in particular 7.5 mg to 9 mg per unit dose.

In some embodiments of the previously mentioned embodiments, one or more of the unit doses is topically administered to the subject in a single dose, wherein the one or more of unit doses are selected from a group consisting of 1 unit dose, 2 unit doses, 3 unit doses, 4 unit doses, 5 unit doses, 6 unit doses, 7 unit doses, 8 unit doses, 9 unit doses, 10 unit doses, 11 unit doses, 12 unit doses, 13 unit doses, 14 unit doses, 15 unit doses, 16 unit doses, 17 unit doses, 18 unit doses, 19 unit doses, 20 unit doses, 21 unit dose, 22 unit doses, 23 unit doses, 24 unit doses, 25 unit doses, 26 unit doses, 27 unit doses, 28 unit doses, 29 unit doses, 30 unit doses, 31 unit doses, 32 unit doses, 33 unit doses, 34 unit doses, 35 unit doses, 36 unit doses, 37 unit doses, 38 unit doses, 39 unit doses, 40 unit doses, 41 unit doses, 42 unit doses, 43 unit doses, 44 unit doses, 45 unit doses, 46 unit doses, 47 unit doses, 48 unit doses, 49 unit doses, and 50 unit doses. In some embodiments of the previously mentioned embodiments, 5-30 unit doses is topically administered to the subject in a single dose. In some embodiments of the previously mentioned embodiments, 10-20 unit doses is topically administered to the subject in a single dose. In some embodiments of the previously mentioned embodiments, 10 unit doses is topically administered to the subject in a single dose. In some embodiments of the previously mentioned embodiments, 15 unit doses is topically administered to the subject in a single dose. In some embodiments of the previously mentioned embodiments, 20 unit doses is topically administered to the subject in a single dose. In some embodiments of the previously mentioned embodiments, 25 unit doses is topically administered to the subject in a single dose. In some embodiments of the previously mentioned embodiments, 30 unit doses is topically administered to the subject in a single dose.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is administered by a spray capable of spraying about 0.1 mg to about 20 mg, in particular 0.1 mg to 20 mg, of the HPP, per spray. In some embodiments of the previously mentioned embodiments, the HPP is administered by a spray capable of spraying about 0.5 mg to about 18 mg, in particular 0.5 mg to 18 mg per spray; about 1 mg to about 16 mg, in particular 1 mg to 16 mg per spray; about 2 mg to about 14 mg, in particular 2 mg to 14 mg per spray; about 3 mg to about 12 mg, in particular 3 mg to 12 mg per spray; about 4 mg to about 10 mg, in particular 4 mg to 10 mg per spray; about 5 mg to about 9 mg, in particular 5 mg to 9 mg per spray; about 7 mg to about 8 mg, in particular 7 mg to 8 mg per spray.

In some embodiments of the previously mentioned embodiments, the dosage form is a spray. In some embodiments of the previously mentioned embodiments, the volume of the composition per spray is about 0.01 mL to about 1 mL, in particular 0.01 mL to 1 mL. In some embodiments of the previously mentioned embodiments, the volume of the composition per spray is about 0.03 mL to about 0.3 mL, in particular 0.03 mL to 0.3 mL. In some embodiments of the previously mentioned embodiments, the volume of the composition per spray is about 0.05 mL to about 0.2 mL, in particular 0.05 mL to 0.2 mL. In some embodiments of the previously mentioned embodiments, the volume of the composition per spray is about 0.07 mL to about 0.15 mL, in particular 0.07 mL to 0.15 mL. In some embodiments of the previously mentioned embodiments, the volume of the composition per spray is about 0.1 mL, in particular 0.1 mL.

In some embodiments of the previously mentioned embodiments, the drug strength per spray is between 0.1 mg to 50 mg of free base of an HPP of aspirin or other NSAIDs, such as 2-(diethylamino)ethyl 2-acetoxybenzoate or 2-(diethylamino)ethyl acetoxybenzoate hydrochloride. In some embodiments of the previously mentioned embodiments, the drug strength per spray is about 1 mg to about 20 mg, in particular 1 mg to 20 mg, sometimes preferably 3 mg to 10 mg, for example, 5 mg to 8 mg, or 6 mg to 7 mg of free base of an HPP of aspirin or other NSAIDs, such as 2-(diethylamino)ethyl 2-acetoxybenzoate or 2-(diethylamino)ethyl acetoxybenzoate hydrochloride.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is administered by a drop capable of dropping about 0.05 mg to about 20 mg, in particular 0.05 mg to 20 mg, of the HPP per drop. In some embodiments of the previously mentioned embodiments, the HPP of aspirin or other NSAIDs, such as 2-(diethylamino)ethyl 2-acetoxybenzoate or 2-(diethylamino)ethyl acetoxybenzoate hydrochloride, is administered by a drop capable of dropping about 0.1 mg to about 10 mg, in particular 0.1 mg to 10 mg per drop; about 0.2 mg to about 7 mg, in particular 0.2 mg to 7 mg per drop; especially about 0.2 mg to about 1 mg, in particular 0.2 mg to 1 mg, of the HPP per drop.

In some embodiments of the previously mentioned embodiments, the dosage form is a drop. In some embodiments of the previously mentioned embodiments, the volume of the composition per drop is about 0.01 mL to about 1 mL, in particular 0.01 mL to 1 mL. In some embodiments of the previously mentioned embodiments, the volume of the composition per drop is about 0.02 mL to about 0.3 mL, in particular 0.02 mL to 0.3 mL. In some embodiments of the previously mentioned embodiments, the volume of the composition per drop is about 0.03 mL to about 0.1 mL, in particular 0.03 mL to 0.1 mL.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is administered by a patch capable of administering about 1 mg to about 10 g, in particular 1 mg to 10 g, of 2 the HPP per patch. In some embodiments of the previously mentioned embodiments, the HPP is administered by a patch capable of administering about 50 mg to about 1 g, in particular 50 mg to 1 g per patch. In some embodiments of the previously mentioned embodiments, the HPP is administered by a patch capable of administering about 100 mg to about 500 mg, in particular 100 mg to 500 mg per patch. In some embodiments of the previously mentioned embodiments, the HPP is administered by a patch capable of administering about 200 mg to about 300 mg, in particular 200 mg to 300 mg per patch.

In some embodiments of the previously mentioned embodiments, the dosage form is a patch. In some embodiments of the previously mentioned embodiments, the volume of the composition per patch is about 0.01 mL to about 30 mL, in particular 0.01 mL to 30 mL. In some embodiments of the previously mentioned embodiments, the volume of the composition per patch is about 0.1 mL to about 10 mL, in particular 0.1 mL to 10 mL. In some embodiments of the previously mentioned embodiments, the volume of the composition per patch is about 0.2 mL to about 2 mL, in particular 0.2 mL to 2 mL. In some embodiments of the previously mentioned embodiments, the volume of the composition per patch is about 0.5 mL to about 1 mL, in particular 0.5 mL to 1 mL.

In some embodiments of the previously mentioned embodiments, the drug strength per patch is selected from a group consisting of 1.0 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 550 mg, 600 mg, 650 mg, 700 mg, 750 mg, 800 mg, 850 mg, 900 mg, 950 mg, 1 g, 1.1 g, 1.2 g, 1.3 g, 1.4 g, 1.5 g, 1.6 g, 1.7 g, 1.8 g, 1.9 g, 2 g, 2.1 g, 2.2 g, 2.3 g, 2.4 g, 2.5 g, 2.6 g, 2.7 g, 2.8 g, 2.9 g, 3 g, 3.1 g, 3.2 g, 3.3 g, 3.4 g, 3.5 g, 3.6 g, 3.7 g, 3.8 g, 3.9 g, 4 g, 4.1 g, 4.2 g, 4.3 g, 4.4 g, 4.5 g, 4.6 g, 4.7 g, 4.8 g, 4.9 g, 5 g, 5.1 g, 5.2 g, 5.3 g, 5.4 g, 5.5 g, 5.6 g, 5.7 g, 5.8 g, 5.9 g, 6 g, 6.1 g, 6.2 g, 6.3 g, 6.4 g, 6.5 g, 6.6 g, 6.7 g, 6.8 g, 6.9 g, 7 g, 7.1 g, 7.2 g, 7.3 g, 7.4 g, 7.5 g, 7.6 g, 7.7 g, 7.8 g, 7.9 g, 8 g, 8.1 g, 8.2 g, 8.3 g, 8.4 g, 8.5 g, 8.6 g, 8.7 g, 8.8 g, 8.9 g, 9 g, 9.1 g, 9.2 g, 9.3 g, 9.4 g, 9.5 g, 9.6 g, 9.7 g, 9.8 g, 9.9 g, and 10 g of free base of an HPP of aspirin or other NSAIDs, such as 2-(diethylamino)ethyl acetoxybenzoate.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is administered once, twice, three times, four times, five times or six times a day, or once every one, two, three, four, five, six, or seven days.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is administered once, twice, three times, four times, five times, six times, seven times or eight times a day. In some embodiments, sometimes preferably, the HPP is administered once a day, twice a day, or three times a day.

In some embodiments of the previously mentioned embodiments, an HPP of aspirin or other NSAIDs, such as 2-(diethylamino)ethyl 2-acetoxybenzoate or 2-(diethylamino)ethyl acetoxybenzoate hydrochloride, is administered once every hour or once every 4 to 16 hours. In some embodiments of the previously mentioned embodiments, the HPP is administered once every hour or once every 8 to 12 hours. In some embodiments of the previously mentioned embodiments, the HPP is administered once every hour or once every 2, 3, 4, 5, 6, 7, 8, 9, 10, 11, 12, 13, 14, 15, 16, 17, 18, 19, 20, 21, 22 or 23 hours. In some embodiments of the previously mentioned embodiments, the HPP is administered once every hour or once every 12 hours.

In some embodiments of the previously mentioned embodiments, the HPP of aspirin or other NSAIDs, such as 2-(diethylamino)ethyl 2-acetoxybenzoate or 2-(diethylamino)ethyl acetoxybenzoate hydrochloride, is administered once every hour to once every day, up to once every 7 days, or any frequency in between depending on a subject's conditions, sometimes preferably between once every hour and once every 24 hours, or any frequency in between, for example, once every 2 hours, every 3 hours, every 4 hours, every 5 hours, every 6 hours, every 7 hours, every 8 hours, every 9 hours, every 10 hours, every 11 hours, every 12 hours, every 13 hours, every 14 hours, every 15 hours, every 16 hours, every 17 hours, every 18 hours, every 19 hours, every 20 hours, every 21 hours, every 22 hours, or every 23 hours. In some embodiments, sometimes preferably, of the previously mentioned embodiments, the HPP of aspirin or other NSAIDs, such as 2-(diethylamino)ethyl 2-acetoxybenzoate or 2-(diethylamino)ethyl acetoxybenzoate hydrochloride, is administered once every 2 hours, once every 4 hours, once every 6 hours, once every 8 hours, once every 12 hours, once every 18 hours, or once every 24 hours.

In some embodiments of the previously mentioned embodiments, the HPP may be administered once every one, two, three, four, five, six, or seven days.

In some embodiments of the previously mentioned embodiments, the topical administration is administered for 1 day to lifetime. In some embodiments of the previously mentioned embodiments, the topical administration is administered for 112 to 3650 consecutive or non-consecutive days. In some embodiments of the previously mentioned embodiments, the topical administration is administered for 112 to 1825 consecutive or non-consecutive days. In some embodiments of the previously mentioned embodiments, the topical administration is administered for 112 to 1095 consecutive or non-consecutive days. In some embodiments of the previously mentioned embodiments, the topical administration is administered for 112 to 730 consecutive or non-consecutive days. In some embodiments of the previously mentioned embodiments, the topical administration is administered for 112 to 365 consecutive or non-consecutive days. In some embodiments of the previously mentioned embodiments, the topical administration is administered for 112 to 224 consecutive or non-consecutive days.

In some embodiments of the previously mentioned embodiments, the topical administration is administered for at least one or more consecutive days or non-consecutive days, for example for one or more consecutive or non-consecutive days selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 210, 220, 230, 240, 250, 260, 270, 280, 290, 300, 320, 340, 360, 380, 400, 420, 440, 460, 480, 500, 550, 600, 650, 700, 750, 800, 850, 900, 950, and 1000 days.

In some embodiments of the previously mentioned embodiments, the topical administration is administered for at least one or more consecutive years or non-consecutive years, for example, for one or more consecutive or non-consecutive yeas selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or up to a subject's lifetime.

In some embodiments of the previously mentioned embodiments, the device of the present disclosure includes a dosage form selected from one or more of transdermal patch, cream, foam, gel, lotion, ointment, paste, powder, shake lotion, solid, sponge, tape, tincture, vapor, injection, drops, rinces, spray, and solution. In some embodiments of the previously mentioned embodiments, the device of the present disclosure includes a dosage form selected from transdermal solutions, including one or more of transdermal drops, rinces and spray.

In some embodiments of the previously mentioned embodiments, the device of the present disclosure is a device capable of administrating about 0.1 mg to about 10 g, in particular 0.1 mg to 10 g, of an HPP of aspirin or other NSAIDs, such as 2-(diethylamino)ethyl 2-acetoxybenzoate or 2-(diethylamino)ethyl acetoxybenzoate hydrochloride, in each dose. In some embodiments of the previously mentioned embodiments, the device of the present disclosure is a device capable of administrating about 0.1 mg to about 1 g, in particular 0.1 mg to 1 g, of the HPP in each dose. In some embodiments of the previously mentioned embodiments, the device of the present disclosure is a device capable of administrating about 0.5 mg to about 500 mg, in particular 0.5 mg to 500 mg, of the HPP in each dose. In some embodiments of the previously mentioned embodiments, the device of the present disclosure is a device capable of administrating about 1 mg to about 300 mg, about 10 mg to about 300 mg, about 50 mg to about 300 mg, about 50 mg to about 200 mg, or about 200 mg to about 300 mg, of the HPP in each dose. In some embodiments of the previously mentioned embodiments, the device of the present disclosure is a device capable of administrating about 0.5 mg to about 30 mg, about 1 mg to about 20 mg, about 3 mg to about 10 mg, about 5 mg to about 9 mg, or about 6 mg to about 8 mg, of the HPP in each dose.

In some embodiments of the previously mentioned embodiments, the device is a spray capable of spraying about 0.1 mg to about 100 mg, in particular 0.1 mg to 100 mg, of an HPP of aspirin or other NSAIDs, such as 2-(diethylamino)ethyl 2-acetoxybenzoate or 2-(diethylamino)ethyl acetoxybenzoate hydrochloride, in each spray. In some embodiments of the previously mentioned embodiments, the device is a spray capable of spraying about 0.2 mg to about 30 mg, in particular 0.2 mg to 30 mg, of the HPP in each spray. In some embodiments of the previously mentioned embodiments, the device is a spray capable of spraying about 0.5 mg to about 16 mg, about 1 mg to about 14 mg, about 2 mg to about 12 mg, about 3 mg to about 10 mg, of the HPP in each spray. In some embodiments of the previously mentioned embodiments, the device is a spray capable of spraying about 4 mg to about 9 mg, about 5 mg to about 9 mg, or about 6 mg to about 9 mg, of the HPP in each spray. In some embodiments of the previously mentioned embodiments, the device is a spray capable of spraying about 7 mg to about 8 mg, in particular 7 mg to 8 mg, of 2-(diethylamino)ethyl acetoxybenzoate and/or a related high penetration prodrug of aspirin and/or other NSAIDs or pharmaceutically acceptable salts thereof in each spray.

In some embodiments of the previously mentioned embodiments, the device is a spray including a nozzle, the nozzle sprays about 0.1 mg to about 100 mg, in particular 0.1 mg to 100 mg, of an HPP of aspirin or other NSAIDs, such as 2-(diethylamino)ethyl 2-acetoxybenzoate or 2-(diethylamino)ethyl acetoxybenzoate hydrochloride, in each time of pressing the nozzle. In some embodiments of the previously mentioned embodiments, the nozzle sprays about 0.2 mg to about 30 mg, about 0.5 mg to about 16 mg, about 1 mg to about 14 mg, about 2 mg to about 12 mg, 3 mg to about 11 mg, about 4 mg to about 10 mg, about 5 mg to about 9 mg, or about 6 mg to about 9 mg, of the HPP in each time of pressing the nozzle. In some embodiments of the previously mentioned embodiments, the device is a spray including a nozzle, the nozzle sprays about 7 mg to about 8 mg, in particular 7 mg to 8 mg, of 2-(diethylamino)ethyl acetoxybenzoate and/or a related high penetration prodrug of aspirin and/or other NSAIDs or pharmaceutically acceptable salts thereof in each time of pressing the nozzle.

In some embodiments of the previously mentioned embodiments, the device is a drop capable of dropping about 0.01 mg to about 20 mg, in particular 0.01 mg to 20 mg, of an HPP of aspirin or other NSAIDs, such as 2-(diethylamino)ethyl 2-acetoxybenzoate or 2-(diethylamino)ethyl acetoxybenzoate hydrochloride, in each drop. In some embodiments of the previously mentioned embodiments, the device is a drop capable of dropping about 0.02 mg to about 18 mg about 0.05 mg to about 16 mg, about 0.1 mg to about 14 mg, about 0.2 mg to about 12 mg, about 0.3 mg to about 10 mg, about 0.4 mg to about 9 mg, 0.5 mg to about 8 mg, 0.7 mg to 7 mg, of the HPP in each drop. In some embodiments of the previously mentioned embodiments, the device is a drop capable of dropping about 1 mg to about 6 mg, about 2 mg to about 5 mg, about 3 mg to about 4 mg, of the HPP in each drop.

In some embodiments of the previously mentioned embodiments, the device is a patch capable of administering about 0.1 mg to about 20 g, in particular 0.1 mg to 20 g, of an HPP of aspirin or other NSAIDs, such as 2-(diethylamino)ethyl 2-acetoxybenzoate or 2-(diethylamino)ethyl acetoxybenzoate hydrochloride, in each patch. in some embodiments of the previously mentioned embodiments, the device is a patch capable of administering about 0.5 mg to about 5 g, about 1 mg to about 1000 mg, about 2 mg to about 500 mg, about 5 mg to about 400 mg, about 10 mg to about 350 mg, about 20 mg to about 300 mg, about 30 mg to about 250 mg, about 40 mg to about 200 mg, of the HPP in each patch. In some embodiments of the previously mentioned embodiments, the device is a patch capable of administering about 50 mg to about 200 mg, 60 mg to about 200 mg, about 70 mg to about 200 mg, of the HPP in each patch. In some embodiments of the previously mentioned embodiments, the device is a patch capable of administering about 100 mg to about 150 mg, in particular 100 mg to 150 mg, of the HPP in each patch.

One aspect of the invention is to increase the tissue penetration of the NSAIDs, then to reduce the plasma drug exploration to reduce the side effects and to increase the tissue drug exploration to increase the drug efficacy.

In one embodiment, a composition including 2-(diethylamino)ethyl acetoxybenzoate hydrochloride is presented as the table below.

| | Unit Formula (mg) | | |
|---|---|---|---|
| Components | Amount | Percentage | Function |
| 2-(diethylamino)ethyl | 1582 mg | 7.91% in 15% | Active |
| acetoxybenzoate hydrochloride | | ethanol | ingredient |
| Ethanol (15% in water, v/v) | 20 mL | 15% ethanol in | Diluent |
| | | water | (excipient) |

In one embodiment, a composition including 2-(diethylamino)ethyl 2′,4′-difluoro-4-acetoxy-[1,1′-biphenyl]-3-carboxylate hydrochloride is presented as the table below.

| | Unit Formula (mg) | | | |
|---|---|---|---|---|
| Components | Amount | | Percentage | Function |
| 2-(diethylamino)ethyl | 1530 | mg | 7.65% in | Active |
| 2′,4′-difluoro-4-acetoxy- | | | 35% | ingredient |
| [1,1′-biphenyl]-3- | | | ethanol | |
| carboxylate | | | | |
| hydrochloride | | | | |
| Ethanol (15% in water, | 20 | mL | 35% ethanol | Diluent |
| v/v) | | | in water | (excipient) |

In one embodiment, a composition including 2-(diethylamino)ethyl 2′,4′-difluoro-4-hydroxyl-[1,1′-biphenyl]-3-carboxylate hydrochloride is presented as the table below.

| | Unit Formula (mg) | | | |
|---|---|---|---|---|
| Components | Amount | | Percentage | Function |
| 2-(diethylamino)ethyl | 1546 | mg | 7.73% in | Active |
| 2′,4′-difluoro-4-hydroxyl- | | | 35% | ingredient |
| [1,1′-biphenyl]-3- | | | ethanol | |
| carboxylate | | | | |
| hydrochloride | | | | |
| Ethanol (35% in water, | 20 | mL | 35% ethanol | Diluent |
| v/v) | | | in water | (excipient) |

In one embodiment, a composition including 2-(diethylamino)ethyl 2-(2-acetoxybenzoyl)oxybenzoate hydrochloride is presented as the table below.

| | Unit Formula (mg) | | | |
|---|---|---|---|---|
| Components | Amount | | Percentage | Function |
| 2-(diethylamino)ethyl | 1528 | mg | 7.64% in | Active |
| 2-(2-acetoxybenzoyl)oxyben- | | | 35% | ingredient |
| zoate hydrochloride | | | ethanol | |
| Ethanol (35% in water, | 20 | mL | 35% | Diluent |
| v/v) | | | ethanol | (excipient) |
| | | | in water | |

In one embodiment, a composition including 2-(diethylamino)ethyl 2-(2-hydroxybenzoyl)oxybenzoate hydrochloride is presented as the table below.

| | Unit Formula (mg) | | | |
|---|---|---|---|---|
| Components | Amount | | Percentage | Function |
| 2-(diethylamino)ethyl | 1543 | mg | 7.71% in | Active |
| 2-(2-hydroxybenzoyl)oxyben- | | | 35% | ingredient |
| zoate hydrochloride | | | ethanol | |
| Ethanol (35% in water, | 20 | mL | 35% | Diluent |
| v/v) | | | ethanol | (excipient) |
| | | | in water | |

In one embodiment, a composition including (pyrrolidin-2-yl)methyl acetoxybenzoate is presented as the table below.

| | Unit Formula (mg) | | | |
|---|---|---|---|---|
| Components | Amount | | Percentage | Function |
| (pyrrolidin-2-yl)methyl | 1594 | mg | 7.97% in 15% | Active |
| acetoxybenzoate | | | ethanol | ingredient |
| hydrochloride | | | | |
| Ethanol (15% in water, | 20 | mL | 15% ethanol | Diluent |
| v/v) | | | in water | (excipient) |

In one embodiment, a composition including (pyrrolidin-2-yl)methyl hydroxybenzoate is presented as the table below.

| | Unit Formula (mg) | | | |
|---|---|---|---|---|
| Components | Amount | | Percentage | Function |
| (pyrrolidin-2-yl)methyl | 1631 | mg | 8.15% in | Active |
| hydroxybenzoate | | | 15% ethanol | ingredient |
| hydrochloride | | | | |
| Ethanol (15% in water, | 20 | mL | 15% ethanol | Diluent |
| v/v) | | | in water | (excipient) |

In one embodiment, a composition including (pyrrolidin-2-yl)methyl 2′,4′-difluoro-4-acetoxy-[1,1′-biphenyl]-3-carboxylate hydrochloride is presented as the table below.

| | Unit Formula (mg) | | | |
|---|---|---|---|---|
| Components | Amount | | Percentage | Function |
| (pyrrolidin-2-yl)methyl | 1536 | mg | 7.68% in | Active |
| 2′,4′-difluoro-4-acetoxy- | | | 35% | ingredient |
| [1,1′-biphenyl]-3- | | | ethanol | |
| carboxylate | | | | |
| hydrochloride | | | | |
| Ethanol (35% in water, | 20 | mL | 35% ethanol | Diluent |
| v/v) | | | in water | (excipient) |

In one embodiment, a composition including (pyrrolidin-2-yl)methyl 2′,4′-difluoro-4-hydroxyl-[1,1′-biphenyl]-3-carboxylate hydrochloride is presented as the table below.

| | Unit Formula (mg) | | | |
|---|---|---|---|---|
| Components | Amount | | Percentage | Function |
| (pyrrolidin-2-yl)methyl | 1553 | mg | 7.77% in | Active |
| 2′,4′-difluoro-4-hydroxyl- | | | 35% | ingredient |
| [1,1′-biphenyl]-3- | | | ethanol | |
| carboxylate | | | | |
| hydrochloride | | | | |
| Ethanol (35% in water, | 20 | mL | 35% ethanol | Diluent |
| v/v) | | | in water | (excipient) |

In one embodiment, a composition including (pyrrolidin-2-yl)methyl 2-(2-acetoxybenzoyl)oxybenzoate hydrochloride is presented as the table below.

| | Unit Formula (mg) | | | |
|---|---|---|---|---|
| Components | Amount | | Percentage | Function |
| (pyrrolidin-2-yl)methyl | 1533 | mg | 7.67% in | Active |
| 2-(2-acetoxybenzoyl)oxyben- | | | 35% | ingredient |
| zoate hydrochloride | | | ethanol | |
| Ethanol (35% in water, v/v) | 20 | mL | 35% | Diluent |
| | | | ethanol | (excipient) |
| | | | in water | |

In one embodiment, a composition including (pyrrolidin-2-yl)methyl 2-(2-hydroxybenzoyl)oxybenzoate hydrochloride is presented as the table below.

| | Unit Formula (mg) | | | |
|---|---|---|---|---|
| Components | Amount | | Percentage | Function |
| (pyrrolidin-2-yl)methyl | 1550 | mg | 7.75% in | Active |
| 2-(2-hydroxybenzoyl)oxyben- | | | 35% | ingredient |
| azote hydrochloride | | | ethanol | |
| Ethanol (35% in water, v/v) | 20 | mL | 35% | Diluent |
| | | | ethanol | (excipient) |
| | | | in water | |

In one embodiment, a subject will spray five sprays (e.g., 35 mg of 2-(diethylamino)ethyl acetoxybenzoate hydrochloride in 15% ethanol) of the drug solution to the skin around the neck, for once, twice, three times or four times per day until complete recovery post strokes.

In one embodiment, a subject will spray five sprays (e.g., 35 mg of 2-(diethylamino)ethyl acetoxybenzoate hydrochloride in 15% ethanol) of the drug solution to the skin around the neck, chest, back, abdomen, head, arms, hands, legs, feet, and other part, for once, twice, three times or four times per day until complete recovery from atherosclerosis.

In one embodiment, a subject will spray five sprays (e.g., 35 mg of 2-(diethylamino)ethyl acetoxybenzoate hydrochloride in 15% ethanol) of the drug solution to the skin around the chest, for once, twice, three times or four times per day until complete recovery post heart attacks.

In one embodiment, a subject will spray five sprays (e.g., 35 mg of 2-(diethylamino)ethyl acetoxybenzoate hydrochloride in 15% ethanol) of the drug solution to the skin around the chest, for once, twice, three times or four times per day until complete recovery from heart failure.

In one embodiment, a subject will spray five sprays (e.g., 35 mg of 2-(diethylamino)ethyl acetoxybenzoate hydrochloride in 15% ethanol) of the drug solution to the skin around the chest, for once, twice, three times or four times per day until complete recovery from coronary artery disease.

In one embodiment, a subject will spray five sprays (e.g., 35 mg of 2-(diethylamino)ethyl acetoxybenzoate hydrochloride in 15% ethanol) of the drug solution to the skin around the chest, for once, twice, three times or four times per day until complete recovery from angina.

In one embodiment, a subject will spray five sprays (e.g., 35 mg of 2-(diethylamino)ethyl acetoxybenzoate hydrochloride in 15% ethanol) of the drug solution to the skin around the chest, for once, twice, three times or four times per day until complete recovery from bad heart rhythms.

In one embodiment, a subject will spray five sprays (e.g., 35 mg of 2-(diethylamino)ethyl acetoxybenzoate hydrochloride in 15% ethanol) of the drug solution to the skin around the chest, for once, twice, three times or four times per day until complete recovery from cardiovascular diseases.

In one embodiment, a subject will spray 10-30 sprays (e.g., 70-210 mg of 2-(diethylamino)ethyl acetoxybenzoate hydrochloride in 15% ethanol) of the drug solution to the skin around the neck, back, chest, legs, arms, abdomen, hands, feet, head and other parts, for once, twice, three times or four times per day until complete recovery post strokes.

In one embodiment, a subject will spray 10-30 sprays (e.g., 70-210 mg of 2-(diethylamino)ethyl acetoxybenzoate hydrochloride in 15% ethanol) of the drug solution to the skin around the neck, chest, back, abdomen, head, arms, hands, legs, feet, and other part, for once, twice, three times or four times per day until complete recovery from atherosclerosis.

In one embodiment, a subject will spray 10-30 sprays (e.g., 70-210 mg of 2-(diethylamino)ethyl acetoxybenzoate hydrochloride in 15% ethanol) of the drug solution to the skin around the chest, neck, back, abdomen, and other parts, for once, twice, three times or four times per day until complete recovery post heart attacks.

In one embodiment, a subject will spray 10-30 sprays (e.g., 70-210 mg of 2-(diethylamino)ethyl acetoxybenzoate hydrochloride in 15% ethanol) of the drug solution to the skin around the chest, neck, back, abdomen, legs, arms, and other parts, for once, twice, three times or four times per day until complete recovery from heart failure.

In one embodiment, a subject will spray 10-30 sprays (e.g., 70-210 mg of 2-(diethylamino)ethyl acetoxybenzoate hydrochloride in 15% ethanol) of the drug solution to the skin around the chest, neck, back, abdomen, legs, arms, and other parts, for once, twice, three times or four times per day until complete recovery from coronary artery disease.

In one embodiment, a subject will spray 10-30 sprays (e.g., 70-210 mg of 2-(diethylamino)ethyl acetoxybenzoate hydrochloride in 15% ethanol) of the drug solution to the skin around the chest, neck, back, abdomen, legs, arms, and other parts, for once, twice, three times or four times per day until complete recovery from angina.

In one embodiment, a subject will spray 10-30 sprays (e.g., 70-210 mg of 2-(diethylamino)ethyl acetoxybenzoate hydrochloride in 15% ethanol) of the drug solution to the skin around the chest, neck, back, abdomen, legs, arms, and other parts, for once, twice, three times or four times per day until complete recovery from bad heart rhythms.

In one embodiment, a subject will spray 10-30 sprays (e.g., 70-210 mg of 2-(diethylamino)ethyl acetoxybenzoate hydrochloride in 15% ethanol) of the drug solution to the skin around the chest, neck, back, abdomen, legs, arms, and other parts, for once, twice, three times or four times per day until complete recovery from cardiovascular diseases.

### EXAMPLES

The following non-limiting examples will further illustrate certain aspects of the present invention.

### Example 1 Preparation of 2-(diethylamino)ethyl Acetoxybenzoate Hydrochloride (an HPP of Aspirin)

Acetoxybenzoyl chloride (20 g) was dissolved in ethyl acetate (100 ml). The mixture was cooled to 0° C. Diethylaminoethanol (14 g) was added into the reaction mixture. The mixture was stirred for 3 hours at RT, and then washed with water (5×30 ml). 3N HCl in ethanol (30 ml) was added, the solid was collected and washed with ethyl acetate (5×50 ml) and dried in vacuum oven at 40° C. (90% yield).

### Example 2 Preparation of 2-(diethylamino)ethyl Hydroxybenzoate Hydrochloride (an HPP of Salicylic Acid)

2-(Diethylamino)ethyl acetoxybenzoate hydrochloride (20 g) was dissolved in water (100 ml) and 3N HCl (10 ml). The mixture was stirred for overnight at RT. Ethyl acetate (300 ml) was added into the mixture, the pH of the mixture was adjusted to 8 with solid NaHCO₃, the ethyl acetate layer was collected and washed with water (3×), 1N HCl in ethyl acetate (100 ml) was added, the solid was collected and washed with ethyl acetate (5×20 ml) and dried in vacuum oven at 40° C. (72% yield).

### Example 3 Preparation of 2-(diethylamino)ethyl 5-(2,4-Difluorophenyl) Acetoxybenzoate Hydrochloride an HPP of Acetyldiflunisal)

5-(2,4-Difluorophenyl) acetoxybenzoyl chloride (2′,4′-difluoro-4-hydroxy-[1,1′-biphenyl]-3-carbonyl chloride) (31 g) was dissolved in ethyl acetate (100 ml). The mixture was cooled to 0° C. Diethylaminoethanol (14 g) was added into the reaction mixture. The mixture was stirred for 3 hours at RT. Ethyl acetate mixture was washed with water (5×30 ml). 3N HCl in ethanol (30 ml) was added, the solid was collected and washed with ethyl acetate (5×50 ml) and dried in vacuum oven at 40° C. (88% yield).

### Example 4 Preparation of 2-(diethylamino)ethyl 2′,4′-difluoro-4-hydroxyl-[1,1′-biphenyl]-3-carboxylate Hydrochloride (an HPP of Diflunisal)

2′,4′-Difluoro-4-hydroxy-[1,1′-biphenyl]-3-carboxylate hydrochloride (20 g) was dissolved in water (100 ml) and 3N HCl (10 ml). The mixture was stirred for overnight at RT. Ethyl acetate (300 ml) was added into the mixture, the pH of the mixture was adjusted to 8 with solid NaHCO₃, the ethyl acetate layer was collected and washed with water (3×), 1N HCl in ethyl acetate (100 ml) was added, the solid was collected and washed with ethyl acetate (5×20 ml) and dried in vacuum oven at 40° C. (80% yield).

### Example 5 Preparation of (pyrrolidin-2-yl)methyl Acetoxybenzoate Hydrochloride (an HPP of Aspirin)

Acetylsalicylic acid (18 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (50 g in 250 ml of water) (2×250 ml), and water (3×300 ml). The solution was dried over sodium sulfate. Sodium sulfate was removed by filtration, the filtrate was washed with ethyl acetate (3×50 ml), and the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (85% yield).

### Example 6 Preparation of (pyrrolidin-2-yl)methyl Hydroxybenzoate Hydrochloride

(Pyrrolidin-2-yl)methyl acetoxybenzoate hydrochloride (20 g) was dissolved in water (100 ml) and 3N HCl (10 ml). The mixture was stirred for overnight at RT. Ethyl acetate (300 ml) was added into the mixture, the pH of the mixture was adjusted to 8 with solid NaHCO₃, the ethyl acetate layer was collected and washed with water (3×), 1N HCl in ethyl acetate (100 ml) was added, the solid was collected and washed with ethyl acetate (5×20 ml) and dried in vacuum oven at 40° C. (80% yield).

### Example 7 Preparation of (pyrrolidin-2-yl)methyl 2′,4′-difluoro-4-acetoxy-[1,1′-biphenyl]-3-carboxylate Hydrochloride

2′,4′-Difluoro-4-acetoxy-[1,1′-biphenyl]-3-carboxylic acid (31 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (50 g in 250 ml of water) (2×250 ml), and water (3×300 ml). The solution was dried over sodium sulfate. Sodium sulfate was removed by filtration, and the filtrate was washed with ethyl acetate (3×50 ml), and the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (85% yield).

### Example 8 Preparation of (pyrrolidin-2-yl)methyl 2′,4′-difluoro-4-hydroxyl-[1,1′-biphenyl]-3-carboxylate Hydrochloride

(Pyrrolidin-2-yl)methyl 2′,4′-difluoro-4-acetoxy-[1,1′-biphenyl]-3-carboxylate hydrochloride (20 g) was dissolved in water (100 ml) and 3N HCl (10 ml). The mixture was stirred for overnight at RT. Ethyl acetate (300 ml) was added into the mixture, the pH of the mixture was adjusted to 8 with solid NaHCO₃, the ethyl acetate layer was collected and washed with water (3×100 ml), 1N HCl in ethyl acetate (100 ml) was added, the solid was collected and washed with ethyl acetate (5×20 ml) and dried in vacuum oven at 40° C. (80% yield).

### Example 9 Preparation of 2-(diethylamino)ethyl 2-(2-acetoxybenzoyl)oxybenzoate Hydrochloride (a Prodrug of Acetylsalsalate)

2-(2-Acetoxybenzoyl)oxybenzoyl chloride (28 g) was dissolved in ethyl acetate (100 ml). The mixture was cooled to 0° C. Diethylaminoethanol (14 g) was added into the reaction mixture. The mixture was stirred for 3 hours at RT. Ethyl acetate mixture was washed with water (5×30 ml). 3N HCl in ethanol (30 ml) was added, the solid was collected and washed with ethyl acetate (5×50 ml) and dried in vacuum oven at 40° C. (91% yield).

### Example 10 Preparation of 2-(diethylamino)ethyl 2-(2-hydroxybenzoyl)oxybenzoate Hydrochloride (a Prodrug Of Salsalate)

2-(Diethylamino)ethyl 2-(2-acetoxybenzoyl)oxybenzoate hydrochloride (20 g) was dissolved in water (100 ml) and 3N HCl (10 ml). The mixture was stirred for overnight at RT. Ethyl acetate (300 ml) was added into the mixture, the pH of the mixture was adjusted to 8 with solid NaHCO₃, the ethyl acetate layer was collected and washed with water (3×100 ml), 1N HCl in ethyl acetate (100 ml) was added, the solid was collected and washed with ethyl acetate (5×20 ml) and dried in vacuum oven at 40° C. (68% yield).

### Example 11 Preparation of (pyrrolidin-2-yl)methyl 2-(2-acetoxybenzoyl)oxybenzoate Hydrochloride

2-(2-Acetoxybenzoyl)oxybenzoic acid (28 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water) (2×250 ml), and water (3×300 ml). The solution was dried over sodium sulfate. Sodium sulfate was removed by filtration, and the filtrate was washed with ethyl acetate (3×50 ml), and the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (85% yield).

### Example 12 Preparation of (pyrrolidin-2-yl)methyl 2-(2-hydroxybenzoyl)oxybenzoate Hydrochloride

(Pyrrolidin-2-yl)methyl 2-(2-acetoxybenzoyl)oxybenzoate hydrochloride (20 g) was dissolved in water (100 ml) and 3N HCl (10 ml). The mixture was stirred for overnight at RT. Ethyl acetate (300 ml) was added into the mixture, the pH of the mixture was adjusted to 8 with solid NaHCO₃, the ethyl acetate layer was collected and washed with water (3×100 ml), 1N HCl in ethyl acetate (100 ml) was added, the solid was collected and washed with ethyl acetate (5×20 ml) and dried in vacuum oven at 40° C. (80% yield).

### Example 13 the Skin Penetration Rates of HPPs

The penetration rates of 2-(diethylamino)ethyl acetoxybenzoate hydrochloride (Compound-1), (pyrrolidin-2-yl)methyl acetoxybenzoate hydrochloride (Compound-2), Aspirin (compound-3), 2-(diethylamino)ethyl hydroxybenzoate hydrochloride (Compound-4), 2 (pyrrolidin-2-yl)methyl hydroxybenzoate hydrochloride (Compound-5), Salicylic acid (Compound-6), 2-(diethylamino)ethyl 2′,4′-difluoro-4-acetoxy-[1,1′-biphenyl]-3-carboxylate hydrochloride (Compound-7), (pyrrolidin-2-yl)methyl 2′,4′-difluoro-4-acetoxy-[1,1′-biphenyl]-3-carboxylate hydrochloride (Compound-8), 2′,4′-difluoro-4-acetoxy-[1,1′-biphenyl]-3-carboxylic acid (Compound-9), 2-(diethylamino)ethyl 2′,4′-difluoro-4-hydroxyl-[1,1′-biphenyl]-3-carboxylate hydrochloride (Compound-10), (pyrrolidin-2-yl)methyl 2′,4′-difluoro-4-hydroxyl-[1,1′-biphenyl]-3-carboxylate hydrochloride (Compound-11), 2′,4′-difluoro-4-hydroxyl-[1,1′-biphenyl]-3-carboxylic acid (diflunisal, Compound-12), 2-(diethylamino)ethyl 2-(2-acetoxybenzoyl)oxybenzoate hydrochloride (Compound-13), (pyrrolidin-2-yl)methyl 2-(2-acetoxybenzoyl)oxybenzoate hydrochloride (Compound-14), 2-(2-acetoxybenzoyl)oxybenzoic acid (acetylsalsalate, Compound-15), 2-(diethylamino)ethyl 2-(2-hydroxybenzoyl)oxybenzoate hydrochloride (Compound-16) (pyrrolidin-2-yl)methyl 2-(2-hydroxybenzoyl)oxybenzoate hydrochloride (Compound-17) and 2-(2-hydroxybenzoyl)oxybenzoic acid (Salsalate, Compound-18) through rabbit skin were measured in vitro by using modified Franz cells, which were isolated from rabbit skin tissue (300-350 μm thick) of the back areas. The receiving fluid consisted of 10 ml of pure water are shown in Table 1. The results suggest that the transportational unit has a very important role in the passage of the drug across the membrane and skin barrier.

TABLE 1
The Cumulative amounts of anti-inflammatory
drugs in a period of 8 hours.

| | The cumulative amount of test compounds | | | | | | | |
|---|---|---|---|---|---|---|---|---|
| | 1 h | | 2 h | | 4 h | | 8 h | |
| Compound-1 | 0.61 | mg | 1.27 | mg | 2.58 | mg | 5.19 | mg |
| Compound-2 | 0.52 | mg | 1.16 | mg | 2.37 | mg | 4.78 | mg |
| Compound-3 | 0.000 | mg | 0.001 | mg | 0.002 | mg | 0.003 | mg |
| Compound-4 | 0.57 | mg | 1.19 | mg | 2.36 | mg | 4.79 | mg |
| Compound-5 | 0.49 | mg | 1.07 | mg | 2.17 | mg | 4.28 | mg |
| Compound-6 | 0.000 | mg | 0.001 | mg | 0.002 | mg | 0.002 | mg |
| Compound-7 | 0.53 | mg | 1.07 | mg | 2.16 | mg | 4.33 | mg |
| Compound-8 | 0.45 | mg | 0.98 | mg | 2.02 | mg | 4.11 | mg |
| Compound-9 | 0.000 | mg | 0.000 | mg | 0.001 | mg | 0.002 | mg |
| Compound-10 | 0.49 | mg | 1.03 | mg | 2.12 | mg | 4.39 | mg |
| Compound-11 | 0.43 | mg | 0.99 | mg | 2.01 | mg | 4.05 | mg |
| Compound-12 | 0.000 | mg | 0.001 | mg | 0.001 | mg | 0.003 | mg |
| Compound-13 | 0.46 | mg | 0.97 | mg | 1.99 | mg | 3.89 | mg |
| Compound-14 | 0.37 | mg | 0.84 | mg | 1.82 | mg | 3.69 | mg |
| Compound-15 | 0.000 | mg | 0.000 | mg | 0.001 | mg | 0.001 | mg |
| Compound-16 | 0.37 | mg | 0.87 | mg | 1.71 | mg | 3.56 | mg |
| Compound-17 | 0.32 | mg | 0.68 | mg | 1.39 | mg | 2.97 | mg |
| Compound-18 | 0.000 | mg | 0.000 | mg | 0.001 | mg | 0.001 | mg |

### Example 14 Efficacy of 2-(diethylamino)ethyl Acetoxybenzoate Hydrochloride in Rats with Acute Ischemia Induced by Middle Cerebral Artery Occlusion (MCAO)

The objective of this study was to investigate therapeutic efficacy of 2-(diethylamino)ethyl acetoxybenzoate hydrochloride for brain ischemic injury and associated neurological deficits induced by a temporal MCAO in rat.

Male SD rats (258-280 g) were used. The rats were selected for inclusion based upon acceptable clinical condition and body weight. Animals were randomly assigned to 7 groups (25 rats/group for MCAO surgery including transdermal 2-(diethylamino)ethyl acetoxybenzoate hydrochloride, oral aspirin, MCI-186/Edaravone (i.v) treatment and vehicle groups; 8 rats/group for sham surgery group). The rats were kept one week acclamation in the 15 animal facility prior to any procedures. Animal identification number was labeled on the tail and cage tag as well. See the experimental design as illustrated in Table 2.

2-(Diethylamino)ethyl acetoxybenzoate hydrochloride, aspirin, Edaravone, and vehicle were administered 1 h after MCAO surgery (Day 0), and Day 1, 2, 3, 4, 5, 6, 7, 8, 9, 10, 11, 12, 13, and 14.

TABLE 2
Experimental Design

| | | | Dosing | | | Duration |
|---|---|---|---|---|---|---|
| Group | Treatment | N | Level | Route | Regimen | (days) |
| 1 | Sham | 8 | 0 mg/kg | topical | Bid | 14 |
| | operation | | | | | |
| 2 | Vehicle, | 25 | 0 mg/kg, | topical | Bid | 14 |
| | 15% ethanol | | | | | |
| 3 | MCI-186 | 25 | 3 mg/kg | i.v. | q.d. | 14 |
| | (Edaravone) | | | | | |
| 4 | Aspirin | 25 | 60 mg/kg | oral | Bid | 14 |
| 5 | Low dose | 25 | 15 mg/kg, | topical | Bid | 14 |
| 6 | Middle dose | 25 | 30 mg/kg, | topical | Bid | 14 |
| 7 | High dose | 25 | 60 mg/kg, | topical | Bid | 14 |

Before MCAO the animals were fasting overnight but allowed free access to water. Under anesthesia, the right common carotid artery (CCA), internal carotid artery (ICA), and external carotid artery (ECA) were exposed through a midline incision of the neck. A commercial monofilament (silicon-coated) was used as an occluder and inserted via the CCA. The occluder was advanced into the CCA 18±0.5 mm beyond the carotid bifurcation. Mild resistance indicated that the occluder was properly lodged in the anterior cerebral artery thus blocking the blood flow to the middle cerebral artery (MCA). After 1 hour, reperfusion was allowed by withdrawing the monofilament totally. Body temperature was kept around 36.5° C. with a heading pad during the surgery process. Clinical signs were tested at 2 hours after occlusion by the observer blinded to the treatment group to confirm neurological deficit. The neurological deficits (see Table 3) were evaluated daily afterwards until Day 14. Fourteen days after MCAO, animals were euthanized and the brains were cut into 5 coronal sections (2 mm in thickness by use of a rat brain matrix). The fresh brain sections were stained with 2% solution of triphenyltetrazolium chloride (TTC) at 37° C. and then fixed with 4% paraformaldehyde. Pictures of all sections were taken with a digital camera. These digital pictures were put into a computer. After a blind measurement of infarct area (%) for each section with Image-Pro Plus software, the infarct volumes per brain were obtained. The infarct area (%)=(contralateral hemisphere area—ipsilateral non-infarct area)/contralateral hemisphere area, then the issue swelling and atrophy were corrected with this formula.

TABLE 3
The score of neurological signs

| | Deficit |
|---|---|
| Item | Score |
| no neurological deficit | 0 |
| failure to extend right forepaw fully | 1 |
| inconstant circling to the right | 2 |
| constant circling to the right | 3 |
| falling to the right | 4 |
| no spontaneous walking with depressed level of consciousness | 5 |
| death | 6 |

For infarct area analysis, One-way analysis of variance (One-Way ANOVA) followed by Dunnett Multiple Comparisons Test was used for comparing the differences between treatment groups and Vehicle group. T-test was used to compare the difference between Positive control and Vehicle group. P<0.05 was considered as significant differences between the groups. Data was presented as Mean±SEM. For body weight analysis, t-test was used to compare the differences among groups in difference days after surgery.

All animals showed good general conditions before study. During this experiment mortalities were 5 of 25 (20%) in the vehicle group, 6 of 25 (24%) in the Edaravone-treated group, 3 of 25 (12%) in oral aspirin group, 1 of 25 (4%) in low dose group of the transdermal 2-(diethylamino)ethyl acetoxybenzoate hydrochloride, 0 of 25 in the middle dose group and high dose group of the transdermal 2-(diethylamino)ethyl acetoxybenzoate hydrochloride and 0 of 8 (0%) in the sham operation group. Most of the animal death occurred between 48 hours (6 rats) and 72 hours after MCAO surgery. All other MCAO animals lost weight during Day 1 till Day 4 after surgery, and then remained in a relatively stable health condition before euthanized. Animals of sham operation group only lost weight during first 2 days after sham-surgery then gradually caught back to normal level.

Few obvious bleeding animals during surgery were abandoned. Animals were monitored carefully until consciousness was fully recovered after the last step of surgery (the occluder was removed).

The rats treated with transdermal 2-(diethylamino)ethyl acetoxybenzoate hydrochloride showed less infarct volumes: low dose group (32.07%±2.061, P<0.05), middle dose group (27.11%±1.658, P<0.01), high dose group (25.15%=2.001, P<0.01), when compared with i.v. Edaravone (38.07%±2.031), oral aspirin (36.27%±2.123) and the vehicle group (40.53%±2.378), expressed as mean±SEM. The differences of all low dose, middle dose and high dose vs. Vehicle group were statistically significant (P<0.05 or 0.01). In contrast, Aspirin and MCI-186/Edaravone-treated rats demonstrated little reduction of infarct volumes. The rats in the sham group showed an unavoidable measurement variation (2.85%±0.779). Most infarction in the vehicle-, aspirin-, and MCI-186/Edaravone-treated groups was involved in the striatum and fronto-parietal cortex that were typically supplied by the middle cerebral artery, while the infarct size in low, middle, and high dose groups were smaller with less involvement of the striatum and the corresponding cortex. The detailed analysis results of infarct volume are shown in FIG. 1.

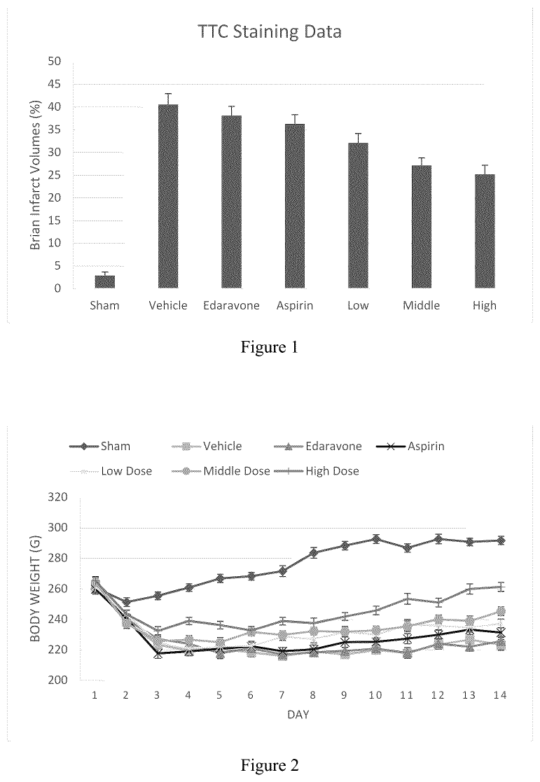

Body weight of MACO rats dropped dramatically in the first four days after surgery, and then relatively stable afterwards. While for the body weight of low (P<0.05), middle (P<0.01) and high (P<0.01) dose groups of 2-(diethylamino)ethyl acetoxybenzoate hydrochloride increased at Day 5-14, and showed significant difference from the vehicle group by t-test. The detailed information on assessment of body weight is shown in FIG. 2.

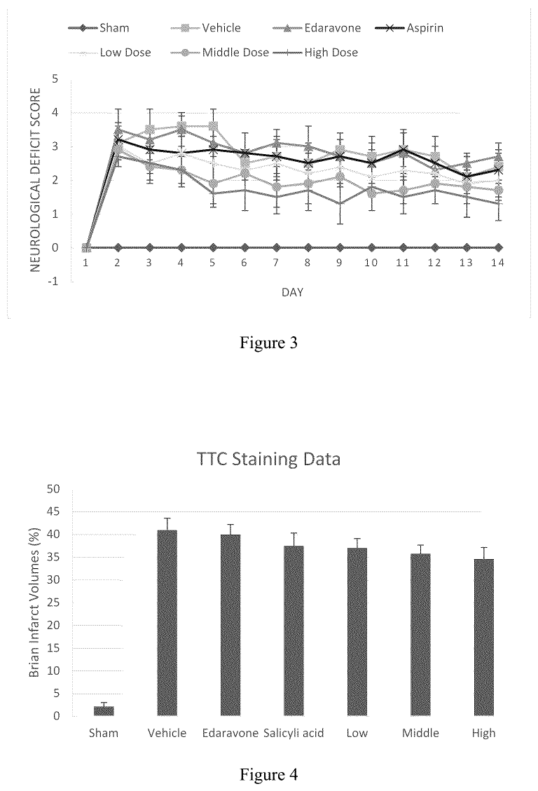

The ischemic insult caused the clinical signs of motor function impairments on the left side. The neurological deficit scores of low, middle, and high dose groups of 2-(diethylamino)ethyl acetoxybenzoate hydrochloride vs vehicle group show differences significantly. The detailed information on assessment of neurological deficit scores is shown in FIG. 3.

This study investigated therapeutic efficacy of 2-(diethylamino)ethyl acetoxybenzoate hydrochloride on cerebral ischemia in rat MCAO model. Comparing to vehicle group, low, middle, and high dose groups of 2-(diethylamino)ethyl acetoxybenzoate hydrochloride exhibited good neuroprotective effect on brain ischemic injury.

### Example 15 Efficacy of 2-(diethylamino)ethyl Hydroxybenzoate Hydrochloride in Rats with Acute Ischemia Induced by Middle Cerebral Artery Occlusion (MCAO)

This study was conducted to investigate therapeutic efficacy of 2-(diethylamino)ethyl hydroxybenzoate for brain ischemic injury and associated neurological deficits induced by a temporal MCAO in rat.

Male SD rats (258-275 g) were used. The rats were selected for inclusion based upon acceptable clinical condition and body weight. Animals were randomly assigned to 7 groups (25 rats/group for MCAO surgery including transdermal 2-(diethylamino)ethyl hydroxybenzoate hydrochloride, oral aspirin, MCI-186/Edaravone (i.v) treatment and vehicle groups; 8 rats/group for sham surgery group). The rats were kept one week acclamation in the animal facility prior to any procedures. Animal identification number was labeled on the tail and cage tag as well. See the experimental design as illustrated in Table 4.

2-(Diethylamino)ethyl hydroxybenzoate hydrochloride, salicylic acid, Edaravone, and vehicle were administered at 1 h after MCAO surgery (Day 0), and Day 1, 2, 3, 4, 5, 6, 7, 8, 9, 10, 11, 12, 13, and 14.

TABLE 4
Experimental Design

| | | | Dosing | | | Duration |
|---|---|---|---|---|---|---|
| Group | Treatment | N | Level | Route | Regimen | (days) |
| 1 | Sham | 8 | 0 mg/kg | topical | Bid | 14 |
| | operation | | | | | |
| 2 | Vehicle, | 25 | 0 mg/kg, | topical | Bid | 14 |
| | 15% ethanol | | | | | |
| 3 | MCI-186 | 25 | 3 mg/kg | i.v. | q.d. | 14 |
| | (Edaravone) | | | | | |
| 4 | salicylic acid | 25 | 60 mg/kg | oral | Bid | 14 |
| 5 | Low dose | 25 | 15 mg/kg, | topical | Bid | 14 |
| 6 | Middle dose | 25 | 30 mg/kg, | topical | Bid | 14 |
| 7 | High dose | 25 | 60 mg/kg, | topical | Bid | 14 |

Before MCAO the animals were fasting overnight but allowed free access to water. Under anesthesia, the right common carotid artery (CCA), internal carotid artery (ICA), and external carotid artery (ECA) were exposed through a midline incision of the neck. A commercial monofilament (silicon-coated) was used as an occluder and inserted via the CCA. The occluder was advanced into the CCA 18±0.5 mm beyond the carotid bifurcation. Mild resistance indicated that the occluder was properly lodged in the anterior cerebral artery thus blocking the blood flow to the middle cerebral artery (MCA). After 1 hour, reperfusion was allowed by withdrawing the monofilament totally. Body temperature was kept around 36.5° C. with a heading pad during the surgery process. Clinical signs were tested at 2 hours after occlusion by the observer blinded to the treatment group to confirm neurological deficit. The neurological deficits (see Table 3) were evaluated daily afterwards until Day 14. Fourteen days after MCAO, animals were euthanized and the brains were cut into 5 coronal sections (2 mm in thickness by use of a rat brain matrix). The fresh brain sections were stained with 2% solution of triphenyltetrazolium chloride (TTC) at 37° C. and then fixed with 4% paraformaldehyde. Pictures of all sections were taken with a digital camera. These digital pictures were put into a computer. After a blind measurement of infarct area (%) for each section with Image-Pro Plus software, the infarct volumes per brain were obtained. The infarct area (%)=(contralateral hemisphere area-ipsilateral non-infarct area)/contralateral hemisphere area, then the issue swelling and atrophy were corrected with this formula.

For infarct area analysis, One-way analysis of variance (One-Way ANOVA) followed by Dunnett Multiple Comparisons Test was used for comparing the differences between treatment groups and Vehicle group. T-test was used to compare the difference between Positive control and Vehicle group. P<0.05 was considered as significant differences between the groups. Data was presented as Mean±SEM. For body weight analysis, t-test was used to compare the differences among groups in difference days after surgery.

All animals showed good general conditions before study. During this experiment mortalities were 5 of 25 (20%) in the vehicle group, 5 of 25 (20%) in the Edaravone-treated group, 4 of 25 (16%) in oral salicylic acid group, 3 of 25 (12%) in low dose group of the transdermal 2-(diethylamino)ethyl acetoxybenzoate hydrochloride, 2 of 25 (8%) in the middle dose group of the transdermal 2-(diethylamino)ethyl hydroxybenzoate hydrochloride and 1 of 25 (4%) high dose group of the transdermal 2-(diethylamino)ethyl hydroxybenzoate hydrochloride and 0 of 8 (0%) in the sham operation group. Most of the animal death occurred between 48 hours (6 rats) and 72 hours after MCAO surgery. All other MCAO animals lost weight during Day 1 till Day 4 after surgery, and then remained in a relatively stable health condition before euthanized. Animals of sham operation group only lost weight during first 2 days after sham-surgery then gradually caught back to normal level.

Few obvious bleeding animals during surgery were abandoned. Animals were monitored carefully until consciousness was fully recovered after the last step of surgery (the occluder was removed).

The rats treated with transdermal 2-(diethylamino)ethyl hydroxybenzoate hydrochloride showed less infarct volumes: low dose group (37.01%±2.053, P<0.08), middle dose group (35.77%±1.875, P<0.07), high dose group (34.56%±2.563, P<0.05), when compared with i.v. Edaravone (39.94%±2.256), oral salicylic acid (37.47%±2.856) and the vehicle group (40.95%±2.567), expressed as mean±SEM. The differences of high dose vs. Vehicle group were statistically significant (P<0.05). In contrast, salicylic acid and MCI-186/Edaravone-treated rats demonstrated little reduction of infarct volumes. The rats in the sham group showed an unavoidable measurement variation (2.12%±0.853). Most infarction in the vehicle-, salicylic acid-, and MCI-186/Edaravone-treated groups was involved in the striatum and fronto-parietal cortex that were typically supplied by the middle cerebral artery, while the infarct size in low, middle, and high dose groups were smaller with less involvement of the striatum and the corresponding cortex. The detailed analysis results of infarct volume are shown in FIG. 4.

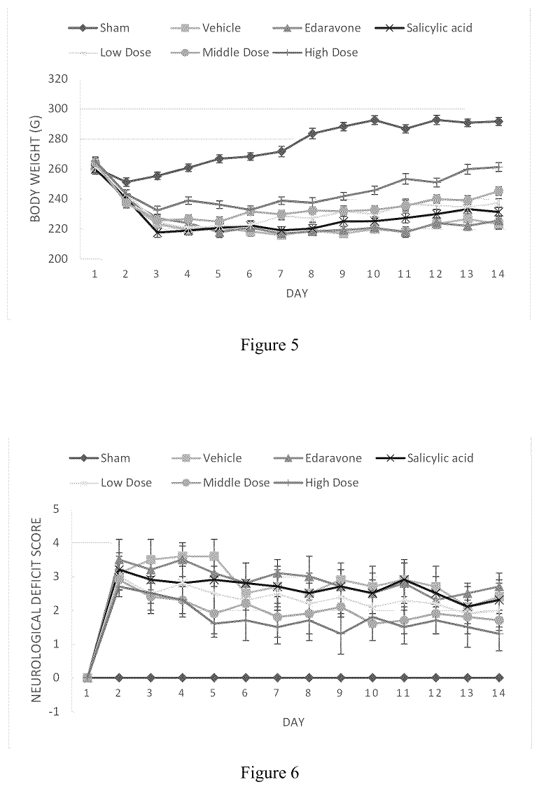

Body weight of MACO rats dropped dramatically in the first four days after surgery, and then relatively stable afterwards. While for the body weight of low (P<0.11), middle (P<0.08) and high (P<0.05) dose groups of 2-(diethylamino)ethyl hydroxybenzoate hydrochloride (a prodrug of salicylic acid) increased at Day 5-14, and showed significant difference from the vehicle group by t-test. The detailed information on assessment of body weight is shown in FIG. 5.

The ischemic insult caused the clinical signs of motor function impairments on the left side. The neurological deficit scores of high dose groups of 2-(diethylamino)ethyl hydroxybenzoate hydrochloride vs vehicle group show differences significantly. The detailed information on assessment of neurological deficit scores is shown in FIG. 6.

This study investigated therapeutic efficacy of 2-(diethylamino)ethyl hydroxybenzoate hydrochloride on cerebral ischemia in rat MCAO model. Comparing to vehicle group, high dose group of 2-(diethylamino)ethyl hydroxybenzoate hydrochloride exhibited good neuroprotective effect on brain ischemic injury.

### Example 16 Efficacy of (pyrrolidin-2-yl)methyl Acetoxybenzoate Hydrochloride in Rats with Acute Ischemia Induced by Middle Cerebral Artery Occlusion (MCAO)

The objective of this study was to investigate therapeutic efficacy of 2-(diethylamino)ethyl acetoxybenzoate for brain ischemic injury and associated neurological deficits induced by a temporal MCAO in rat.

Male SD rats (255-283 g) were used. The rats were selected for inclusion based upon acceptable clinical condition and body weight. Animals were randomly assigned to 7 groups (25 rats/group for MCAO surgery including transdermal (pyrrolidin-2-yl)methyl acetoxybenzoate hydrochloride, oral aspirin, MCI-186/Edaravone (i.v) treatment and vehicle groups; 8 rats/group for sham surgery group). The rats were kept one week acclamation in the animal facility prior to any procedures. Animal identification number was labeled on the tail and cage tag as well. See the experimental design as illustrated in Table 5.

(Pyrrolidin-2-yl)methyl acetoxybenzoate hydrochloride, aspirin, Edaravone, and vehicle were administered at 1 h after MCAO surgery (Day 0), and Day 1, 2, 3, 4, 5, 6, 7, 8, 9, 10, 11, 12, 13, and 14.

TABLE 5
Experimental Design

| | | | Dosing | | | Duration |
|---|---|---|---|---|---|---|
| Group | Treatment | N | Level | Route | Regimen | (days) |
| 1 | Sham | 8 | 0 mg/kg | topical | Bid | 14 |
| | operation | | | | | |
| 2 | Vehicle, | 25 | 0 mg/kg, | topical | Bid | 14 |
| | 15% ethanol | | | | | |
| 3 | MCI-186 | 25 | 3 mg/kg | i.v. | q.d. | 14 |
| | (Edaravone) | | | | | |
| 4 | Aspirin | 25 | 60 mg/kg | oral | Bid | 14 |
| 5 | Low dose | 25 | 15 mg/kg, | topical | Bid | 14 |
| 6 | Middle dose | 25 | 30 mg/kg, | topical | Bid | 14 |
| 7 | High dose | 25 | 60 mg/kg, | topical | Bid | 14 |

Before MCAO the animals were fasting overnight but allowed free access to water. Under anesthesia, the right common carotid artery (CCA), internal carotid artery (ICA), and external carotid artery (ECA) were exposed through a midline incision of the neck. A commercial monofilament (silicon-coated) was used as an occluder and inserted via the CCA. The occluder was advanced into the CCA 18±0.5 mm beyond the carotid bifurcation. Mild resistance indicated that the occluder was properly lodged in the anterior cerebral artery thus blocking the blood flow to the middle cerebral artery (MCA). After 1 hour, reperfusion was allowed by withdrawing the monofilament totally. Body temperature was kept around 36.5° C. with a heading pad during the surgery process. Clinical signs were tested at 2 hours after occlusion by the observer blinded to the treatment group to confirm neurological deficit. The neurological deficits (see Table 3) were evaluated daily afterwards until Day 14. Fourteen days after MCAO, animals were euthanized and the brains were cut into 5 coronal sections (2 mm in thickness by use of a rat brain matrix). The fresh brain sections were stained with 2% solution of triphenyltetrazolium chloride (TTC) at 37° C. and then fixed with 4% paraformaldehyde. Pictures of all sections were taken with a digital camera. These digital pictures were put into a computer. After a blind measurement of infarct area (%) for each section with Image-Pro Plus software, the infarct volumes per brain were obtained. The infarct area (%)=(contralateral hemisphere area-ipsilateral non-infarct area)/contralateral hemisphere area, then the issue swelling and atrophy were corrected with this formula.

For infarct area analysis, One-way analysis of variance (One-Way ANOVA) followed by Dunnett Multiple Comparisons Test was used for comparing the differences between treatment groups and Vehicle group. T-test was used to compare the difference between Positive control and Vehicle group. P<0.05 was considered as significant differences between the groups. Data was presented as Mean±SEM. For body weight analysis, t-test was used to compare the differences among groups in difference days after surgery.

All animals showed good general conditions before study. During this experiment mortalities were 5 of 25 (20%) in the vehicle group, 5 of 25 (20%) in the Edaravone-treated group, 3 of 25 (12%) in oral aspirin group, 1 of 25 (4%) in low dose group of the transdermal (pyrrolidin-2-yl)methyl acetoxybenzoate hydrochloride, 0 of 25 in the middle dose group and high dose group of the transdermal (pyrrolidin-2-yl)methyl acetoxybenzoate hydrochloride and 0 of 8 (0%) in the sham operation group. Most of the animal death occurred between 48 hours (6 rats) and 72 hours after MCAO surgery. All other MCAO animals lost weight during Day 1 till Day 4 after surgery, and then remained in a relatively stable health condition before euthanized. Animals of sham operation group only lost weight during first 2 days after sham-surgery then gradually caught back to normal level.

Few obvious bleeding animals during surgery were abandoned. Animals were monitored carefully until consciousness was fully recovered after the last step of surgery (the occluder was removed).

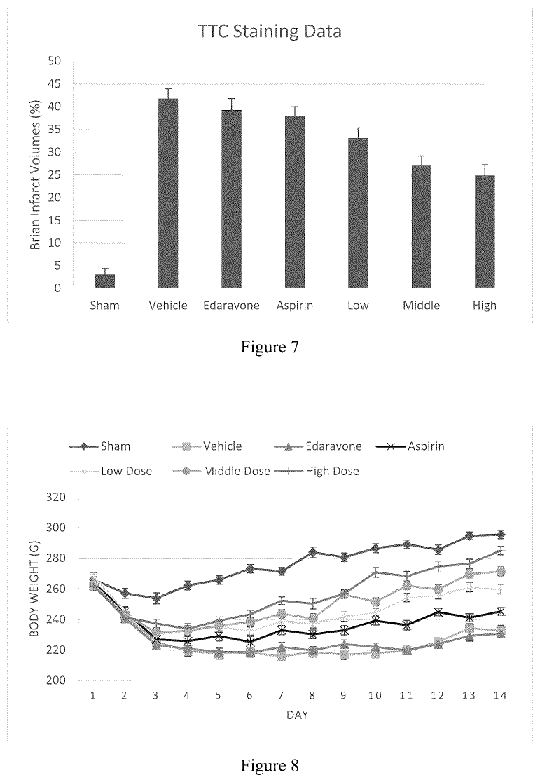

The rats treated with transdermal (pyrrolidin-2-yl)methyl acetoxybenzoate hydrochloride showed less infarct volumes: low dose group (33.11%±2.212, P<0.05), middle dose group (27.03%±2.123, P<0.01), high dose group (24.85%=2.376, P<0.01), when compared with i.v. Edaravone (39.27%±2.513), oral aspirin (37.99%±1.985) and the vehicle group (41.78%±2.179), expressed as mean±SEM. The differences of all low dose, middle dose and high dose vs. Vehicle group were statistically significant (P<0.05 or 0.01). In contrast, Aspirin and MCI-186/Edaravone-treated rats demonstrated little reduction of infarct volumes. The rats in the sham group showed an unavoidable measurement variation (3.12%±1.279). Most infarction in the vehicle-, aspirin-, and MCI-186/Edaravone-treated groups was involved in the striatum and fronto-parietal cortex that were typically supplied by the middle cerebral artery, while the infarct size in low, middle, and high dose groups were smaller with less involvement of the striatum and the corresponding cortex. The detailed analysis results of infarct volume are shown in FIG. 7.

Body weight of MACO rats dropped dramatically in the first four days after surgery, and then relatively stable afterwards. While for the body weight of low (P<0.05), middle (P<0.01) and high (P<0.01) dose groups of (pyrrolidin-2-yl)methyl acetoxybenzoate hydrochloride increased at Day 5-14, and showed significant difference from the vehicle group by t-test. The detailed information on assessment of body weight is shown in FIG. 8.

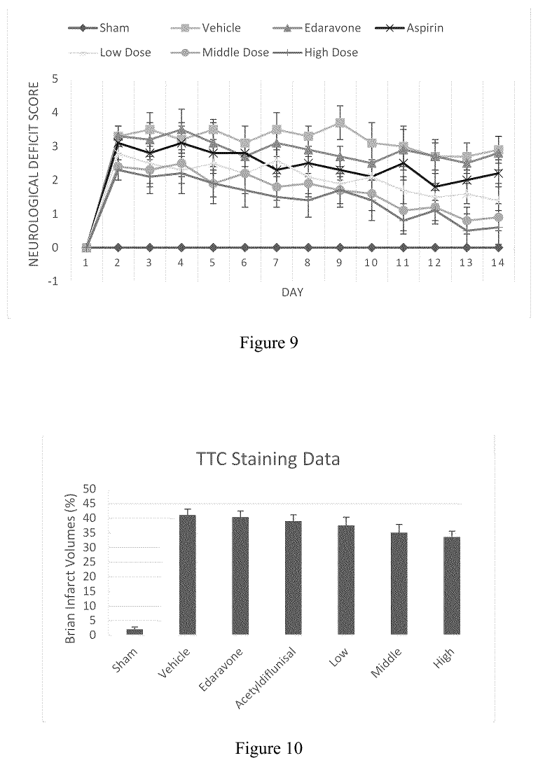

The ischemic insult caused the clinical signs of motor function impairments on the left side. The neurological deficit scores of low, middle, and high dose groups of (pyrrolidin-2-yl)methyl acetoxybenzoate hydrochloride vs vehicle group show differences significantly. The detailed information on assessment of neurological deficit scores is shown in FIG. 9.

This study investigated therapeutic efficacy of (pyrrolidin-2-yl)methyl acetoxybenzoate hydrochloride on cerebral ischemia in rat MCAO model. Comparing to vehicle group, low, middle, and high dose groups of (pyrrolidin-2-yl)methyl acetoxybenzoate hydrochloride exhibited good neuroprotective effect on brain ischemic injury.

### Example 17 Efficacy of (pyrrolidin-2-yl)methyl 2′,4′-difluoro-4-acetoxy-[1,1′-biphenyl]-3-carboxylate Hydrochloride in Rats with Acute Ischemia Induced by Middle Cerebral Artery Occlusion (MCAO)

The objective of this study was to investigate therapeutic efficacy of (pyrrolidin-2-yl)methyl 2′,4′-difluoro-4-acetoxy-[1,1′-biphenyl]-3-carboxylate hydrochloride for brain ischemic injury and associated neurological deficits induced by a temporal MCAO in rat.

Male SD rats (257-281 g) were used. The rats were selected for inclusion based upon acceptable clinical condition and body weight. Animals were randomly assigned to 7 groups (25 rats/group for MCAO surgery including transdermal (pyrrolidin-2-yl)methyl 2′,4′-difluoro-4-acetoxy-[1,1′-biphenyl]-3-carboxylate hydrochloride, acetyldiflunisal, MCI-186/Edaravone (i.v) treatment and vehicle groups; 8 rats/group for sham surgery group). The rats were kept one week acclamation in the animal facility prior to any procedures. Animal identification number was labeled on the tail and cage tag as well. See the experimental design as illustrated in Table 6.

(Pyrrolidin-2-yl)methyl 2′,4′-difluoro-4-acetoxy-[1,1′-biphenyl]-3-carboxylate hydrochloride, 2′,4′-difluoro-4-acetoxy-[1,1′-biphenyl]-carboxylic acid (acetyldiflunisal), Edaravone, and vehicle were administered at 1 h after MCAO surgery (Day 0), and Day 1, 2, 3, 4, 5, 6, 7, 8, 9, 10, 11, 12, 13, and 14.

TABLE 6
Experimental Design

| | | | Dosing | | | Duration |
|---|---|---|---|---|---|---|
| Group | Treatment | N | Level | Route | Regimen | (days) |
| 1 | Sham | 8 | 0 mg/kg | topical | Bid | 14 |
| | operation | | | | | |
| 2 | Vehicle, | 25 | 0 mg/kg, | topical | Bid | 14 |
| | 15% ethanol | | | | | |
| 3 | MCI-186 | 25 | 3 mg/kg | i.v. | q.d. | 14 |
| | (Edaravone) | | | | | |
| 4 | Acetyldiflunisal | 25 | 60 mg/kg | oral | Bid | 14 |
| 5 | Low dose | 25 | 15 mg/kg, | topical | Bid | 14 |
| 6 | Middle dose | 25 | 30 mg/kg, | topical | Bid | 14 |
| 7 | High dose | 25 | 60 mg/kg, | topical | Bid | 14 |

Before MCAO the animals were fasting overnight but allowed free access to water. Under anesthesia, the right common carotid artery (CCA), internal carotid artery (ICA), and external carotid artery (ECA) were exposed through a midline incision of the neck. A commercial monofilament (silicon-coated) was used as an occluder and inserted via the CCA. The occluder was advanced into the CCA 18±0.5 mm beyond the carotid bifurcation. Mild resistance indicated that the occluder was properly lodged in the anterior cerebral artery thus blocking the blood flow to the middle cerebral artery (MCA). After 1 hour, reperfusion was allowed by withdrawing the monofilament totally. Body temperature was kept around 36.5° C. with a heading pad during the surgery process. Clinical signs were tested at 2 hours after occlusion by the observer blinded to the treatment group to confirm neurological deficit. The neurological deficits (see Table 3) were evaluated daily afterwards until Day 14. Fourteen days after MCAO, animals were euthanized and the brains were cut into 5 coronal sections (2 mm in thickness by use of a rat brain matrix). The fresh brain sections were stained with 2% solution of triphenyltetrazolium chloride (TTC) at 37° C. and then fixed with 4% paraformaldehyde. Pictures of all sections were taken with a digital camera. These digital pictures were put into a computer. After a blind measurement of infarct area (%) for each section with Image-Pro Plus software, the infarct volumes per brain were obtained. The infarct area (%)=(contralateral hemisphere area−ipsilateral non-infarct area)/contralateral hemisphere area, then the issue swelling and atrophy were corrected with this formula.

For infarct area analysis, One-way analysis of variance (One-Way ANOVA) followed by Dunnett Multiple Comparisons Test was used for comparing the differences between treatment groups and Vehicle group. T-test was used to compare the difference between Positive control and Vehicle group. P<0.05 was considered as significant differences between the groups. Data was presented as Mean±SEM. For body weight analysis, t-test was used to compare the differences among groups in difference days after surgery.

All animals showed good general conditions before study. During this experiment mortalities were 4 of 25 (16%) in the vehicle group, 5 of 25 (20%) in the Edaravone-treated group, 3 of 25 (12%) in oral acetyldiflunisal group, 2 of 25 (8%) in low dose group of the transdermal (pyrrolidin-2-yl)methyl 2′,4′-difluoro-4-acetoxy-[1,1′-biphenyl]-3-carboxylate hydrochloride, 1 of 25 (4%) in the middle dose group and high dose group of the transdermal (pyrrolidin-2-yl)methyl 2′,4′-difluoro-4-acetoxy-[1,1′-biphenyl]-3-carboxylate hydrochloride and 0 of 8 (0%) in the sham operation group. Most of the animal death occurred between 48 hours (6 rats) and 72 hours after MCAO surgery. All other MCAO animals lost weight during Day 1 till Day 4 after surgery, and then remained in a relatively stable health condition before euthanized. Animals of sham operation group only lost weight during first 2 days after sham-surgery then gradually caught back to normal level.

Few obvious bleeding animals during surgery were abandoned. Animals were monitored carefully until consciousness was fully recovered after the last step of surgery (the occluder was removed).

The rats treated with transdermal (pyrrolidin-2-yl)methyl 2′,4′-difluoro-4-acetoxy-[1,1′-biphenyl]-3-carboxylate hydrochloride showed less infarct volumes: low dose group (37.52%±2.789, P<0.12), middle dose group (35.02%±2.827, P<0.06), high dose group (33.53%±1.986, P<0.05), when compared with i.v. Edaravone (40.35%±2.078), oral acetyldiflunisal (38.95%±2.176) and the vehicle group (41.08%±1.982), expressed as mean±SEM. The differences of high dose vs. Vehicle group were statistically significant (P<0.05). In contrast, acetyldiflunisal and MCI-186/Edaravone-treated rats demonstrated little reduction of infarct volumes. The rats in the sham group showed an unavoidable measurement variation (1.98%±0.751). Most infarction in the vehicle-, acetyldiflunisal-, and MCI-186/Edaravone-treated groups was involved in the striatum and fronto-parietal cortex that were typically supplied by the middle cerebral artery, while the infarct size in low, middle, and high dose groups were smaller with less involvement of the striatum and the corresponding cortex. The detailed analysis results of infarct volume are shown in FIG. 10.

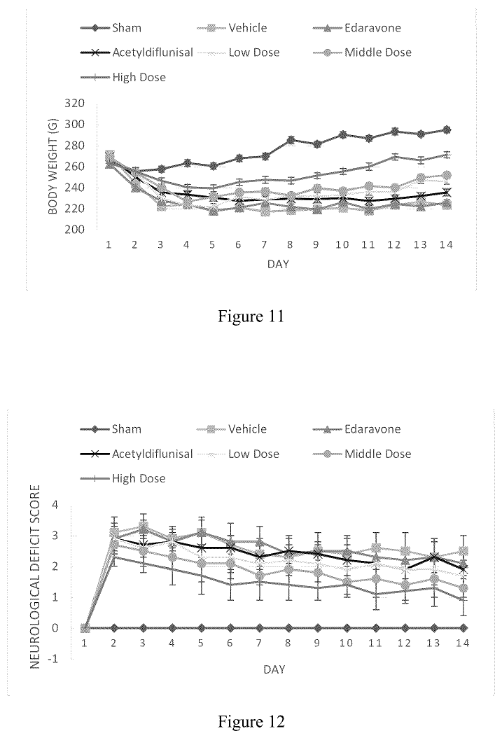

Body weight of MACO rats dropped dramatically in the first four days after surgery, and then relatively stable afterwards. While for the body weight of low (P<0.10), middle (P<0.08) and high (P<0.05) dose groups of (pyrrolidin-2-yl)methyl 2′,4′-difluoro-4-acetoxy-[1,1′-biphenyl]-3-carboxylate hydrochloride increased at Day 5-14, and showed significant difference from the vehicle group by t-test. The detailed information on assessment of body weight is shown in FIG. 11.

The ischemic insult caused the clinical signs of motor function impairments on the left side. The neurological deficit scores of high dose groups of (pyrrolidin-2-yl)methyl 2′,4′-difluoro-4-acetoxy-[1,1′-biphenyl]-3-carboxylate hydrochloride vs vehicle group show differences significantly. The detailed information on assessment of neurological deficit scores is shown in FIG. 12.

This study investigated therapeutic efficacy of (pyrrolidin-2-yl)methyl 2′,4′-difluoro-4-acetoxy-[1,1′-biphenyl]-3-carboxylate hydrochloride on cerebral ischemia in rat MCAO model. Comparing to vehicle group, high dose group of (pyrrolidin-2-yl)methyl 2′,4′-difluoro-4-acetoxy-[1,1′-biphenyl]-3-carboxylate hydrochloride exhibited good neuroprotective effect on brain ischemic injury.

### Example 18 Efficacy of (pyrrolidin-2-yl)methyl 2-(2-acetoxybenzoyl)oxybenzoate Hydrochloride in Rats with Acute Ischemia Induced by Middle Cerebral Artery Occlusion (MCAO)

The objective of this study was to investigate therapeutic efficacy of (pyrrolidin-2-yl)methyl 2-(2-acetoxybenzoyl)oxybenzoate hydrochloride for brain ischemic injury and associated neurological deficits induced by a temporal MCAO in rat.

Male SD rats (255-280 g) were used. The rats were selected for inclusion based upon acceptable clinical condition and body weight. Animals were randomly assigned to 7 groups (25 rats/group for MCAO surgery including transdermal (pyrrolidin-2-yl)methyl 2-(2-acetoxybenzoyl)oxybenzoate hydrochloride, acetylsalsalate, MCI-186/Edaravone (i.v) treatment and vehicle groups; 8 rats/group for sham surgery group). The rats were kept one week acclamation in the animal facility prior to any procedures. Animal identification number was labeled on the tail and cage tag as well. See the experimental design as illustrated in Table 7.

(Pyrrolidin-2-yl)methyl 2-(2-acetoxybenzoyl)oxybenzoate hydrochloride, 2-(2-acetoxybenzoyl)oxybenzoic acid (acetylsalsalate), Edaravone, and vehicle were administered at 1 h after MCAO surgery (Day 0), and Day 1, 2, 3, 4, 5, 6, 7, 8, 9, 10, 11, 12, 13, and 14.

TABLE 7
Experimental Design

| | | | Dosing | | | Duration |
|---|---|---|---|---|---|---|
| Group | Treatment | N | Level | Route | Regimen | (days) |
| 1 | Sham | 8 | 0 mg/kg | topical | Bid | 14 |
| | operation | | | | | |
| 2 | Vehicle, | 25 | 0 mg/kg, | topical | Bid | 14 |
| | 15% ethanol | | | | | |
| 3 | MCI-186 | 25 | 3 mg/kg | i.v. | q.d. | 14 |
| | (Edaravone) | | | | | |
| 4 | Acetylsalsalate | 25 | 60 mg/kg | oral | Bid | 14 |
| 5 | Low dose | 25 | 15 mg/kg, | topical | Bid | 14 |
| 6 | Middle dose | 25 | 30 mg/kg, | topical | Bid | 14 |
| 7 | High dose | 25 | 60 mg/kg, | topical | Bid | 14 |

Before MCAO the animals were fasting overnight but allowed free access to water. Under anesthesia, the right common carotid artery (CCA), internal carotid artery (ICA), and external carotid artery (ECA) were exposed through a midline incision of the neck. A commercial monofilament (silicon-coated) was used as an occluder and inserted via the CCA. The occluder was advanced into the CCA 18±0.5 mm beyond the carotid bifurcation. Mild resistance indicated that the occluder was properly lodged in the anterior cerebral artery thus blocking the blood flow to the middle cerebral artery (MCA). After 1 hour, reperfusion was allowed by withdrawing the monofilament totally. Body temperature was kept around 36.5° C. with a heading pad during the surgery process. Clinical signs were tested at 2 hours after occlusion by the observer blinded to the treatment group to confirm neurological deficit. The neurological deficits (see Table 3) were evaluated daily afterwards until Day 14. Fourteen days after MCAO, animals were euthanized and the brains were cut into 5 coronal sections (2 mm in thickness by use of a rat brain matrix). The fresh brain sections were stained with 2% solution of triphenyltetrazolium chloride (TTC) at 37° C. and then fixed with 4% paraformaldehyde. Pictures of all sections were taken with a digital camera. These digital pictures were put into a computer. After a blind measurement of infarct area (%) for each section with Image-Pro Plus software, the infarct volumes per brain were obtained. The infarct area (%)=(contralateral hemisphere area−ipsilateral non-infarct area)/contralateral hemisphere area, then the issue swelling and atrophy were corrected with this formula.

For infarct area analysis, One-way analysis of variance (One-Way ANOVA) followed by Dunnett Multiple Comparisons Test was used for comparing the differences between treatment groups and Vehicle group. T-test was used to compare the difference between Positive control and Vehicle group. P<0.05 was considered as significant differences between the groups. Data was presented as Mean±SEM. For body weight analysis, t-test was used to compare the differences among groups in difference days after surgery.

All animals showed good general conditions before study. During this experiment mortalities were 5 of 25 (20%) in the vehicle group, 5 of 25 (20%) in the Edaravone-treated group, 3 of 25 (12%) in oral acetylsalsalate group, 2 of 25 (8%) in low dose group of the transdermal (pyrrolidin-2-yl)methyl 2-(2-acetoxybenzoyl)oxybenzoate hydrochloride, 1 of 25 (4%) in the middle dose group of transdermal (pyrrolidin-2-yl)methyl 2-(2-acetoxybenzoyl)oxybenzoate hydrochloride and 0 of 25 in high dose group of the transdermal (pyrrolidin-2-yl)methyl 2-(2-acetoxybenzoyl)oxybenzoate hydrochloride and 0 of 8 (0%) in the sham operation group. Most of the animal death occurred between 48 hours (6 rats) and 72 hours after MCAO surgery. All other MCAO animals lost weight during Day 1 till Day 4 after surgery, and then remained in a relatively stable health condition before euthanized. Animals of sham operation group only lost weight during first 2 days after sham-surgery then gradually caught back to normal level.

Few obvious bleeding animals during surgery were abandoned. Animals were monitored carefully until consciousness was fully recovered after the last step of surgery (the occluder was removed).

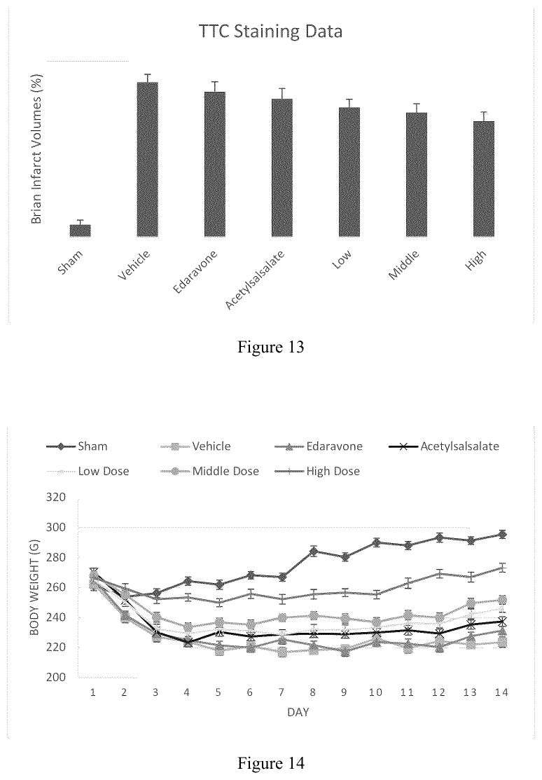

The rats treated with transdermal (pyrrolidin-2-yl)methyl 2-(2-acetoxybenzoyl)oxybenzoate hydrochloride showed less infarct volumes: low dose group (36.87%±2.265, P<0.10), middle dose group (35.39%±2.479, P<0.07), high dose group (32.98%±2.546, P<0.05), when compared with i.v. Edaravone (41.33%±2.792), oral acetylsalsalate (39.25%±2.981) and the vehicle group (43.98%±2.256), expressed as mean±SEM. The differences of high dose vs. Vehicle group were statistically significant (P<0.05). In contrast, acetylsalsalate and MCI-186/Edaravone-treated rats demonstrated little reduction of infarct volumes. The rats in the sham group showed an unavoidable measurement variation (3.56%±1.257). Most infarction in the vehicle-, acetylsalsalate-, and MCI-186/Edaravone-treated groups was involved in the striatum and fronto-parietal cortex that were typically supplied by the middle cerebral artery, while the infarct size in low, middle, and high dose groups were smaller with less involvement of the striatum and the corresponding cortex. The detailed analysis results of infarct volume are shown in FIG. 13.

Body weight of MACO rats dropped dramatically in the first four days after surgery, and then relatively stable afterwards. While for the body weight of low (P<0.10), middle (P<0.08) and high (P<0.05) dose groups of (pyrrolidin-2-yl)methyl 2-(2-acetoxybenzoyl)oxybenzoate hydrochloride increased at Day 5-14, and showed significant difference from the vehicle group by t-test. The detailed information on assessment of body weight is shown in FIG. 14.

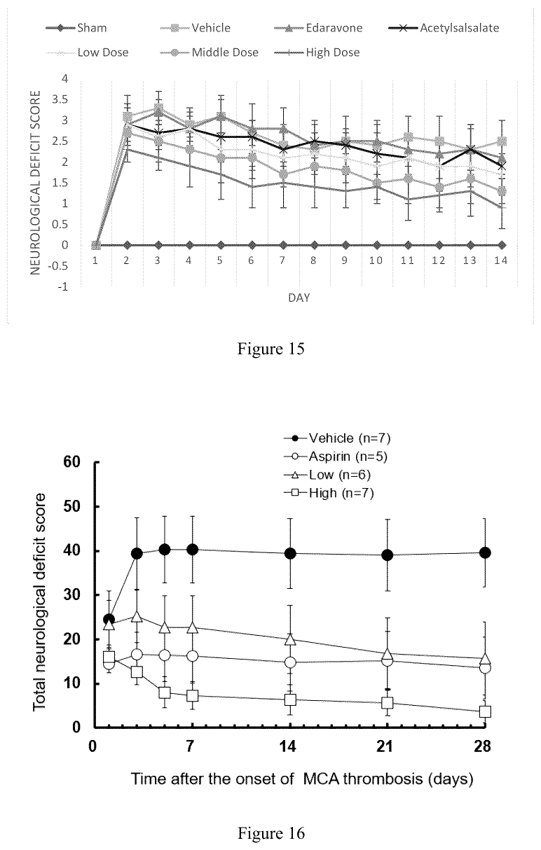

The ischemic insult caused the clinical signs of motor function impairments on the left side. The neurological deficit scores of high dose groups of (pyrrolidin-2-yl)methyl 2-(2-acetoxybenzoyl)oxybenzoate hydrochloride vs vehicle group show differences significantly. The detailed information on assessment of neurological deficit scores is shown in FIG. 15.

This study investigated therapeutic efficacy of (pyrrolidin-2-yl)methyl 2-(2-acetoxybenzoyl)oxybenzoate hydrochloride on cerebral ischemia in rat MCAO model. Comparing to vehicle group, high dose group of (pyrrolidin-2-yl)methyl 2-(2-acetoxybenzoyl)oxybenzoate hydrochloride exhibited good neuroprotective effect on brain ischemic injury.

### Example 19 Effects of 2-(diethylamino)ethyl Acetoxybenzoate Hydrochloride on Neurological Deficits and Cerebral Infarcts in a Middle Cerebral Artery (MCA) Thrombosis Model of Monkeys

This study was conducted to investigate whether 2-(diethylamino)ethyl acetoxybenzoate hydrochloride would improve neurological deficits and cerebral infarcts in a middle cerebral artery (MCA) thrombosis model of monkeys. The studies in Examples 14-21 are very different from other published studies. In the studies of Examples 14-21, the treatment with the drugs were started from 1 hour to 60 days post thrombosis (i.e., the formation of a blood clot in a blood vessel), strokes and heart attacks, which means that the treatment is for functional recovery or improvement post strokes, heart attacks, heart failure or other cardiovascular diseases. In contrast, in other published studies, the treatment with drugs were started before thrombosis, which means that the treatment with drugs is for prevention, not for recovery or improvement from the damages of thrombosis.

Formulations were prepared for every week and stored at 2-8° C. in amber bottles when not in use and used within 7 days. Concentrations were expressed as free base of the test article. A correction factor of 1.13 and purity were used to calculate the free base.

2-(Diethylamino)ethyl acetoxybenzoate hydrochloride (15.82 g) was weighed out and added to 200 ml volumed flask. About 80% of final volume of 15% (v/v) ethanol in sterile water for injection (Vehicle) was added to the container, stirred the resulting mixture until a clear solution was obtained, then more 15% (v/v) ethanol was added into the flask until final volume of 200 ml. (70 mg/ml of 2-(diethylamino)ethyl acetoxybenzoate free base.)

The administration of 2-(diethylamino)ethyl acetoxybenzoate hydrochloride, aspirin or vehicle was started at 3 hr after the onset of rose bengal injection. From the next day until Day 27, 2-(diethylamino)ethyl acetoxybenzoate hydrochloride, aspirin or vehicle were administered twice per day. 2-(Diethylamino)ethyl acetoxybenzoate hydrochloride and vehicle were administered transdermally to the back using a disposal syringe. Aspirin was administered orally by gavage using a nasal catheter with a disposal syringe. The catheter was inserted into the stomach through the mouth. The treatment was once per day of MCA thrombosis (day 0), twice per day for 27 days. (See the experimental design as illustrated in Table 8)

Monkeys were fed twice everyday about 50-100 g diet per monkey every time with fruit or vegetables which was washed and disinfected followed. Tap water was provided ad libitum during the study periods. Fluorescent lighting provided illumination approximately 12 hours per day.

Certified monkey diet (Beijing Keaoxieli Feed Co., Ltd) was provided. Nutritional ingredient of each batch of the diet was analyzed. The diet should meet the feed standards according to the State Standard of the People's Republic of China GB14924.3-2010 and GB14924.2-2001. The copy of the analytic results was retained in the study files.

Qualified tap water was used. The appearance and microorganism of drinking water were analyzed monthly and the toxicology index (such as lead and mercury) was detected annually. The water shall meet the drinking water standards according to the State Standard of the People's Republic of China GB5749-2006. The copy of the analytic results was retained in the study files.

TABLE 8
Composition of experimental groups

| Group | Administration | n |
|---|---|---|
| Vehicle | 0.430 mL/kg/time, twice a day | 6 |
| Aspirin | 20 mg/4.0 mL/kg/time, twice a day | 6 |
| Low Dose of Test Article | 15 mg/0.215 mL/kg/time, twice a day | 6 |
| High Dose of Test Article | 30 mg/0.430 mL/kg/time, twice a day | 6 |

The experimental groups were assigned according to the minimization method based on the preoperative body weights of 24 monkeys.

The information about assignment was disclosed to only the person in charge of administration but not those responsible for preparation of a MCA thrombosis model, assessment of neurological deficits, and measurement of infarct lesions

The preparation of the MCA thrombosis model was carried out in a blind manner. The operators were not those responsible for grouping and administration.

The monkeys that had been fastened for at least 12 h were anesthetized with intramuscular administration of ketamine hydrochloride (20 mg/kg), and placed on an operation-bed. Anesthesia was continued with 1.0% isoflurane in O₂ gas until 30 min. The inhalation anesthesia was performed with anesthesia machines (SN23402, Hallowell engineering and manufacturing corporation, USA) and respirators (SN23402, Hallowell engineering and manufacturing corporation, USA), and placed on an operation-bed. During anesthesia, body temperature of the animals (rectal temperature) was controlled between 37.0±0.5° C. by heating mattresses (XMTA-7000, Delixi Electric., Ltd.).

Under anesthesia, the left eye was enucleated using a bipolar coagulation device (Valleylab Force 1C, USA). A craniotomy was then performed supralaterally to the left side of the optic nerve. The next day animals were anesthetized with ketamine hydrochloride (20 mg/kg) and the dura and subarachnoid membrane were opened. MCA thrombosis was induced by photochemical reaction. The proximal part of the main MCA trunk. was irradiated with green light wave length 532 nm achieved by use of a xenon lamp (GL532T3-100FC, Shanghai Laser& Optics Century Col., Ltd.). The irradiation was directed by a 3-mm diameteroptic fiber mounted on a micromanipulator. Irradiation was started and rose bengal 20 mg/kg was injected intravenously. Photoirradiation was continued for a 30 min. The dura was then covered with a moist gelatin sponge.

Penicillin G Potassium (100,000 U/animal) was intramuscularly injected daily for 3 days after surgery in order to prevent postoperative infection.

Neurological deficits (see Table 3) were assessed 1, 3, 5, 7, 14 and 28 days after the onset of MCA thrombosis (day 0) in a blind manner. The neurological deficits was scored on consciousness, sensory system, motor system and skeletal muscle coordination, and the total neurological deficit score was calculated by summing the scores of consciousness, sensory system, motor system and skeletal muscle coordination. The neurological deficit score was measured by adapting the scoring method described in Kito G, et al. J Neurosci Meth, 2001, 105:45-53).

The survival monkeys were deeply anesthetized with pentobarbital sodium (35 mg/kg, i.v.) 28 days after the onset of MCA thrombosis. After cutting the atriums of heart, 200 mL of heparinized physiological saline (10 U/mL) was allowed to perfuse the brain via the common carotid arteries, and followed by perfusion of 200 mL of 10% formalin neutral buffer solution. The whole brain was removed, and then was put in pathology sample preservation packs containing 10% formalin neutral buffer solution. For each brain sample, the coronal sections (thickness: 7-8 μm) per site were prepared at 10 sites at 4-mm intervals. Slices per site were each stained with H & E reagent.

Measurement of cerebral infarct lesions was carried out in a blind manner. The infarct lesions in the ischemic side are defined as a necrotic portion compared with that in the contralateral side. The infarcted area of left cerebral hemisphere (A) in the cross-sections was traced and measured using a computerized image analysis system (image J).

When the infarct size of 2 serial section was made into D and D′, the infarcted volume using the thickness (4 mm) of a section is measured in the following formula:

$$Infarcted volume = \{ ( D + {D}^{′} ) \times 4 \} / 2$$

Total infarcted volume is calculated by summing the infarcted volume of the 10 serial sections.

### Evaluation Items:

1) Neurological deficit scores (consciousness, sensory system, motor system and skeletal muscle coordination) and the total neurological deficit score
2) Infarct lesion volumes

### Results:

Mortality rate of vehicle, aspirin, low dose group of 2-(diethylamino)ethyl acetoxybenzoate hydrochloride and high dose group of 2-(diethylamino)ethyl acetoxybenzoate hydrochloride were 4/7, 1/5, 0/5 and 0/7 respectively. Therefore, neurological deficits after death of animals were expressed with the score at the time of death. Severe neurological deficits were observed in vehicle group at three days after MCA thrombosis. These neurological deficits did not show spontaneous recovery. The neurological deficits in Aspirin group tended to show slight recovery until 28 days after MCA thrombosis. low dose group of 2-(diethylamino)ethyl acetoxybenzoate hydrochloride and high dose group of 2-(diethylamino)ethyl acetoxybenzoate hydrochloride dose-dependently improved the neurologic deficits. (See FIG. 16)

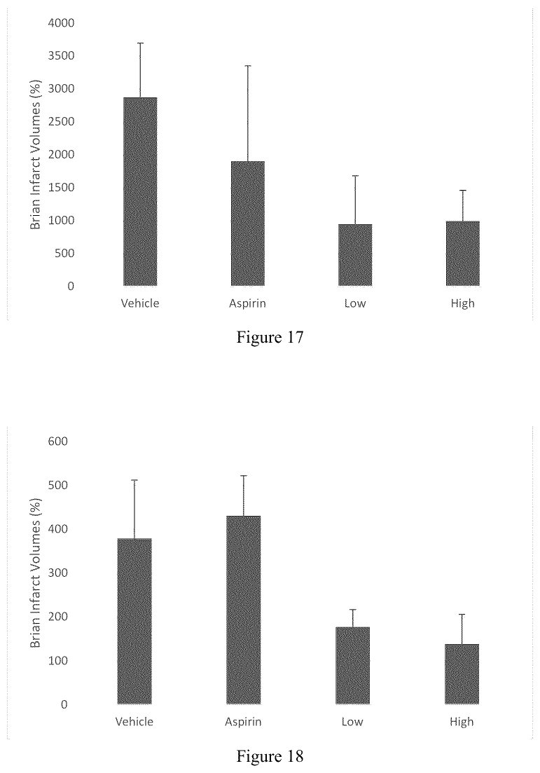

The infarct volume measured at Day 28 after MCA thrombosis showed 2873, 1901, 945 and 988 mm³ in vehicle, aspirin, low dose group of 2-(diethylamino)ethyl acetoxybenzoate hydrochloride and high dose group of 2-(diethylamino)ethyl acetoxybenzoate hydrochloride, respectively (see FIG. 17). The low dose group of 2-(diethylamino)ethyl acetoxybenzoate hydrochloride and high dose group of 2-(diethylamino)ethyl acetoxybenzoate hydrochloride reduced the infarcted volume significantly.

In the infarct area, severe liquefaction was observed in vehicle group, but Aspirin group and low dose group of 2-(diethylamino)ethyl acetoxybenzoate hydrochloride were moderate, and high dose group of 2-(diethylamino)ethyl acetoxybenzoate hydrochloride was slight. In the penumbra area, many gemistocytic astrocytes were observed in high dose group of 2-(diethylamino)ethyl acetoxybenzoate hydrochloride, but only few gemistocytic astrocytes were observed in vehicle group. Aspirin group showed hemorrhage in the penumbra area. In the distant area, delayed neuronal death was observed in all groups, but 2-(diethylamino)ethyl acetoxybenzoate hydrochloride showed slightly.

2-(Diethylamino)ethyl acetoxybenzoate hydrochloride reduced the mortality rate and the infarct volume, and ameliorated the neurologic deficits. In the histopathological findings, many gemistocytic astrocytes were observed in high group of 2-(diethylamino)ethyl acetoxybenzoate hydrochloride. Since liquefaction and delayed neuronal death were slight in 2-(diethylamino)ethyl acetoxybenzoate hydrochloride, many gemistocytic astrocytes remained. In conclusion, we demonstrated that 2-(diethylamino)ethyl acetoxybenzoate hydrochloride ameliorated ischemic brain damage and delayed neuronal death in a middle cerebral artery thrombosis model of monkeys.

### Example 20 Effects of 2-(diethylamino)ethyl Acetoxybenzoate Hydrochloride on Myocardial Infarction Efficacy Study in Minipigs

### Preparation of Formulation

2-(Diethylamino)ethyl acetoxybenzoate hydrochloride was dissolved in 15% ethanol to the specified concentrations (7.91%) for transdermal administration and Aspirin was dissolved in 0.5% MC to the specified concentrations (0.5%) for oral administration.

2-(Diethylamino)ethyl acetoxybenzoate hydrochloride and vehicle were administered transdermally to the back using a disposal syringe, twice a day from the next day until Day 13, Aspirin was administered orally by gavage using a nasal catheter with a disposal syringe. The catheter was inserted into the stomach through the mouth, twice a day from the next day until Day 13.

Based on pharmacological effect levels, the dose levels were selected as the following:

2-(diethylamino)ethyl acetoxybenzoate hydrochloride: low dose: 15 mg/0.215 mL/kg/time (in free base, equal to 10 mg/kg/tome aspirin), twice a day; high dose: 30 mg/0.430 mL/kg/time (in free base, equal to 20 mg/kg/tome aspirin), twice a day; aspirin: 20 mg/4 mL/kg/time, twice a day.

Dose formulations were prepared for every week and stored at 2-8° C. to in amber bottles when not in use and used within 7 days. Concentrations were expressed as free base of the test article. A correction factor of 1.13 and purity were used to calculate the free base.

2-(Diethylamino)ethyl acetoxybenzoate hydrochloride (15.82 g) was weighed out and added to a 200 ml volumed flask. About 80% of final volume of 15% (v/v) ethanol in sterile water for injection (Vehicle) was added to the container, stirred the resulting mixture until a clear solution was obtained, then more 15% (v/v) ethanol was added into the flask until final volume of 200 ml (70 mg/ml of 2-(diethylamino)ethyl acetoxybenzoate free base).

Total 24 male minipigs were used for this study. The minipigs were about 2-3 weeks of age when this study was started. The minipigs were already quarantined and acclimated. Before animal being transferred from holding colony, a physical examination was performed and the acceptability of the animals for the study was evaluated by veterinary. See the experimental design as illustrated in Table 9.

TABLE 9
Composition of experimental groups

| Group | Administration | n |
|---|---|---|
| Vehicle | 0.430 mL/kg/time, twice a day | 6 |
| Aspirin | 20 mg/4.0 mL/kg/time, twice a day | 6 |
| Low Dose of Test Article | 15 mg/0.215 mL/kg/time, twice a day | 6 |
| High Dose of Test Article | 30 mg/0.430 mL/kg/time, twice a day | 6 |

The experimental groups were assigned according to the minimization method based on the preoperative body weights of 24 minipigs.

The preparation of the myocardial in model was carried out in a blind manner. The operators were not those responsible for grouping and administration.

The minipigs that had been fastened for at least 12 h were anesthetized with intramuscular administration of pentobarbital (25-50 mg/kg), and placed on an operation-bed. Anesthesia was continued with 1.0% isoflurane in O₂ gas. The inhalation anesthesia was performed with anesthesia machines (SN23402, Hallowell engineering and manufacturing corporation, USA) and respirators (SN23402, Hallowell engineering and manufacturing corporation, USA), and placed on an operation-bed. During anesthesia, body temperature of the animals (rectal temperature) was controlled between 37.0±0.5° C. by heating mattresses (XMTA-7000, Delixi Electric., Ltd.).

Under anesthesia, thoracotomy was carried out and the circumflex coronary artery of the heart was exposed. The circumflex coronary artery thrombosis was induced by photochemical reaction. The artery was irradiated with green light wavelength 532 nm achieved by use of a xenon lamp (GL532T3-100FC, Shanghai Laser& Optics Century Co., Ltd.). The irradiation was directed by a 3-mm diameteroptic fiber mounted on a micromanipulator. Irradiation was started and rose bengal 20 mg/kg was injected intravenously. Photoirradiation was continued for a 30 min.

Penicillin G Potassium (100,000 U/animal) was intramuscularly injected daily for 3 days after surgery in order to prevent postoperative infection.

Before the preparation of the myocardial infarction model, the telemetry transmitter (TL11M2-D70-PCT, Data Sciences International, MN, USA) was put into the jacket, and the ECG leads were subcutaneously placed on the right lateral thorax and the left lateral abdomen. Physiological signals (ECG) were recorded within 24 hours after rose bengal administration.

The survival minipigs were deeply anesthetized with pentobarbital sodium (30 mg/kg, i.v.) 14 days after the onset of the circumflex coronary artery thrombosis. After cutting the femoral artery, thoracotomy was carried out and the heart was removed. The heart was cut with 5-mm width in five blocks, and then these blocks were immersed in 37° C. TTC regent for 5 min. After staining with TTC reagent, the photograph of the heart 5 blocks was taken. All blocks were immersed in 10% formalin neutral buffer solution. The 2nd block appeared large necrotic area was cut sections (thickness: 7-8 μm). Slice was stained with H & E reagent.

Measurement of myocardial infarct lesions was carried out in a blind manner.

The infarct lesions were defined as a necrotic portion of TTC stained blocks. When the infarct size of 2 serial section was made into D and D′, the infarcted volume using the thickness (5 mm) of a block was measured in the following formula:

$$Infarcted volume = \{ ( D + {D}^{′} ) \times 5 \} / 2$$

Total infarcted volume was calculated by summing the infarcted volume of the 5 serial sections.

The histopathological alteration (necrosis, inflammation, granulation, etc.) of myocardial infarction was examined using H & E stained slides.

### Evaluation Items

1) Infarct lesion volumes
2) histopathological alteration

### Results:

The infarct volume measured 28 days after MCA thrombosis showed 379, 431, 177 and 138 mm³ in vehicle, aspirin, low dose group of 2-(diethylamino)ethyl acetoxybenzoate hydrochloride were moderate, and high dose group of 2-(diethylamino)ethyl acetoxybenzoate hydrochloride, respectively. low dose group of 2-(diethylamino)ethyl acetoxybenzoate hydrochloride were moderate, and high dose group of 2-(diethylamino)ethyl acetoxybenzoate hydrochloride reduced the infarcted volume. (See FIG. 18)

In vehicle and aspirin group, moderate degeneration of myocardium and myocardial necrosis were observed. However, these histopathological changes were slight in low dose group of 2-(diethylamino)ethyl acetoxybenzoate hydrochloride were moderate and high dose group of 2-(diethylamino)ethyl acetoxybenzoate hydrochloride.

The objective of this study was to evaluate effect of 2-(diethylamino)ethyl acetoxybenzoate hydrochloride on the myocardial infarction in minipigs. In ECG assessment, increase of ST and abnormal Q wave in all group was noted, so we recognized onset of myocardial ischemia after irradiation. At Day 28 after MCA thrombosis, the myocardial infarct volume decreased in 2-(diethylamino)ethyl acetoxybenzoate hydrochloride groups. Therefore, this experiment has demonstrated that myocardial infarction was ameliorated by repeated administration of 2-(diethylamino)ethyl acetoxybenzoate hydrochloride.

### Example 20 Nonclinical Pharmacology and Toxicology

A battery of pharmacology and toxicology studies were conducted to support early clinical trials, including a GLP acute dermal maximum tolerated dose study in rats, a GLP acute dermal maximum tolerated dose study in minipigs, a 28-day GLP repeated dermal dose toxicity and toxicokinetics study in rats with 14-day recovery, a 28-day GLP repeated dermal dose toxicity and toxicokinetics study in minipigs with 14-day recovery, a GLP bacterial reverse mutation assay (Ames), a GLP in vitro chromosome aberration assay in CHO-WBL Cells, a GLP in vivo bone marrow micronucleus assay in rats, a behavioral effects in rats using the functional observational battery, a rat respiratory safety pharmacology study, a cardiovascular telemetry study in the unrestrained conscious minipigs, a skin irritation study in rabbits, and a sensitization test in guinea pigs.

From the study results, 2-(diethylamino)ethyl acetoxybenzoate hydrochloride appears to be safe and generally well tolerated.

### Example 21 Phase 1 Clinical Study

A randomized, double-blind, placebo-controlled, dose-escalation study was conducted to evaluate the safety, tolerability and PK of single and multiple doses of 2-(diethylamino)ethyl acetoxybenzoate hydrochloride following escalating single and multiple doses administered as a topical spray application.

In each of 4 dose escalation cohorts, 8 subjects were randomized to receive active study drug and 2 were randomized to matching placebo. The dose levels studied are shown in Table 10.

TABLE 10
Total Dose and Number of Sprays per Dose by Cohort

| Cohort | Total Dose | Number of Sprays per Dose |
|---|---|---|
| 1 | 70 mg | 10 sprays |
| 2 | 175 mg | 25 sprays |
| 3 | 350 mg | 50 sprays |
| 4 | 700 mg | 100 sprays |

Each cohort began with single dose administration of study drug on Day 1 of the cohort's study schedule. Serial blood samples for plasma PK analysis of 2-(diethylamino)ethyl acetoxybenzoate hydrochloride and three 2-(diethylamino)ethyl acetoxybenzoate hydrochloride metabolites, specifically, 2-(diethylamino)ethyl 2-hydroxybenzoate hydrochloride, aspirin (acetylsalicylic acid, ASA) and salicylic acid (SA) were collected over the 120-hour post-dose period.

Following a 5-day washout, multiple doses of 2-(diethylamino)ethyl acetoxybenzoate hydrochloride were administered b.i.d. for 7 days (Days 6-12, with only a morning dose administered on Day 12). Blood samples for PK analysis were collected for 120 hours post-dose period following the final dose of 2-(diethylamino)ethyl acetoxybenzoate hydrochloride administered on the morning of Day 12.

A review of safety data was performed prior to initiation of multiple dosing within a cohort and prior to dose escalation to a higher single dose. In the absence of dose-limiting adverse events (AEs) and laboratory toxicities, the next higher-dose cohort was initiated. Dose escalation and multiple dosing decisions were based on safety and tolerability assessments and agreed upon by Techfields Inc., the Medical Monitor and the Principal Investigator (PI).

The maximum individual dose of 2-(diethylamino)ethyl acetoxybenzoate hydrochloride was 700 mg b.i.d. (Cohort 4) and the maximum daily dose was 1400 mg.

For single dose PK analysis, serial blood samples were collected following the Day 1 dose at the following time points: 0 (pre-dose), 0.25, 0.5, 0.75, 1, 1.5, 2, 2.5, 3, 4, 5, 6, 8, 10, 12, 18, 24, 48, 72, 96 and 120 hours post-dose. For multiple-dose PK analysis, serial blood samples were collected following the last morning dose on Day 12 according to the same schedule used for the single-dose PK analysis. In addition, daily trough PK samples were collected prior to the morning dose on Days 7 through 11.

Safety assessments included monitoring of AEs, vital signs (blood pressure [BP], pulse rate, respiratory rate and oral temperature), clinical laboratory findings, 12-lead electrocardiograms (ECGs), skin irritation assessments and physical examination (PE) findings.

A physical examination and resting 12-lead ECG were performed at screening and on Day 13. Vitals signs were assessed at screening, at admission to the study site on Day-1 and Day 5 and prior to and 1 hour following each dose administration.

Clinical laboratory tests (chemistry, hematology and urinalysis) were performed at screening, admission on Day-1 and on Day 4, Day 13 and Day 17. Stool guaiac testing was performed at screening and at the End-of-Study visit on Day 17. Skin irritation assessments were performed prior to and 30 minutes following each dose administration.

### Conclusions:

### Safety Conclusions:

1. The study demonstrated that 2-(diethylamino)ethyl acetoxybenzoate hydrochloride was safe and well tolerated at all dose regimens studied: single doses of up to 700 mg and multiple doses up to 700 mg b.i.d. There was not an apparent relationship between dose and incidence of TEAEs
2. No subject had a serious adverse event (SAE) or a TEAE leading to discontinuation from the study.
3. Other than the TEAEs noted there were/were not overall clinically meaningful or significant changes in clinical safety parameters or physical examination findings.

### PK Conclusions:

1. Following a single dose or multiple dose administrations of 70 mg to 700 mg of 2-(diethylamino)ethyl acetoxybenzoate hydrochloride plasma concentrations of TF0039 and ASA were below quantifiable limit.
2. Following a single dose or multiple dose administrations of 70 mg to 700 mg of 2-(diethylamino)ethyl acetoxybenzoate hydrochloride, the maximum plasma concentrations of 2-(diethylamino)ethyl acetoxybenzoate hydrochloride was near the low limit of quantifiable level.
3. Following a single dose administration of 70 mg to 700 mg of 2-(diethylamino)ethyl acetoxybenzoate hydrochloride, salicylic acid was the predominant entity in the plasma; both AUCt and AUCτ of salicylic acid increased with 2-(diethylamino)ethyl acetoxybenzoate hydrochloride dose. Cₘₐₓ of salicylic acid increased from 2-(diethylamino)ethyl acetoxybenzoate hydrochloride dose of 350 mg to 700 mg. Following multiple dose administrations of 2-(diethylamino)ethyl acetoxybenzoate hydrochloride from 70 mg to 700 mg, Tₘₐₓ of salicylic acid was significantly different from the values derived from the single dose treatment. Under proportional increase in Cₘₐₓ and AUC_{τ} of SA with dose was observed. Terminal half-life of salicylic acid following either a single dose or multiple doses of 2-(diethylamino)ethyl acetoxybenzoate hydrochloride was in the range of 23 to 36 hr.
4. The accumulation of salicylic acid (R_Cₘₐₓ and R_AUCτ) at steady state ranged from 1.5 to 2.6 for Cₘₐₓ, and 2 to 8 for AUCT.
5. Large variability in PK parameters for both 2-(diethylamino)ethyl acetoxybenzoate hydrochloride and salicylic acid was observed.
6. 2-(Diethylamino)ethyl acetoxybenzoate hydrochloride is readily and almost completely converted (≥99%) to salicylic acid following 2-(diethylamino)ethyl acetoxybenzoate hydrochloride topical spray.

### Example 22 Phase 2 Clinical Study

The phase 2 clinical trial was a multicenter, randomized, double-blind (within dose), placebo-controlled, parallel-group, dose-range-finding study to evaluate the efficacy and safety of 2-(diethylamino)ethyl acetoxybenzoate hydrochloride spray versus placebo in functional improvement of patients with ischemic strokes, measured by the dichotomized mRS score (0 to 2 versus >2) for all randomized patients at Week 16. In addition, the study was also to assess the safety and tolerability of multiple doses of 2-(diethylamino)ethyl acetoxybenzoate hydrochloride spray at Week 16 and Week 32, the efficacy of 2-(diethylamino)ethyl acetoxybenzoate hydrochloride spray compared with placebo for functional improvement of patients with ischemic strokes measured by the dichotomized mRS score (0 to 2 versus >2) for all randomized patients at Week 32, and the effect of 2-(diethylamino)ethyl acetoxybenzoate hydrochloride spray compared with that of placebo for functional improvement of patients with ischemic strokes measured by improvement of the National Institute of Health Stroke Scale (NIHSS) score or the Barthel Index (BI) from baseline to Week 16 and Week 32,

### Methodology/Study Design

Each patient enrolled in Group A was administered the investigational drug in a double-blind manner for 16 weeks starting between 3 and 60 days after the onset of stroke symptoms (Day 1 of the study). The patients and/or their caregivers were instructed how to apply the investigational drug accurately so that patients or their caregivers would administer 20 sprays/dose of either the investigational drug or placebo twice daily (approximately every 12 hours) for a total daily dose of 280 mg of investigational drug. The 20 sprays of investigational drug were administered as follows: 4 sprays around the neck, 2 sprays to the left shoulder, 2 sprays to the right shoulder, 6 sprays to the chest, 3 sprays to the left leg, and 3 sprays to the right leg. Each spray was sprayed on a different area of the skin to reach maximum absorption. The patient would wait for at least 5 minutes to dress after the last spray so that the skin became completely dry.

Patients were asked to return to the study site at Weeks 4, 8, 12, and 16 for efficacy and safety assessments. After the Week 16 assessments had been performed, patients would start the open-label period of the study the next day and receive active treatment in an open-label manner through the end of the study (EOS) at Week 32. That is, patients who were receiving active treatment would continue to receive active treatment, and patients who were receiving placebo would switch to active treatment for the remaining 16 weeks of the study. Patients would receive only 1 dose of investigational drug on the Week 16 visit day. Patients would return to the study site at Week 24 and Week 32 (EOS) for efficacy and safety assessments. An Early Termination visit would occur if the patient was discontinued from the study prematurely. Patients would have a follow-up visit approximately 14 days after the Early Termination visit or the Week 32 (EOS) visit.

### Test Product, Dose, Dosage Form, and Mode of Administration:

The test product was a 7.91% solution of 2-(diethylamino)ethyl acetoxybenzoate hydrochloride (7% of solution of 2-(diethylamino)ethyl acetoxybenzoate) or placebo in 15% ethanol, which was administered transdermally as a spray. The 7.91% transdermally spray solution consists of 1582 mg 2-(diethylamino)ethyl acetoxybenzoate hydrochloride (equivalent to 1400 mg 2-(diethylamino)ethyl acetoxybenzoate free base) in 20 mL 15% ethanol (v/v). The spray bottle deposits 100 ml of spray solution and 7 mg 2-(diethylamino)ethyl acetoxybenzoate free base per spray on the skin. Subjects were to apply each spray to a different skin area around the neck, chest, legs and arms based on the randomized dose level assigned.

### Dose and Regimen:

Patients were randomly assigned in 1:1:1 ratio to Group A, Group B, or Group C and further randomized within each treatment group in a 2:1 ratio to receive either 2-(diethylamino)ethyl acetoxybenzoate hydrochloride 140 mg/dose (20 sprays/dose) or placebo in Group A, 70 mg/dose (10 sprays/dose) or placebo in Group B or 35 mg/dose (5 sprays/dose) or placebo in Group C.

The treatment was administered twice daily (approximately every 12 hours) starting on Day 1 of the study through the Week 16 visit. Each spray was sprayed on a different area of the skin to reach maximum absorption.

At the Week 16 visit, the first dose was administer at the clinical site. Starting the day after the Week 16 visit, the active treatment was administered in an open-label manner through the end of the study (Week 32). That is, patients

Group A administered the 20 sprays of investigational drug as follows: 4 sprays around the neck, 2 sprays to the left shoulder, 2 sprays to the right shoulder, 6 sprays to the chest, 3 sprays to the left leg, and 3 sprays to the right leg twice daily for a total of 40 sprays (280 mg)/day. Each spray was sprayed on a different area of the skin to reach maximum absorption.

Group B administered the investigational drug as follows: 2 sprays to the front of the neck, 1 spray to the left side of the neck, 1 spray to the right side of the neck, 6 sprays to the chest close to the neck twice daily for a total of 20 sprays (140 mg)/day. Each spray was sprayed on a different area of the skin to reach maximum absorption.

Group C administered the investigational drug as follows: 1 spray to the front of the neck, 1 spray to the left side of the neck, 1 spray to the right side of the neck, 2 sprays to the chest twice daily for a total of 10 sprays (70 mg/day). Each spray was sprayed on a different area of the skin to reach maximum absorption.

### Efficacy Evaluation

### The Primary Efficacy Endpoint

The primary efficacy endpoint is the mRS score at Week 16 dichotomized as ‘success’ (0 to 2) or failure (>2) for all randomized patients at Week 16.

### Modified Ranking Scale Score

The mRS score was assessed at screening; Day 1 (baseline); Weeks 4, 8, 12, 16, 24, and 32; Early Termination Visit (if applicable) and Follow-Up Visit.

The mRS score measures the patient's functional level of activity and is dichotomized as a favorable outcome (score=0-2) versus unfavorable (score ≥2). The mRS score ranges from 0 (no symptoms) to 6 (death) as follows:
0=No symptoms at all
1=No significant disability despite symptoms; able to carry out all usual duties and activities
2=Slight disability; unable to carry out all previous activities, but able to look after own affairs without assistance
3=Moderate disability requiring some help, but able to walk unassisted
4=Moderate-severe disability; unable to walk without assistance and unable to attend to own bodily needs without assistance.
5=Severe disability; bedridden, incontinent, and requiring constant nursing care and attention.
6=Dead.

### Secondary Endpoints

Secondary efficacy endpoints include the change from baseline in NIHSS score, BI score, and GOS-E score for all randomized patients at Week 16 and Week 32, and in mRS score at Week 32. The NIHSS, and BI scores are assessed at screening; Day 1 (baseline); Weeks 4, 8, 12, 16, 24, and 32. Another secondary endpoint is improvement of atherosclerosis in neck arteries by ultrasonography from baseline to Week 16 and Week 32.

Additional secondary efficacy endpoints include the change from baseline in blood flow and atherosclerosis in neck arteries at Weeks 16 and 32. These variables will be measured at Day 1 (baseline), and Weeks 16 and 32.

### National Institute of Health Stroke Scale

The NIHSS is a serial measure of neurologic deficit and is used to objectively rate the severity of ischemic strokes. The scale is composed of 11 items summarizing a specific ability, such as level of consciousness, LOC questions, best gaze, visual, facial palsy, motor arm, motor leg, limb ataxia, sensory, best language, dysarthria, extinction and inattention (formerly neglect), with scores ranging from 0 to 4, with a smaller number indicating a more normal condition of the patient.

### Barthel Index

The BI is an ordinal scale used to measure a patient's performance in 10 individual activities of daily living (ADL). Each item is scored in increments of 5 points (0, 5, 10, or 15) and the individual items are summed to produce a total score between 0 and 100, where 0 is inferior performance and 100 is optimal. The lowest possible score 0 indicates total dependence on others for ADL, and the highest possible score 100 indicates full independence in ADL. A higher score is associated with a greater likelihood of being able to live at home with a degree of independence. A score of ≥95 is considered excellent.

### Evaluation: Safety

Safety assessments included AEs, vital signs (blood pressures, pulse rate, and oral temperature), clinical laboratories, physical examination, skin irritation, and ECGs at various time points during the study as indicated in the study flow chart.

Adverse events of interest included local skin reactions around the treated knee(s), upper stomach pain, GI bleeding, serious cardiovascular side effects (e.g., thrombotic events, myocardial infarction, or stroke), jaundice, elevated liver function tests, and nausea.

### Clinical Results

The clinical results (Primary and Secondary endpoints) are shown in Tables 11-14.

TABLE 11
Changes of Modified Rankin Scale Score (mRS) of 2-(diethylamino)ethyl
acetoxybenzoate hydrochloride vs Placebo

| | baseline | Week 4 | Week 8 | Week 16 | Week 24 | Week 32 |
|---|---|---|---|---|---|---|
| Placebo | 3.5 ± 0.5 | 3.3 ± 0.5 | 3.3 ± 0.5 | 3.3 ± 0.5 | 2.8 ± 0.4 | 2.7 ± 0.4 |
| 5 Sprays | 3.5 ± 0.5 | 3.1 ± 0.5 | 2.9 ± 0.3 | 2.7 ± 0.5 | 2.5 ± 0.5 | 2.4 ± 0.5 |
| 10 Sprays | 3.5 ± 0.5 | 2.5 ± 0.5 | 2.0 ± 0.5 | 1.6 ± 0.5 | 1.5 ± 0.5 | 1.4 ± 0.5 |
| 20 Sprays | 3.6 ± 0.5 | 2.1 ± 0.5 | 1.8 ± 0.4 | 1.4 ± 0.5 | 1.3 ± 0.5 | 1.3 ± 0.4 |

The results of mRS show that 2-(diethylamino)ethyl acetoxybenzoate hydrochloride can improve the functional recovery post strokes.

TABLE 12
Changes of National Institute of Health Stroke Scale of 2-(diethylamino)ethyl
acetoxybenzoate hydrochloride vs Placebo

| | baseline | Week 4 | Week 8 | Week 16 | Week 24 | Week 32 |
|---|---|---|---|---|---|---|
| Placebo | 5.8 ± 2.0 | 5.7 ± 1.9 | 5.7 ± 1.8 | 5.5 ± 1.6 | 4.0 ± 1.4 | 3.4 ± 0.9 |
| 5 Sprays | 6.0 ± 2.2 | 5.5 ± 1.7 | 5.3 ± 1.5 | 4.1 ± 1.0 | 3.4 ± 0.8 | 2.9 ± 0.6 |
| 10 Sprays | 5.9 ± 2.0 | 4.9 ± 1.5 | 3.7 ± 1.1 | 2.3 ± 0.8 | 1.6 ± 0.6 | 1.3 ± 0.4 |
| 20 Sprays | 6.1 ± 2.3 | 4.6 ± 1.6 | 3.0 ± 1.1 | 1.9 ± 0.9 | 1.4 ± 0.6 | 1.1 ± 0.3 |

The results of NIHSS show that 2-(diethylamino)ethyl acetoxybenzoate hydrochloride can improve the functional recovery post strokes.

TABLE 13
Changes of Barthel Index of 2-(diethylamino)ethyl
acetoxybenzoate hydrochloride vs Placebo

| | baseline | Week 4 | Week 8 | Week 16 | Week 24 | Week 32 |
|---|---|---|---|---|---|---|
| Placebo | 50.8 ± 13.4 | 51.4 ± 12.7 | 52.2 ± 12.4 | 53.2 ± 11.8 | 68.2 ± 9.7 | 70.6 ± 8.2 |
| 5 Sprays | 48.4 ± 11.0 | 52.3 ± 11.4 | 58.2 ± 11.3 | 65.2 ± 10.1 | 75.8 ± 8.8 | 83.6 ± 6.8 |
| 10 Sprays | 49.4 ± 11.8 | 58.9 ± 10.6 | 75.3 ± 9.9 | 92.2 ± 7.3 | 96.9 ± 3.9 | 99.0 ± 2.0 |
| 20 Sprays | 50.3 ± 12.5 | 61.5 ± 12.0 | 81.4 ± 9.7 | 95.5 ± 5.7 | 98.5 ± 2.9 | 99.4 ± 1.6 |

The results of BI show that 2-(diethylamino)ethyl acetoxybenzoate hydrochloride can improve the functional recovery post strokes.

TABLE 14
Improvement of Atherosclerosis in Neck Arteries by Ultrasonography
from Baseline to Week 16 and Week 32

| | | RCCA | RCCA | LCCA | LCCA | RICA | RICA | LICA | LICA |
|---|---|---|---|---|---|---|---|---|---|
| | | (near- | (far- | (near- | (far- | (near- | (far- | (near- | (far- |
| | | wall) | wall) | wall) | wall) | wall) | wall) | wall) | wall) |
| | | in mm | in mm | in mm | in mm | in mm | in mm | in mm | in mm |
| Base- | Placebo | 6.7 ± 1.5 | 7.7 ± 1.7 | 7.1 ± 1.6 | 5.6 ± 1.3 | 4.5 ± 1.2 | 3.7 ± 1.5 | 6.2 ± 1.7 | 4.6 ± 1.2 |
| line | 5 Sprays | 6.9 ± 2.1 | 7.1 ± 2.2 | 7.3 ± 2.0 | 6.2 ± 1.6 | 4.7 ± 1.5 | 4.1 ± 1.4 | 6.3 ± 2.1 | 5.3 ± 1.3 |
| | 10 Sprays | 7.2 ± 1.7 | 7.9 ± 2.1 | 7.0 ± 1.5 | 7.1 ± 1.7 | 5.1 ± 1.2 | 4.3 ± 1.5 | 6.1 ± 1.3 | 5.8 ± 1.5 |
| | 20 Sprays | 7.5 ± 1.8 | 8.8 ± 2.3 | 7.6 ± 1.6 | 6.5 ± 2.0 | 4.9 ± 1.3 | 4.7 ± 1.6 | 5.6 ± 1.7 | 5.7 ± 1.7 |
| Week | Placebo | 6.9 ± 1.8 | 7.5 ± 1.6 | 7.0 ± 1.8 | 5.8 ± 1.4 | 4.7 ± 1.5 | 4.2 ± 1.7 | 6.3 ± 1.9 | 5.1 ± 1.6 |
| 16 | 5 Sprays | 2.1 ± 0.9 | 2.5 ± 1.1 | 2.3 ± 1.3 | 2.7 ± 1.4 | 1.5 ± 1.2 | 1.6 ± 0.9 | 1.6 ± 1.3 | 1.5 ± 1.1 |
| | 10 Sprays | 1.6 ± 0.8 | 1.5 ± 0.6 | 1.3 ± 0.7 | 1.8 ± 0.7 | 0.9 ± 0.5 | 0.8 ± 0.3 | 0.9 ± 0.5 | 0.7 ± 0.3 |
| | 20 Sprays | 1.2 ± 0.5 | 1.4 ± 0.3 | 1.3 ± 0.5 | 1.2 ± 0.6 | 0.8 ± 0.3 | 0.6 ± 0.2 | 0.7 ± 0.3 | 0.6 ± 0.4 |
| Week | Placebo | 6.5 ± 1.5 | 7.3 ± 1.3 | 7.1 ± 1.7 | 5.6 ± 1.7 | 4.8 ± 1.4 | 4.0 ± 1.5 | 6.0 ± 1.7 | 4.9 ± 1.7 |
| 32 | 5 Sprays | 2.3 ± 0.8 | 2.6 ± 0.9 | 2.1 ± 0.7 | 2.5 ± 0.7 | 1.2 ± 0.6 | 1.1 ± 0.7 | 1.2 ± 1.0 | 1.3 ± 0.7 |
| | 10 Sprays | 1.3 ± 0.7 | 1.3 ± 0.8 | 1.2 ± 0.6 | 1.7 ± 0.8 | 0.7 ± 0.4 | 0.8 ± 0.2 | 0.7 ± 0.4 | 0.6 ± 0.3 |
| | 20 Sprays | 0.9 ± 0.4 | 1.0 ± 0.5 | 0.8 ± 0.5 | 1.1 ± 0.7 | 0.6 ± 0.5 | 0.5 ± 0.3 | 0.6 ± 0.3 | 0.5 ± 0.3 |

Note:
RCCA(near-wall): Maximal IMT of right common carotid artery(near-wall); RCCA(far-wall): Maximal IMT of right common carotid artery(far-wall); LCCA(near-wall): Maximal IMT of left common carotid artery(near-wall); LCCA(far-wall): Maximal IMT of left common carotid artery(far-wall); RICA(near-wall): Maximal IMT of right internal carotid artery(near-wall); RICA(far-wall): Maximal IMT of right internal carotid artery(far-wall); LICA(near-wall): Maximal IMT of left internal carotid artery(near-wall); LICA(far-wall): Maximal IMT of left internal carotid artery(far-wall).

The results of improvement of atherosclerosis show that 2-(diethylamino)ethyl acetoxybenzoate hydrochloride can reverse atherosclerosis.

### Safety Summary

All three doses of 2-(diethylamino)ethyl acetoxybenzoate appeared to be safe and were generally well tolerated. As stated previously, Gastrointestinal disorders are the major problem of all NSAIDs, but there were only 15 very mild incidences in this study (6 constipation, 3 diarrhea, 1 gastroesophageal reflux disorder, 2 abdominal discomfort, 1 abdominal pain, 1 abdominal pain upper and 1 nausea) and none of them appeared to be drug related. The incidence rates were generally similar across all three treatment groups. No notable upper GI tract ulcer complication (ie, bleeding episode, perforation, or gastric outlet obstruction) occurred during the study. Mean and median blood pressures remained unchanged. Even the skin irritation (a common AE of topical drugs) incidence rates were very low (total 6 incidences) and mild due to the simple formulation and fact that 2-(diethylamino)ethyl acetoxybenzoate is a biological inactive prodrug when it is on outside of the body.

Because 2-(diethylamino)ethyl acetoxybenzoate hydrochloride and other high penetration prodrugs of aspirin and other NSAIDs of the present disclosure are capable of crossing one or more biological barriers, they can be administered locally (e.g., topically or transdermally) to reach a location where a condition occurs and current drugs cannot reach significantly, such as the damaged brain tissues by strokes, the damaged heart tissues by heart attacks, the damaged heart tissues and blood vessels with heart failure, rheumatic heart disease, hypertensive heart disease, atrial fibrillation, congenital heart disease, endocarditis, aortic aneurysms, peripheral artery disease, atherosclerosis, and other cardiovascular diseases.

As shown by the reduction in the examples of animal model studies and human clinical trials, 2-(diethylamino)ethyl acetoxybenzoate hydrochloride can reduce the signs and symptoms of stroke, myocardial infarction, and/or cardiovascular diseases significantly in a dose response manner.

All 35 mg, 70 mg, and 140 mg BID of 2-(diethylamino)ethyl acetoxybenzoate hydrochloride appeared to be safe and were generally well tolerated. Gastrointestinal disorders are the major problem of all NSAIDs, but there were not drug-related gastrointestinal disorders in these studies. No notable upper GI tract ulcer complication (ie, bleeding episode, perforation, or gastric outlet obstruction) occurred during these studies. Mean and median blood pressures remained unchanged. Even the skin irritation (a common AE of topical drugs) incidence rates were very low and mild due to the simple formulation.

Although certain embodiments are described in detail above, they are only shown illustration and not as limitations of the invention. The principal features of this invention can be employed in various embodiments without departing from the scope of the invention. Those having ordinary skill in the art will clearly understand that many modifications are possible in the claims without departing from the teachings thereof. All such modifications and equivalents are intended to be encompassed within the claims of the invention and are covered by the claims. All publications, patents and other references mentioned herein are incorporated by reference in their entireties for all purposes.

1. A method of preventing or treating cardiovascular disease or condition in a subject, comprising topically administering a pharmaceutical composition comprising a compound of Formula (I), a stereoisomer thereof, or a pharmaceutically acceptable salt thereof to the subject: 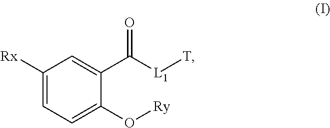
wherein:
Rx is selected from H, 2,4-difluorophenyl, substituted and unsubstituted alkyl, substituted and unsubstituted cycloalkyl, substituted and unsubstituted heterocyclyl, substituted and unsubstituted alkoxy, substituted and unsubstituted alkenyl, substituted and unsubstituted alkynyl, substituted and unsubstituted aryl, and substituted and unsubstituted heteroaryl;
Ry is selected from H, substituted and unsubstituted alkylcarbonyl, substituted and unsubstituted alkoxycarbonyl, substituted and unsubstituted benzoyl, substituted and unsubstituted alkyl, substituted and unsubstituted cycloalkyl, substituted and unsubstituted heterocyclyl, substituted and unsubstituted alkoxy, substituted and unsubstituted alkenyl, substituted and unsubstituted alkynyl, substituted and unsubstituted aryl, and substituted and unsubstituted heteroaryl;
L₁ is a linker selected from O, S, NH, O—CH(L2), O—(CH₂)n, O—CH(L₂)—O—C(═O), O—CH(L₂)-O, S—CH(L₂)-O, and —O—C(═O)—, wherein n is an integer selected from 1 to 6;
L₂ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino;
T is a transportational unit comprising a protonatable amine group.

2. The method according to claim 1, wherein the compound is selected from: 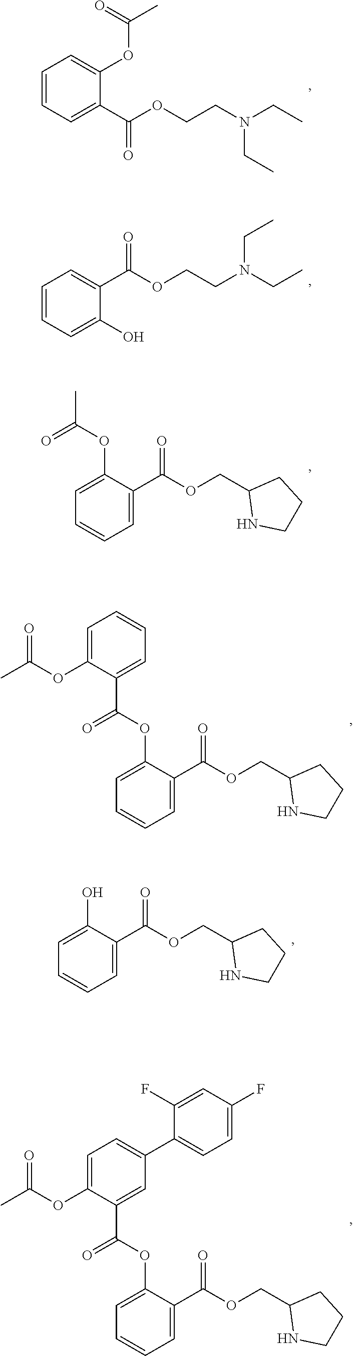 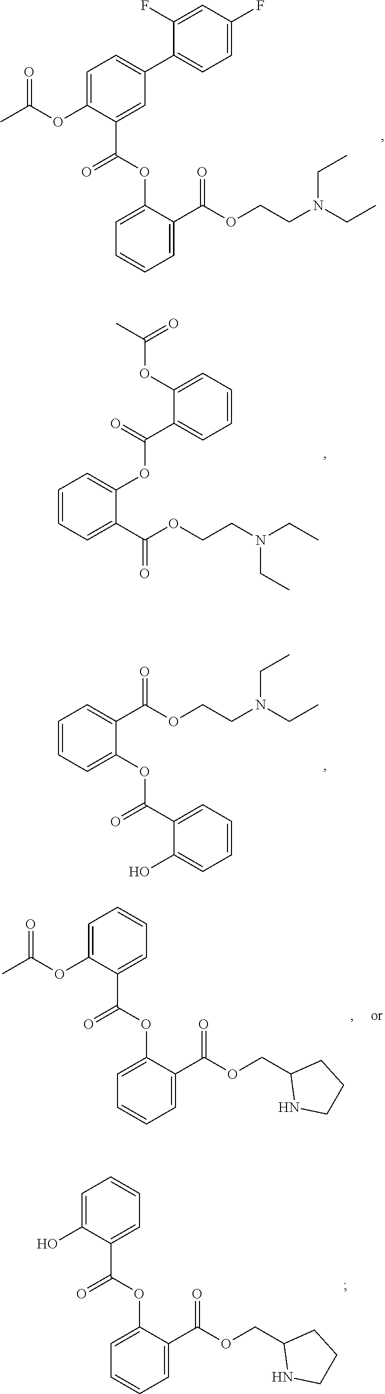
or a pharmaceutically acceptable salt thereof.

3. The method according to claim 1, wherein the pharmaceutical composition is in a dosage form selected from transdermal patch, cream, foam, gel, lotion, ointment, paste, powder, shake lotion, solid, sponge, tape, tincture, vapor, drops, rinces, spray, and solution.

4. The method according to claim 1, wherein the pharmaceutical composition is in a dosage form selected from an alcohol solution, an acetone solution, a dimethyl sulfoxide solution, an alcohol-water solution, an acetone-water solution, and a dimethyl sulfoxide-water solution.

5. The method according to claim 1, wherein the pharmaceutical composition is a solution having a concentration of 2-(diethylamino)ethyl acetoxybenzoate hydrochloride from about 10 mg/mL to about 200 mg/mL.

6. The method according to claim 1, wherein the pharmaceutical composition is a solution having a concentration of 2-(diethylamino)ethyl acetoxybenzoate hydrochloride from about 10 mg/g to about 200 mg/g.

7. The method according to claim 1, wherein the pharmaceutical composition is in a unit dose of about 0.01 mL to about 1 mL.

8. The method according to claim 1, wherein the pharmaceutical composition is topically administered to one or more sites of the subject in an amount of about 1 mg to about 7200 mg per day.

9. The method according to claim 8, wherein the pharmaceutical composition is topically administered to the subject in an amount from about 1 mg to about 700 mg per time once per day, twice per day, three times per day, or four times per day.

10. The method according to claim 8, wherein the pharmaceutical composition is topically administered to the subject in an amount from about 1 mg to about 200 mg per spray per site.

11. The method according to claim 8, wherein the pharmaceutical composition is topically administered to the subject in an amount from about 5 μg/cm² to about 7 mg/cm² skin per site.

12. The method according to claim 1, wherein said T is selected from Structure W-1, Structure W-2, Structure W-3, Structure W-4, Structure W-5, and Structure W-6; 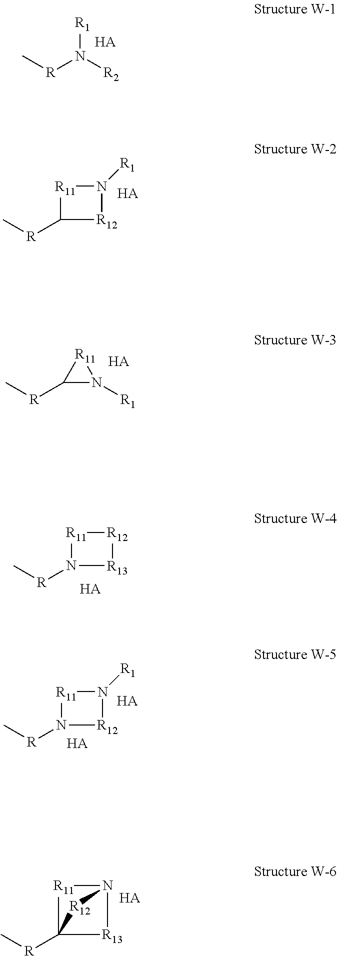
wherein,
R is independently selected from a bond, substituted and unsubstituted alkylene, substituted and unsubstituted cycloalkylene, substituted and unsubstituted heterocyclylene, substituted and unsubstituted alkenylene, substituted and unsubstituted alkynylene, substituted and unsubstituted arylene, and substituted and unsubstituted heteroarylene, wherein any CH₂ in R is optionally further replaced with O, S, or NR₃, wherein R₃ is hydrogen, C₁-C₆ alkyl, C₃-C₆ cycloalkyl, or C₆-C₁₀ aryl;
R₁ and R₂ are independently selected from H, substituted and unsubstituted alkyl, substituted and unsubstituted cycloalkyl, substituted and unsubstituted heterocyclyl, substituted and unsubstituted alkyloxy, substituted and unsubstituted alkenyl, substituted and unsubstituted alkynyl, substituted and unsubstituted aryl, and substituted and unsubstituted heteroaryl; or R₁ and R₂ together with the nitrogen atom to which they are attached form an optionally substituted heterocyclyl, which optionally further comprises one or two additional heteroatom(s) independently selected from O, S, and N;
R₁₁, R₁₂, and R₁₃ are each independently a bond, an optionally substituted C₁-C₄ alkylene, or an optionally substituted C₂-C₄ alkenylene, wherein the alkylene and alkenylene optionally has one CH₂ group replaced by O, S, or NR₃;
wherein any of the R₁ in Strucure W-2, Structure W-3 or Structure W-5 together and the adjacent R₁₁ together with the nitrogen atom to which they are attached may form an optionally substituted heterocyclic ring, which may optionally further comprise one or two additional heteroatom(s) independently selected from O, S, and N; and
wherein the R₁₁ and R₁₂ or R₁₁ and R₁₃ in Strucure W-2, Structure W-4, Structure W-5, or Structure W-6 are optionally connected by an alkylene bridge, which is optionally substituted; and
wherein HA is selected from nothing and pharmaceutically acceptable acids.

13. The method of claim 1, wherein the pharmaceutical composition is topically administered to sea skin surface of the subject at a site selected from neck, chest, back, abdomen, head, arms, hands, legs, feet, and combinations thereof.

14. The method of claim 1, wherein the pharmaceutical composition is topically administered to the subject in a single dose per site; and wherein the one or more unit doses are 1-200 unit doses.

15. The method of claim 1, wherein the pharmaceutical composition is topically administered to the subject once, twice, three times, four times, five times, six times, seven times or eight times a day.

16. The method of claim 1, wherein the pharmaceutical composition is topically administered to the subject once every 1, 2, 3, 4, 5, 6, 7, 8, 9, 10, 11, 12, 13, 14, 15, 16, 17, 18, 19, 20, 21, 22, 23, or 24 hours.

17. The method of claim 1, wherein the pharmaceutical composition is topically administered to the subject for a length from one day to life time.

18. The method of claim 1, wherein the cardiovascular disease or condition is selected from strokes, angina, myocardial infarction, heart failure, coronary artery diseases, rheumatic heart disease, hypertensive heart disease, atrial fibrillation, congenital heart disease, endocarditis, peripheral artery disease, atherosclerosis, and other cardiovascular diseases.

19. The method of claim 1, wherein the subject is a human.
